SEQUENCE LISTING (27 sequences)

SEQ ID NO: 1 - DNA, 2233 bp, Influenza virus
  source 1..2233: mol_type = other DNA
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagattttcacttcatcaatgagcaaggcgagtcaataatcgtagaacttggtgatccaaatgcacttttgaagcacagatttgaaataatcgagggaagagatcgcacaatggcctggacagtagtaaacagtatttgcaacactacaggggctgagaaaccaaagtttctaccagatttgtatgattacaaggagaatagattcatcgaaattggagtaacaaggagagaagttcacatatactatctggaaaaggccaataaaattaaatctgagaaaacacacatccacattttctcgttcactggggaagaaatggccacaaaggcagactacactctcgatgaagaaagcagggctaggatcaaaaccagactattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaagaaaacaagtcagctaaagtgggcacttggtgagaacatggcaccagaaaaggtagactttgacgactgtaaagatgtaggtgatttgaagcaatatgatagtgatgaaccagaattgaggtcgcttgcaagttggattcagaatgagtttaacaaggcatgcgaactgacagattcaagctggatagagctcgatgagattggagaagatgtggctccaattgaacacattgcaagcatgagaaggaattatttcacatcagaggtgtctcactgcagagccacagaatacataatgaagggagtgtacatcaatactgccttgcttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacataaatgggagaagtactgtgttcttgagataggagatatgcttataagaagtgccataggccaggtttcaaggcccatgttcttgtatgtgagaacaaatggaacctcaaaaattaaaatgaaatggggaatggagatgaggcgttgcctcctccagtcacttcaacaaattgagagtatgattgaagctgagtcctctgtcaaagagaaagacatgaccaaagagttctttgagaacaaatcagaaacatggcccattggagagtcccccaaaggagtggaggaaagttccattgggaaggtctgcaggactttattagcaaagtcggtattcaacagcttgtatgcatctccacaactagaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttccttacacatgcattgagttagttgtggcagtgctactatttgctatccatactgtccaaaaaagtaccttgtttctact

SEQ ID NO: 2 - DNA, 2340 bp, Influenza virus
  source 1..2340: mol_type = other DNA
agcgaaagcaggcaaaccatttgaatggatgtcaatccgaccttacttttcttaaaagtgccagcacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaggcgatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggatgtaatggagtcaatgaacaaagaagaaatggggatcacaactcattttcagagaaagagacgggtgagagacaatatgactaagaaaatgataacacagagaacaatgggtaaaaagaagcagagattgaacaaaaggagttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttcaccatcactggagataacaccaaatggaacgaaaatcagaatcctcggatgtttttggccatgatcacatatatgaccagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcatcgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacttggaatcaatatgagcaagaaaaagtcttacataaacagaacaggtacatttgaattcacaagttttttctatcgttatgggtttgttgccaatttcagcatggagcttcccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatataggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgatacaagtcaaagaggagtacttgaggatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 3 - DNA, 2341 bp, Influenza virus
  source 1..2341: mol_type = other DNA
agcgaaagcaggtcaattatattcaatatggaaagaataaaagaactacgaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgccggatcagaccgagtgatggtatcacctctggctgtgacatggtggaataggaatggaccaataacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaggaatgatgatgttgatcaaagcttgattattgctgctaggaacatagtgagaagagctgcagtatcagcagatccactagcatctttattggagatgtgccacagcacacagattggtggaattaggatggtagacatccttaggcagaacccaacagaagagcaagccgtggatatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatatgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagtcagaggtgatctgaatttcgtcaatagggcgaatcaacgattgaatcctatgcatcaacttttaagacattttcagaaggatgcgaaagtgctttttcaaaattggggagttgaacctatcgacaatgtgatgggaatgattgggatattgcccgacatgactccaagcatcgagatgtcaatgagaggagtgagaatcagcaaaatgggtgtagatgagtactccagcacggagagggtagtggtgagcattgaccgttttttgagaatccgggaccaacgaggaaatgtactactgtctcccgaggaggtcagtgaaacacagggaacagagaaactgacaataacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtaagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactataacaaggccacgaagagactcacagttctcggaaaggatgctggcactttaactgaagacccagatgaaggcacagctggagtggagtccgctgttctgaggggattcctcattctgggcaaagaagacaagagatatgggccagcactaagcatcaatgaactgagcaaccttgcgaaaggagagaaggctaatgtgctaattgggcaaggagacgtggtgttggtaatgaaacggaaacgggactctagcatacttactgacagccagacagcgaccaaaagaattcggatggccatcaattagtgtcgaatagtttaaaaacgaccttgtttctact

SEQ ID NO: 4 - DNA, 1565 bp, Influenza virus
  source 1..1565: mol_type = other DNA
agcaaaagcagggtagataatcactcactgagtgacatcaaaatcatggcgtctcaaggcaccaaacgatcttacgaacagatggagactgatggagaacgccagaatgccactgaaatcagagcatccgtcggaaaaatgattggtggaattggacgattctacatccaaatgtgcaccgaactcaaactcagtgattatgagggacggttgatccaaaacagcttaacaatagagagaatggtgctctctgcttttgacgaaaggagaaataaataccttgaagaacatcccagtgcggggaaagatcctaagaaaactggaggacctatatacaggagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagagctcttgttcgcaccggaatggatcccaggatgtgctctctgatgcaaggttcaactctccctaggaggtctggagccgcaggtgctgcagtcaaaggagttggaacaatggtgatggaattggtcagaatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagggagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattaagcttcatcaaagggacgaaggtgctcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccattatggcagcattcaatgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 5 - DNA, 1027 bp, Influenza virus
  source 1..1027: mol_type = other DNA
agcaaaagcaggtagatattgaaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcactattgccgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctact

SEQ ID NO: 6 - DNA, 890 bp, Influenza virus
  source 1..890: mol_type = other DNA
agcaaaagcagggtgacaaaaacataatggatccaaacactgtgtcaagctttcaggtagattgctttctttggcatgtccgcaaacgagttgcagaccaagaactaggcgatgccccattccttgatcggcttcgccgagatcagaaatccctaagaggaaggggcagtactctcggtctggacatcaagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcagaatggaccaggcgatcatggataagaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttagtactaaaaaacacccttgtttctact

SEQ ID NO: 7 - DNA, 1775 bp, Influenza virus
  source 1..1775: mol_type = other DNA
agcaaaagcaggggaaaataaaaacaaccaaaatgaaggcaaacctactggtcctgttatgtgcacttgcagctgcagatgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatctctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacattccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctgagattagaatttcagagatatgaggaaaaacacccttgtttctact

SEQ ID NO: 8 - DNA, 1413 bp, Influenza virus
  source 1..1413: mol_type = other DNA
agcaaaagcaggggtttaaaatgaatccaaatcagaaaataataaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgacccaaggtgccttactgaatgacaagcattcaagtgggactgttaaggacagaagcccttatagggccttaatgagctgccctgtcggtgaagctccgtccccgtacaattcaagatttgaatcggttgcttggtcagcaagtgcatgtcatgatggcatgggctggctaacaatcggaatttcaggtccagataatggagcagtggctgtattaaaatacaacggcataataactgaaaccataaaaagttggaggaagaaaatattgaggacacaagagtctgaatgtgcctgtgtaaatggttcatgttttactataatgactgatggcccgagtgatgggctggcctcgtacaaaattttcaagatcgaaaaggggaaggttactaaatcaatagagttgaatgcacctaattctcactatgaggaatgttcctgttaccctgataccggcaaagtgatgtgtgtgtgcagagacaattggcatggttcgaaccggccatgggtgtctttcgatcaaaacctggattatcaaataggatacatctgcagtggggttttcggtgacaacccgcgtcccgaagatggaacaggcagctgtggtccagtgtatgttgatggagcaaacggagtaaagggattttcatataggtatggtaatggtgtttggataggaaggaccaaaagtcacagttccagacatgggtttgagatgatttgggatcctaatggatggacagagactgatagtaagttctctgtgaggcaagatgttgtggcaatgactgattggtcagggtatagcggaagtttcgttcaacatcctgagctgacagggctagactgtatgaggccgtgcttctgggttgaattaatcaggggacgacctaaagaaaaaacaatctggactagtgcgagcagcatttctttttgtggcgtgaatagtgatactgtagattggtcttggccagacggtgctgagttgccattcagcattgacaagtagtctgttcaaaaaactccttgtttctact

SEQ ID NO: 9 - skipped

SEQ ID NO: 10 - DNA, 2342 bp, Influenza virus
  source 1..2342: mol_type = other DNA
agcgaaagcaggcaaaccatttgaatggatgtcaatccgaccttacttttcttaaaagtgccagcacaaaatgctataagcacaactttcccttataccggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacactgagtagacaagctgacacaaggccgacagacctatgactggactttaaatagaaaccagcctgctgcaacagcattggccaacacaatagaagtgttcagatcaaatggcctcacggccaatgagtcaggaaggctcatagacttccttaaggatgtaatggagtcaatgaaaaaagaagaaatggggatcacaactcattttcagagaaagagacgggtgagagacaatatgactaagaaaatgataacacagagaacaataggtaaaaggaaacagagattgaacaaaaggggttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttcaccatcactggagataacaccaaatggaacgaaaatcagaatcctcggatgtttttggccatgatcacatatatgaccagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcattgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacttggaatcaatatgagcaagaaaaagtcttacataaacagaacaggtacatttgaattcacaagttttttctatcgttatgggtttgttgccaatttcagcatggagcttcccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatagaggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaagatgaacaaatgtaccaaaggtgctggaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 11 - DNA, 2341 bp, Influenza virus
  source 1..2341: mol_type = other DNA
agcgaaagcaggtcaattatattcaatatggaaagaataaaagaactaagaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgccggatcagaccgagtgatggtatcacctctggctgtgacatggtggaataggaatggaccaatgacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaagaatgatgatgttgatcaaagcttgattattgctgctaggaacatagtgagaagagctgcagtatcagcagacccactagcatctttattggagatgtgccacagcacacagattggtggaattaggatggtagacatccttaagcagaacccaacagaagagcaagccgtggatatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatctgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagttagaggtgatctgaatttcgtcaatagggcgaatcagcgactgaatcctatgcatcaacttttaagacattttcagaaggatgcgaaagtgctttttcaaaattggggagttgaacctatcgacaatgtgatgggaatgattgggatattgcccgacatgactccaagcatcgagatgtcaatgagaggagtgagaatcagcaaaatgggtgtagatgagtactccagcacggagagggtagtggtgagcattgaccggttcttgagagtcagggaccaacgaggaaatgtactactgtctcccgaggaggtcagtgaaacacagggaacagagaaactgacaataacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtaagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactacaacaaggccacgaagagactcacagttctcggaaaggatgctggcactttaaccgaagacccagatgaaggcacagctggagtggagtccgctgttctgaggggattcctcattctgggcaaagaagacaggagatatgggccagcattaagcatcaatgaactgagcaaccttgcgaaaggagagaaggctaatgtgctaattgggcaaggagacgtggtgttggtaatgaaacgaaaacgggactctagcatacttactgacagccagacagcgaccaaaagaattcggatggccatcaattagtgtcgaatagtttaaaaacgaccttgtttctact

SEQ ID NO: 12 - DNA, 2234 bp, Influenza virus
  source 1..2234: mol_type = other DNA
agcgaaagcaggtactgattcaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagatttccacttcatcaatgagcaaggcgagtcaataatcgtagaacttggtgatcctaatgcacttttgaagcacagatttgaaataatcgagggaagagatcgcacaatggcctggacagtagtaaacagtatttgcaacactacaggggctgagaaaccaaagtttctaccagatttgtatgattacaaggaaaatagattcatcgaaattggagtaacaaggagagaagttcacatatactatctggaaaaggccaataaaattaaatctgagaaaacacacatccacattttctcgttcactggggaagaaatggccacaagggccgactacactctcgatgaagaaagcagggctaggatcaaaaccaggctattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaaaaaaacaagtcagctaaagtgggcacttggtgagaacatggcaccagaaaaggtagactttgacgactgtaaagatgtaggtgatttgaagcaatatgatagtgatgaaccagaattgaggtcgcttgcaagttggattcagaatgagttcaacaaggcatgcgaactgacagattcaagctggatagagcttgatgagattggagaagatgtggctccaattgaacacattgcaagcatgagaaggaattatttcacatcagaggtgtctcactgcagagccacagaatacataatgaagggggtgtacatcaatactgccttacttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacacaaatgggagaagtactgtgttcttgagataggagatatgcttctaagaagtgccataggccaggtttcaaggcccatgttcttgtatgtgaggacaaatggaacctcaaaaattaaaatgaaatggggaatggagatgaggcgttgtctcctccagtcacttcaacaaattgagagtatgattgaagctgagtcctctgtcaaagagaaagacatgaccaaagagttctttgagaacaaatcagaaacatggcccattggagagtctcccaaaggagtggaggaaagttccattggggaaggtctgcaggactttattagcaaagtcggtatttaacagcttgtatgcatctccacaactagaaggattttcagctgaatcaagaaaactgcttcttatcgttcaggctcttagggacaatctggaacctgggacctttgatcttggggggctatatgaagcaattgaggagtgcctaattaatgatccctgggttttgcttaatgcttcttggttcaactccttccttacacatgcattgagttagttgtggcagtgctactatttgctatccatactgtccaaaaaagtaccttgtttctact

SEQ ID NO: 13 - DNA, 1565 bp, Influenza virus
  source 1..1565: mol_type = other DNA
agcaaaagcagggtagataatcactcactgagtgacatcaaaatcatggcgtcccaaggcaccaaacggtcttacgaacagatggagactgatggagaacgccagaatgccactgaaatcagagcatccgtcggaaaaatgattggtggaattggacgattctacatccaaatgtgcacagaacttaaactcagtgattatgagggacggttgatccaaaacagcttaacaatagagagaatggtgctctctgcttttgacgaaaggagaaataaatacctggaagaacatcccagtgcggggaaagatcctaagaaaactggaggacctatatacagaagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagggctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattgagcttcatcaaagggacgaaggtggtcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccgttatggcagcattcactgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 14 - DNA, 1027 bp, Influenza virus
  source 1..1027: mol_type = other DNA
agcaaaagcaggtagatattgaaagatgagtcttctaaccgaggtcgaaacgtacgttctctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaacccactaatcagacatgagaacagaatggttttagccagcactacagctaaggctatggagcaaatggctggatcgagtgagcaagcagcagaggccatggaggttgctagtcaggctaggcaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcgctattgccgcaaatatcattgggatcttgcacttgatattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctact

SEQ ID NO: 15 - DNA, 890 bp, Influenza virus
  source 1..890: mol_type = other DNA
agcaaaagcagggtgacaaagacataatggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcactcttggtctggacatcgagacagccacacgtgctggaaagcagatagtggagcggattctgaaagaagaatccgatgaggcacttaaaatgaccatggcctctgtacctgcgtcgcgttacctaaccgacatgactcttgaggaaatgtcaagggaatggtccatgctcatacccaagcagaaagtggcaggccctctttgtatcagaatggaccaggcgatcatggataaaaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaaggtaacagagaatagttttgagcaaataacatttatgcaagccttacatctattgcttgaagtggagcaagagataagaactttctcatttcagcttatttaataataaaaaacacccttgtttctact

SEQ ID NO: 16 - DNA, 2341 bp, Influenza virus
  source 1..2341: mol_type = other DNA
agcgaaagcaggtcaattatattcaatatggaaagaataaaagaactacgaaatctaatgtcgcagtctcgcacccgcgagatactcacaaaaaccaccgtggaccatatggccataatcaagaagtacacatcaggaagacaggagaagaacccagcactgaggatgaaatggatgatggcaatgaaatatccaattacagcagacaagaggatcaccgaaatgattcctgagagaaatgagcagggacagactctgtggagtaaaatgaatgatgccggatcagaccgagtgatggtgtcacctctggctgtgacatggtggaataggaatggaccaatcacaaatacagtgcattatccaaaaatctacaaaacttattttgaaagagtcgaaaggctgaagcatggaacctttggccctgtccattttagaaaccaggtcaaaatccggcggagagtggacatcaatcctggtcatgcagatctcagtgccaaggaggcacaggatgtgatcatggaagtggtgttccctaacgaagtgggagccaggattctgacatccgaatcccagctgaccattaccaaagagaagaaagaagaactccaggattgcaaaatttctcctctgatggtggcatacatgctggagagagaactggtccgcaaaacaagattcctcccagtggctggtggaacaagcagtgtgtacattgaagtgctgcatctgactcagggaacatgctgggaacagatgtatactccaggaggggaagtgaggaatgatgatgtggatcagagcctgattattgctgctaggaacattgtgagaagagctgcagtgtcagcagatccactggcatctctgctggagatgtgccacagcacacagattggtggaattaggatggtggacatcctgaggcagaacccaacagaagagcaggccgtggatatttgcaaggctgcaatgggactgagaattagctcatccttcagttttggtggattcacatttaagagaacaagcggatcatcagtcaagagagaggaagaggtgctgaccggcaatctgcagacactgaagatcagagtgcatgagggatatgaagagttcacaatggtggggagaagagcaacagccatcctcagaaaagcaaccaggagactgattcagctgatcgtgagtgggagagacgaacagtccattgccgaagcaattattgtggccatggtgttttcacaggaggattgtatgattaaagcagtcagaggtgatctgaatttcgtcaatagggccaatcagcgactgaatcctatgcatcagctgctgagacattttcagaaggatgccaaagtgctgtttcagaattggggagtggaacctatcgacaatgtgatgggaatgattgggatcctgcccgacatgactccaagcatcgagatgtcaatgagaggagtgagaatcagcaaaatgggtgtggatgagtactccagcaccgagagggtcgtggtgagcattgacagatttctgagaatccgggaccagcgaggaaatgtgctcctgtctcccgaggaggtcagtgaaacacagggaacagagaaactgacaattacttactcatcctcaatgatgtgggagattaatggtcctgaatcagtgctggtcaatacctatcagtggatcatcagaaactgggaaactgtgaaaattcagtggtcccagaaccctacaatgctgtacaataaaatggaatttgaaccatttcagtctctggtgcctaaggccattagaggccagtacagtgggtttgtgagaactctgttccagcagatgagggatgtgctggggacatttgataccgcacagattattaaactgctgcccttcgcagccgctccaccaaagcagagtagaatgcagttctcctcatttactgtgaatgtgaggggatcaggaatgagaatcctggtgaggggcaattctcctgtgttcaactataacaaggccaccaagagactcacagtgctcggaaaggatgctggcactctgactgaagacccagatgaaggcacagctggagtggagtccgctgtgctgaggggattcctcattctgggcaaagaagacaagagatatgggccagcactgagcatcaatgaactgagcaacctggccaaaggagagaaggctaatgtgctaattgggcaaggagacgtggtgttggtaatgaaacggaaacgggactctagcatacttactgacagccagacagcgaccaaaagaattcggatggccatcaattagtgtcgaatagtttaaaaacgaccttgtttctact

SEQ ID NO: 17 - DNA, 2341 bp, Influenza virus
  source 1..2341: mol_type = other DNA
agcgaaagcaggcaaaccatttgaatggatgtcaatccgaccttacttttcttaaaagtgccagcacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaggcgatggctttccttgaggaatcccatcctggtatttttgaaaactcgtgtattgaaacgatggaggttgttcagcaaacacgagtggacaagctgacacagggccgacagacctatgactggactctgaatagaaaccagcctgctgcaacagcactggccaacacaatcgaagtgttcagatcaaatggcctcaccgccaatgagtctggaaggctcatcgacttcctgaaggatgtgatggagtcaatgaacaaagaagaaatggggatcacaactcattttcagagaaagagacgggtgagagacaatatgactaagaaaatgattacacagagaacaatgggtaaaaagaagcagagactgaacaaaaggagttatctgattagagcactgaccctgaacacaatgaccaaagatgctgagagagggaagctgaaacggagagcaattgcaaccccagggatgcagattagggggtttgtgtactttgtggagacactggcaaggagtatttgtgagaaactggaacagtcagggctgccagtgggaggcaatgagaagaaagcaaagctggcaaatgtggtgaggaagatgatgaccaattctcaggacaccgaactgtctttcaccatcactggagataacaccaaatggaacgaaaatcagaatcctcggatgtttctggccatgatcacatatatgaccagaaatcagcccgaatggttcagaaatgtgctgagtattgctccaattatgttctcaaacaaaatggccagactgggaaaagggtatatgtttgagagcaagagtatgaaactgagaactcagattcctgcagaaatgctggcaagcatcgatctgaaatatttcaatgattcaacaagaaagaagattgaaaaaatccgacccctcctgattgaggggactgcatcactgagccctggaatgatgatgggcatgttcaatatgctgagcactgtgctgggcgtctccatcctgaatctgggacagaagagatacaccaagactacttactggtgggatggtctgcagtcctctgacgattttgctctgattgtgaatgcacccaatcatgaagggattcaggccggagtcgacaggttttatcgaacctgtaagctgctgggaatcaatatgagcaagaaaaagtcttacatcaacagaacaggtacatttgaattcacaagttttttctatcgctatgggtttgtggccaatttcagcatggagctgcccagttttggggtgtctgggatcaacgagtcagccgacatgagtattggagtgactgtcatcaaaaacaatatgatcaacaatgatctgggtccagcaacagctcagatggccctgcagctgttcatcaaagattacaggtacacctaccgatgccatatcggtgacacacagattcagacccgaagatcatttgaaatcaagaaactgtgggagcagacccgctccaaagctggactgctggtctccgacggaggcccaaatctgtacaacattagaaatctccacattcctgaagtctgcctgaaatgggaactgatggatgaggattaccaggggcgcctgtgcaacccactgaacccatttgtcagccataaagaaattgaatcaatgaacaatgcagtgatgatgccagcacatggtccagccaaaaacatggagtatgatgctgtggcaacaacacactcctggatccccaaaagaaatcgatccatcctgaatacaagtcagagaggagtgctggaggatgaacagatgtaccagaggtgctgcaatctgtttgaaaaattcttccccagcagttcatacagaagaccagtcgggatctccagtatggtggaggctatggtgtccagagcccgaattgatgcacggattgatttcgaatctggaaggatcaagaaagaagagttcactgagatcatgaagatctgttccaccattgaagagctcagacggcaaaaatagtgaatttagcttgtccttcatgaaaaaatgccttgtttctact

SEQ ID NO: 18 - DNA, 2233 bp, Influenza virus
  source 1..2233: mol_type = other DNA
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagattttcacttcatcaatgagcaaggcgagtcaataatcgtagaacttggtgatccaaatgcacttttgaagcacagatttgaaataatcgagggaagagatcgcacaatggcctggacagtagtaaacagtatttgcaacactacaggggctgagaaaccaaagtttctaccagatttgtatgattacaaggagaatagattcatcgaaattggagtaacaaggagagaagttcacatatactatctggaaaaggccaataaaattaaatctgagaaaacacacatccacattttctcgttcactggggaagaaatggccacaaaggcagactacactctcgatgaagaaagcagggctaggatcaaaaccagactattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttctgaaaacaacaccacgaccactgagactgcccaatgggcctccctgttctcagcggtccaaattcctgctgatggatgccctgaaactgagcattgaggacccaagtcatgaaggagagggaattcccctgtatgatgcaatcaaatgcatgagaacattctttggatggaaggaacccaatgtggtgaaaccacacgaaaagggaatcaatccaaattatctgctgtcatggaagcaggtgctggcagaactgcaggacattgagaatgaggagaaaattccaaagactaaaaatatgaagaaaacaagtcagctgaagtgggcactgggtgagaacatggcaccagaaaaggtggactttgacgactgtaaagatgtgggtgatctgaagcagtatgatagtgatgaaccagaactgaggtccctggcaagttggattcagaatgagtttaacaaggcatgcgaactgacagattcaagctggattgagctcgatgagattggagaagatgtggctccaattgaacacattgcaagcatgagaaggaattatttcacatcagaggtgtctcactgcagagccacagaatacatcatgaagggagtgtacatcaatactgccctgctgaatgcatcttgtgcagcaatggatgatttccagctgattccaatgatcagcaagtgtagaactaaggagggaaggcgaaagaccaacctgtatggtttcatcatcaaaggaagatcccacctgaggaatgacaccgacgtggtgaactttgtgagcatggagttttctctcactgacccaagactggaaccacataaatgggagaagtactgtgtgctggagattggagatatgctgatcagaagtgccattggccaggtgtcaaggcccatgttcctgtatgtgagaacaaatggaacctcaaaaattaaaatgaaatggggaatggagatgaggcgctgcctcctccagtcactgcagcagattgagagtatgattgaagctgagtcctctgtcaaagagaaagacatgaccaaagagttctttgagaacaaatcagaaacatggcccattggagagtcccccaaaggagtggaggaaagttccattgggaaggtctgcaggactctgctggcaaagtccgtgttcaacagcctgtatgcatctccacagctggaaggattttcagctgaatcaagaaaactgctgctgatcgtgcaggctctgagggacaacctggaacctgggacctttgatctgggggggctgtatgaagcaattgaggagtgcctgattaatgatccctgggtgctgctgaatgcttcttggttcaactccttccttacacatgcattgagttagttgtggcagtgctactatttgctatccatactgtccaaaaaagtaccttgtttctact

SEQ ID NO: 19 - DNA, 1565 bp, Influenza virus
  source 1..1565: mol_type = other DNA
agcaaaagcagggtagataatcactcactgagtgacatcaaaatcatggcgtctcaaggcaccaaacgatcttacgaacagatggagactgatggagaacgccagaatgccactgaaatcagagcatccgtcggaaaaatgattggtggaattggacgattctacatccagatgtgcaccgaactcaaactcagtgattatgagggacggctgatccagaacagcctgacaatcgagagaatggtgctctctgcttttgacgaaaggagaaataaatacctggaagaacatcccagtgccgggaaagatcctaagaaaactggaggacctatctacaggagagtgaacggaaagtggatgagagaactcatcctgtatgacaaagaagaaatcaggcgaatctggcgccaggctaataatggtgacgatgcaaccgctggtctgactcacatgatgatctggcattccaatctgaatgatgcaacttatcagaggacaagagctctggtgcgcaccggaatggatcccaggatgtgctctctgatgcagggttcaactctccctaggaggtctggagccgcaggtgctgcagtcaaaggagtgggaacaatggtgatggaactggtcagaatgatcaaaagagggatcaatgatcggaacttctggaggggtgagaatggacgaaaaacaagaattgcttatgaaagaatgtgcaacattctcaaagggaaatttcagactgctgcacagaaagcaatgatggatcaggtgagagagagccggaacccagggaatgctgagttcgaagatctcacttttctggcacggtctgcactcatcctgagagggtccgtggctcacaagtcctgcctgcctgcctgtgtgtatggacctgccgtggccagtgggtacgactttgaaagggagggatactctctggtcggaattgaccctttcagactgctgcagaacagccaggtgtacagcctgatcagaccaaatgagaatccagcacacaagagtcagctggtgtggatggcatgccattctgccgcatttgaagatctgagagtgctgagcttcatcaaagggaccaaggtgctcccaagagggaagctgtccactagaggagtgcagattgcttccaatgaaaatatggagactatggaatcaagtacactggaactgagaagcaggtactgggccatcaggaccagaagtggaggaaacaccaatcagcagagggcatctgccggccagatcagcattcagcctaccttctcagtgcagagaaatctcccttttgacagaacaaccattatggcagcattcaatgggaatacagaggggagaacatctgacatgaggaccgaaatcatcaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 20 - DNA, 1684 bp, Influenza virus
  source 1..1684: mol_type = other DNA
atgaacactcaaatcctggtattcgctctgattgcgatcattccaacaaatgcagacaaaatctgcctcggacatcatgccgtgtcaaacggaaccaaagtaaacacattaactgaaagaggagtggaagtcgtcaatgcaactgaaacagtggaacgaacaaacatccccaggatctgctcaaaagggaaaaggacagttgacctcggtcaatgtggactcctggggacaatcactggaccacctcaatgtgaccaattcctagaattttcagccgatttaattattgagaggcgagaaggaagtgatgtctgttatcctgggaaattcgtgaatgaagaagctctgaggcaaattctcagagaatcaggcggaattgacaaggaagcaatgggattcacatacagtggaataagaactaatggagcaaccagtgcatgtaggagatcaggatcttcattctatgcagaaatgaaatggctcctgtcaaacacagataatgctgcattcccgccagatgactaagtcatataaaaatacaagaaaaagcccagctctaatagtatgggggatccatcattccgtatcaactgcagagcaaaccaagctatatgggagtggaaacaaactggtgacagttgggagttctaattatcaacaatcttttgtaccgagtccaggagcgagaccacaagttaatggtctatctggaagaattgactttcattggctaatgctaaatcccaatgatacagtcactttcagtttcaatggggctttcatagctccagaccgtgcaagcttcctgagaggaaaatctatgggaatccagagtggagtacaggttgatgccaattgtgaaggggactgctatcatagtggagggacaataataagtaacttgccatttcagaacatagatagcagggcagttggaaaatgtccgagatatgttaagcaaaggagtctgctgctagcaacagggatgaagaatgttcctgagattccaaagggaagaggcctatttggtgctatagcgggtttcattgaaaatggatgggaaggcctaattgatggttggtatggtttcagacaccagaatgcacagggagagggaactgctgcagattacaaaagcactcaatcggcaattgatcaaataacaggaaaattaaaccggcttatagaaaaaaccaaccaacaatttgagttgatagacaatgaattcaatgaggtagagaagcaaatcggtaatgtgataaattggaccagagattctataacagaagtgtggtcatacaatgctgaactcttggtagcaatggagaaccagcatacaattgatctggctgattcagaaatggacaaactgtacgaacgagtgaaaagacagctgagagagaatgctgaagaagatggcactggttgctttgaaatatttcacaagtgtgatgatgactgtatggccagtattagaaataacacctatgatcacagcaaatacagggaagaggcaatgcaaaatagaatacagattgacccagtcaaactaagcagcggctacaaagatgtgatactttggtttagcttcggggcatcatgtttcatacttctagccattgtaatgggccttgtcttcatatgtgtaaagaatggaaacatgcggtgcactatttgtatataa

SEQ ID NO: 21 - protein, 560 aa, Influenza virus
MNTQILVFALSKGKRTVDLGIAIIPTNADKQCGLLGTITGICLGHHAVSNPPQCDQFLEFGTKVNTLTERSADLIIERREGVEVVNATETGSDVCYPGKFVERTNIPRICVNEEALRQILRESGGIDKEAMGFTYSGIRTNGATSACRRSGSSFYAEMKWLLSNTDNAAFPQMTKSYKNTRKSPALIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDANCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 22 - DNA, 1398 bp, Influenza virus
  source 1..1398: mol_type = other DNA
atgaatccaaatcagaagattctatgcacttcagccactgctatcataataggcgcaatcgcagtactcattggaatagcaaacctaggattgaacataggactgcatctaaaaccgggctgcaattgctcacactcacaacctgaaacaaccaacacaagccaaacaataataaacaactattataatgaaacaaacatcaccaacatccaaatggaagagagaacaagcaggaatttcaataacttaactaaagggctctgtactataaattcatggcacatatatgggaaagacaatgcagtaagaattggagagagctcggatgttttagtcacaagagaaccctatgtttcatgcgacccagatgaatgcaggttctatgctctcagccaaggaacaacaatcagagggaaacactcaaacggaacaatacacgataggtcccagtatcgcgccctgataagctggccactatcatcaccgcccacagtgtacaacagcagggtggaatgcattgggtggtcaagtactagttgccatgatggcaaatccaggatgtcaatatgtatatcaggaccaaacaacaatgcatctgcagtagtatggtacaacagaaggcctgttgcagaaattaacacatgggcccgaaacatactaagaacacaggaatctgaatgtgtatgccacaacggcgtatgcccagtagtgttcaccgatgggtctgccactggacctgcagacacaagaatatactattttaaagaggggaaaatattgaaatgggagtctctgactggaactgctaagcatattgaagaatgctcatgttacggggaacgaacaggaattacctgcacatgcagggacaattggcagggctcaaatagaccagtgattcagatagacccagtagcaatgacacacactagtcaatatatatgcagtcctgttcttacagacaatccccgaccgaatgacccaaatataggtaagtgtaatgacccttatccaggtaataataacaatggagtcaagggattctcatacctggatggggctaacacttggctagggaggacaataagcacagcctcgaggtctggatacgagatgttaaaagtgccaaatgcattgacagatgatagatcaaagcccattcaaggtcagacaattgtattaaacgctgactggagtggttacagtggatctttcatggactattgggctgaaggggactgctatcgagcgtgtttttatgtggagttgatacgtggaagacccaaggaagataaagtgtggtggaccagcaatagtatagtatcgatgtgttccagtacagaattcctgggacaatggaactggcctgatggggctaaaatagagtacttcctctaa

SEQ ID NO: 23 - protein, 464 aa, Influenza virus
MNPNQKILCTSATAIIIGAIAVLIGIANLGLNIGLHLKPGCNCSHSQPETTNTSQTIINNYYNETNITNIQMEERTSRNFNNLTKGLCTINSWHIYGKDNAVRIGESSDVLVTREPYVSCDPDECRFYALSQGTIIRGKHSNGTIHDRSQYRALISWPLSSPPTVYNSRVECIGWSSTSCHDGKSRMSICISGPNNNASAVVWYNRRPVAEINTWARNILRTQESECVCHNGVCPVVFTDGSATGPADTRIYFKEGKILKWESLTGTAKHIEECSCYGERTGITCTCRDNWQGSNRPVIQIDPVAMTHTSQYICSPVLTDNPRPNDPNIGKCNDPYPGNNNNGVKGFSYLDGANTWLGRTISTASRSGYEMLKVPNALTDDRSKPIQGQTIVLNADWSGYSGSFMDYWAEGDCYRACFYVELIRGRPKEDKVWWTSNSIVSMCSSTEFLGQWNWPDGAKIEYFL

SEQ ID NO: 24 - DNA, 1683 bp, Influenza virus
  source 1..1683: mol_type = other DNA
atgaacactcaaatcctggtattcgctctgattgcgatcattccaacaaatgcagacaaaatctgcctcggacatcatgctgtgtcaaacggaaccaaagtaaacacattaactgaaagaggagtggaagtcgtcaatgcaactgaaacagtggaacgaacaaacatccccaggatctgctcaaaagggaaaaggacagttgacctcggtcaatgtggactcctggggacaatcactggaccacctcaatgtgaccaattcctagaattttcagccgatttaattattgagaggcgagaaggaagtgatgtctgttatcctgggaaattcgtgaatgaagaagctctgaggcaaattctcagagaatcaggcggaattgacaaggaagcaatgggattcacatacagtggaataagaactaatggagcaaccagttcatgtaggagatcaggatcttcattctatgcagaaatgaaatggctcctgtcaaacacagataatgctgcattcccgcagatgactaagtcatataaaaatacaagaaaaaacccagctctaatagtatgggggatccatcattccggatcaactgcagagcaaaccaagctatatgggagtggaaacaaactggtgacagttgggagttctaattatcaacaatcttttgtaccgagtccgggagcgagaacacaagttaatggtcaatctggaagaattgactttcattggctaatgctaaatcccaatgatacagtcactttcagtttcaatggggctttcatagctccagaccgtgcaagcttcctgagaggaaaatctatgggaatccagagtggagtacaggttgatgccgattgtgaaggggactgctattatagtggagggacaataataagtaacttgccatttcagaacatagatagcagggcagttggaaaatgtccgagatatgttaagcaaaggagtctgctgctagcaacagggatgaagaatgttcctgagattccaaagggaagaggcctatttggtgctatagcgggtttcattgaaaatggatgggaaggcctaattgatggttggtatggtttcagacaccagaatgcacagggagagggaactgctgcagattacaaaagcactcaatcggcaattgatcaaataacaggaaaattaaaccggcttatagaaaaaaccaaccaacaatttgagttgatagacaatgaattcactgaggtagagaagcaaatcggtaatgtgataaattggaccagagattctataacagaagtgtggtcatacaatgctgaactcttggtagcaatggagaaccagcatacaattgatctggctgattcagaaatggacaaactgtacgaacgagtgaaaagacagctgagagagaatgctgaagaagatggcactggttgctttgaaatatttcacaagtgtgatgatgactgtatggccagcattagaaataacacctatgatcacagcaaatacagggaagaggcaatgcaaaatagaatacagattgacccagtcaaactaagcagcggctacaaagatgtgatactttggtttagcttcggggcatcatgtttcatacttctagccattgcaatgggccttgtcttcatatgtgtaaagaatggaaacatgcggtgcactatttgtatataa

SEQ ID NO: 25 - protein, 560 aa, Influenza virus
MNTQILVFALIAIIPTNADKICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRTNGATSSCRRSGSSFYAEMKWLLSNTDNAAFPQMTKSYKNTRKNPALIVWGIHHSGSTAEQTKLYGSGNKLVTVGSSNYQQSFVPSPGARTQVNGQSGRIDFHWLMLNPNDTVTFSFNGAFIAPDRASFLRGKSMGIQSGVQVDADCEGDCYYSGGTIISNLPFQNIDSRAVGKCPRYVKQRSLLLATGMKNVPEIPKGRGLFGAIAGFIENGWEGLIDGWYGFRHQNAQGEGTAADVKSTQSAIDQITGKLNRLIEKTNQQFELIDNEFTEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIAMGLVFICVKNGNMRCTICI

SEQ ID NO: 26 - DNA, 1398 bp, Influenza virus
  source 1..1398: mol_type = other DNA
atgaatccaaatcagaagattctatgcacttcagccactgctatcataataggcgcaatcgcagtactcattggaatagcaaacctaggattgaacataggactgcatctaaaaccgagctgcaattgctcacactcacaacctgaaacaaccaacacaagccaaacaataataaacaactattataatgaaacaaacatcaccaacatccaaatggaagagagaacaagcaggaatttcaataacttaactaaagggctctgtactataaattcatggcacatatatgggaaagacaatgcggtaagaattggagagagctcggatgttttagtcacaagagaaccctatgtttcatgcgacccagatgaatgcaggttctatgctctcagccaaggaacaacaatcagaggaaaacactcaaacggaacaatacacgataggtcccagtatcgcgccctgataagctggccactatcatcaccgcccacagtgtacaacagcagggtggaatgcattgggtggtcaagtactagttgccatgatggcaaatccaggatgtcaatatgtatatcaggaccaaacaacaatgcatctgcagtagtatggtacaacagaaggcctgttgcagaaattaacacatgggcccgaaacatactaagaacacaggaatctgaatgtgtatgccacaacggcgtatgcccagtagtgttcaccgatgggtctgccactggacctgcagacacaagaatatactattttaaagaggggaaaatattgaaatgggagtctctgactggaactgctaagcatattgaagaatgctcatgttacggggaacgaacaggaattacctgcacatgcaaggacaattggcagggctcaaatagaccagtgattcagatagatccagtagcaatgacacacactagtcagtatatatgcagtcctgttcttacagacaatccccgaccgaatgacccaaatataggtaagtgtaatgacccttatccaggtaataataacaatggagtcaagggattctcatacctggatggggctaacacttggctagggaggacaataagcacagcctcgaggtctggatacgagatgttaaaagtgccaaatgcattgacagatgatagatcaaagcccattcaaggtcagacaattgtattaaacgctgactggagtggttacagtggatctttcatggactattgggctgagggggactgctatcgagcgtgtttttatgtggaattgatacgtggaagacccaaggaggataaagtgtggtggaccagcaatagtatagtatcgatgtgttccagtacagaattcctgggacaatggaactggcctgatggggctaaaatagagtacttcctctaa

SEQ ID NO: 27 - protein, 462 aa, Influenza virus
MNPNQKILCTSATAIIIGAIAVLIGIANLGLNIGLHLKPSCNCSHSQPETINTSQTIINNYYNETNITNIQMEERTSRNFNNLTKGLCTINSWHIYGKDNAVRIGESSDVLVTREPYVSCDPDECRFYALSQGTIIRGKHSNGTIHDRSQYRALISWPLSSPPTVYNSRVECIGWSSTSCHDGKSRMSICISGPNNNASAWWYNRRPVAEINTWARNILRTQESECVCHNGVCPWFTDGSATGPADTRIYYFKEGKLKWESLTGTAKHIEECSCYGERTGITCTCKDNWQGSNRPVIQIDPVAMTHTSQYICSPVLTDNPRPNDPNIGKCNDPYPGNNNNGVKGFSYLDGANTWLGRTISTASRSGYEMLKVPNALTDDRSKPIQGQTIVLNADWSGYSGSFMDYWAEGDCYRACFYVELIRGRPKEDKVWWTSNSIVSMCSSTEFLGQWNWPDGAKIEYFL

(19) United States
(12) Patent Application Publication
(10) Pub. No.: US 2025/0034214 A1
(43) Pub. Date: Jan. 30, 2025

(54) INFLUENZA VIRUS REPLICATION FOR VACCINE DEVELOPMENT

(71) Applicant: Wisconsin Alumni Research Foundation (WARF), Madison, WI (US)

(72) Inventors: Yoshihiro Kawaoka, Middleton, WI (US); Gabriele Neumann, Madison, WI (US); Jihui Ping, Nanjing (CN)

(21) Appl. No.: 18/908,520

(22) Filed: Oct. 7, 2024

Related U.S. Application Data
(63) Continuation of application No. 17/813,200, filed on Jul. 18, 2022, now Pat. No. 12,122,807.
Application No. 17/813,200 is a continuation of application No. 16/865,194, filed on May 1, 2020, now Pat. No. 11,390,649.
(60) Provisional application No. 62/841,491, filed on May 1, 2019.

(51) Int. Cl. C07K 14/005; A61K 9/00; A61K 39/00; A61K 39/145; A61P 31/16; C12N 7/00

(52) U.S. Cl. CPC C07K 14/005; A61K 9/0019; A61K 9/007; A61K 39/145; A61P 31/16; C12N 7/00; A61K 2039/54; A61K 2039/544; C12N 2760/16121; C12N 2760/16122; C12N 2760/16134; C12N 2760/16152

(57) ABSTRACT

An isolated recombinant influenza virus is provided having PA, PB1, PB2, NP, NS, M, NA and HA viral segments, wherein the PB1 viral segment encodes a PB1 with a residue other than isoleucine at position 711 or the M viral segment encodes a M1 with a residue other than methionine at position 128, wherein the recombinant influenza virus has enhanced replication relative to a corresponding influenza virus having a PB1 viral segment that encodes a PB1 with an isoleucine at position 711 or having a M viral segment that encodes a M1 with methionine at position 128, as well as methods of making and using the virus.

### CROSS-REFERENCE TO RELATED APPLICATIONS

This application is a continuation of U.S. application Ser. No. 17/813,200, filed Jul. 18, 2022, which is a continuation of U.S. application Ser. No. 16/865,194, filed May 1, 2020, which claims the benefit of the filing date of U.S. application No. 62/841,491, filed on May 1, 2019, the disclosures of which are incorporated by reference herein.

### STATEMENT OF GOVERNMENT RIGHTS

This invention was made with government support under HHSN272201400008C awarded by the National Institutes of Health. The government has certain rights in the invention.

### INCORPORATION BY REFERENCE OF SEQUENCE LISTING

A Sequence Listing is provided herewith as an xml file, “2315414.xml” created on Mar. 7, 2023, and having a size of 64,341 bytes. The content of the xml file is incorporated by reference herein in its entirety.

### BACKGROUND

Influenza is a major respiratory disease in some mammals and is responsible for substantial morbidity and economic losses each year. In addition, influenza virus infections can cause severe systemic disease in some avian species, leading to death. The segmented nature of the influenza virus genome allows for reassortment of segments during virus replication in cells infected with two or more influenza viruses. The reassortment of segments, combined with genetic mutation and drift, can give rise to a myriad of divergent strains of influenza virus over time. The new strains exhibit antigenic variation in their hemagglutinin (HA) and/or neuraminidase (NA) proteins, and in particular the gene coding for the HA protein has a high rate of variability. The predominant current practice for the prevention of flu is vaccination. As the influenza HA protein is the major target antigen for the protective immune responses of a host to the virus and is highly variable, the isolation of influenza virus and the identification and characterization of the HA antigen in viruses associated with recent outbreaks is important for vaccine production. Based on prevalence and prediction, a vaccine is designed to stimulate a protective immune response against the predominant and expected influenza virus strains (Park et al., 2004).

There are three general types of influenza viruses, Type A, Type B and Type C, which are defined by the absence of serological crossreactivity between their internal proteins. Influenza Type A viruses are further classified into subtypes based on antigenic and genetic differences of their glycoproteins, the HA and NA proteins. Most of all the known HA and NA subtypes (H1 to H16 and N1 to N9) have been isolated from aquatic birds, which are thought to act as a natural reservoir for influenza. The H1N1 pandemic virus caused a pandemic in 2009. The first vaccine candidates tested in 2009 did not grow to high titers, demonstrating the need to develop vaccine virus backbones that confer efficient replication to vaccine virus candidates.

### SUMMARY

Several strategies were employed (including random mutagenesis and the comprehensive testing of potentially growth-enhancing mutations) to develop influenza A/Puerto Rico/8/34 (H1N1; the strain commonly used for the generation of inactivated influenza vaccines) viruses that replicate to high titers in cultured cells and/or embryonated chicken eggs. A number of growth-enhancing mutations were identified that increase the yield of influenza vaccine viruses. Individual growth-enhancing residues in an influenza virus polypeptide may be combined with one or more other growth-enhancing residues in the same influenza virus polypeptide, or with one or more other growth-enhancing residues in other influenza virus polypeptide(s), as well with growth-enhancing nucleotides in viral non-coding regions, e.g., promoter sequences. For example, one or more growth-enhancing residues in a polymerase protein, for instance, 1, 2, 3, 4, 5, 6, 7 or more, growth-enhancing residues in PB2, 1, 2, 3, 4, 5, 6, 7 or more, e.g., up to 12, 13, 14 or 15, growth-enhancing residues in PB1, 1, 2, 3, or 4 or more growth-enhancing residues in PA, or 1, 2, 3, or 4 growth-enhancing residues in NP, 1, 2, 3, or 4 growth-enhancing residues in M, e.g., 1, 2, or 3 growth-enhancing residues in M1, 1, 2, or 3 growth-enhancing residues in NS1, or any combination of growth-enhancing residues or nucleotides in viral non-coding, e.g., promoter sequences, may be combined when preparing influenza virus, e.g., for a vaccine, to enhance viral titers. In one embodiment, growth-enhancing nucleotides in viral promoter sequences may be introduced to a viral segment, or when present in a viral segment may be selected for inclusion in an influenza virus. In one embodiment, growth-enhancing residues in HA and/or in NA may be introduced into, or when present in a HA or NA selected for inclusion in, a HA viral segment or a NA viral segment in an influenza virus. In one embodiment, the one or more growth-enhancing residues may enhance viral growth by at least 1.2, 2, 2.8, 4, 3, 5, 6, 8, 10, 100, or 200 fold or more

Mutations that increase the replicative ability of viruses in cell culture and/or embryonated chicken eggs are useful to amplify influenza viruses and to establish robust influenza vaccine platforms. Currently, most influenza vaccines are generated in embryonated chicken eggs. Influenza vaccines generated in MDCK cells are now approved for human use in the U.S. and in Europe, and influenza vaccines derived from Vero cells are approved for human use in Europe. Virus libraries possessing random mutations in the ‘internal’ viral genes (i.e., all viral genes except those encoding the viral surface glycoproteins HA and NA) of a vaccine virus isolate, e.g., UW-PR8 (see Example 1), were generated and passaged in MDCK cells. The identified mutations result in higher virus titers in MDCK cells (and may also increase virus titers in Vero cells and/or embryonated chicken eggs), allowing more efficient influenza virus growth and more cost-effective vaccine production. Moreover, previously described mutations increased the replicative ability of UW-PR8 vaccine backbone virus. In addition to mutations in the coding regions of the six internal viral segments, mutations in non-coding regions were observed to increase viral titers, including promoter mutations, for instance, C-to-U mutations at position 4 from the 3′ end of the PB2, PB1, and/or PA vRNA segments. The resulting sequences may be also codon-usage optimized, e.g., optimized for expression in mammalian cells such as canine cells or primate cells, or avian cells, e.g., chicken embryos. The mutations can be used in various combinations, with results influenced by the cell line (or egg) in use and the desired level of improvement in the replication of the virus.

The invention provides isolated recombinant, e.g., reassortant, influenza viruses with selected amino acid residues at one or more specified positions in one or more viral segments for PA, PB1, PB2, NP, M (encoding M1 and M2 proteins), and/or NS (encoding NS1 and NS2 proteins), e.g., in selected amino acid residues at specified positions of PB1, PB2 and NS1; PA, PB1, PB2, NP and NS1; PB1, PB2, NP, M1, and NS1; PA, PB1, PB2, NP and NS1; or PA, PB1, PB2, NP, M1, and NS1, and including HA and NA genes/proteins of interest, e.g., from annual and pandemic strains, which viruses are produced more efficiently and cost-effectively via cell culture (in MDCK or Vero cells) or in embryonated chicken eggs than parental virus. As used herein, a “viral segment” in a virus means an influenza vRNA sequence and a “viral segment” in a transcription cassette for production of a viral segment means a sequence that when introduced into a cell or appropriate cell-free system and transcribed, yields influenza vRNA or cRNA. Recombinant virus may also be obtained by generating influenza viral plus-sense cRNA from a transcription cassette.

In one embodiment, the isolated recombinant influenza virus has PA, PB1, PB2, NP, NS, and M viral segments from a first influenza virus isolate, a heterologous or chimeric influenza virus NA viral segment, and a heterologous or chimeric HA viral segment, wherein the PB1 viral segment encodes a PB1 with a residue other than isoleucine at position 711 or the M viral segment encodes a M1 with a residue other than methionine at position 128, wherein the recombinant influenza virus has enhanced replication, e.g., in MDCK cells, Vero cells or eggs, relative to a corresponding influenza virus having a PB1 viral segment that encodes a PB1 with an isoleucine at position 711 or having a M viral segment that encodes a M1 with methionine at position 128. In one embodiment, the PB1 viral segment encodes a PB1 with a residue other than isoleucine at position 711 and the M viral segment encodes a M1 with a residue other than methionine at position 128. In one embodiment, the PA viral segment encodes a PA with a residue other than phenylalanine at position 105, a residue other than lysine at position 142, a residue other than serine at position 149, a residue other than serine at position 225, a residue other than lysine at position 356, a residue other than threonine at position 357, a residue other than arginine at residue 401, or a residue other than isoleucine at position 550, or any combination thereof. In one embodiment, the PB1 viral segment further encodes a PB1 with a residue other than methionine at position 40, a residue other than glutamic acid at position 112, a residue other than glycine at position 180, or a residue other than glutamine at residue 247, or any combination thereof. In one embodiment, the PB2 viral segment encodes a PB2 a residue other than methionine at position 202, a residue other than phenylalanine at position 323, or a residue other than isoleucine at position 504, or any combination thereof. In one embodiment, the NP viral segment encodes a NP with a residue other than arginine at position 74, a residue other than isoleucine at position 116, or a residue other than asparagine at position 417, or any combination thereof. In one embodiment, the NS viral segment encodes a NS1 with a residue other than alanine at position 30, a residue other than lysine at position 55, or a residue other than arginine at position 118, or any combination thereof. In one embodiment, the recombinant virus has a V, A, L, G, S, T, C, or M at position 711 in PB1, has an A, L, G, V, S, T, I, or C at position 40 in PB1, has an A, L, G, V, S, T, I, or C at position 112 in PB1, has an A, L, W, Y, F, V, S, T, I, or C at position 180 in PB1, has a H, R, K, D, E, D or E at position 247 in PB1, or any combination thereof. In one embodiment, the recombinant virus has a K, H, D, E, Q or N at position 74 in NP, has a L, V, G, A, S, C or T at position 116 in NP, has a D, E, Q, K, or H at position 417 in NP, or any combination thereof. In one embodiment, the recombinant virus has a P, W, F, L, I, V, G, S, C or T at position 30 in NS1, has E, D, Q, or N at position 55, has a K, H, D, E, Q, or N at position 118 in NS1, has a L, I, V, G, A, S, C or T at position 128 in M1, or any combination thereof. In one embodiment, the recombinant virus has a C, L, I, V, G, A, or T at position 142 in PA, has a C, L, I, V, G, A, or T at position 225 in PA, has a K, H, D, E, N or Q at position 401 in PA, or any combination thereof. In one embodiment, the recombinant virus has a V, A, L, G, S, T, C, or M at position 504 in PB2, has a A, L, G, S, T, I, or C at position 202 in PB2, has a A, L, G, S, T, I, or C at position 323 in PB2, or any combination thereof. In one embodiment, the recombinant virus has a V, A, L, G, S, T, C, or M at position 711 in PB1, has a A, L, G, V, S, T, I, or C at position 40 in PB1, has a A, L, W, Y, F, V, S, T, I, or C at position 180 in PB1; has a L, V, G, A, S, C or T at position 116 in NP; has a P, W, F, L, I, V, G, S, C or T at position 30 in NS1, has a K, H, D, E, Q, or N at position 118 in NS1; has a L, I, V, G, A, S, C or T at position 128 in M1; has a K, H, D, E, N or Q at position 401 in PA; has a V, A, L, G, S, T, C, or M at position 504 in PB2; or any combination thereof. In one embodiment, the recombinant virus has 504V in PB2. In one embodiment, the recombinant virus has 74K, 116L or 417D in NP. In one embodiment, the recombinant virus has 30P, 55E or 118K in NS1. In one embodiment, the recombinant virus has 225C or 401K in PA. In one embodiment, the recombinant virus of has a V, A, L, G, S, T, C, or M at position 711 in PB1. In one embodiment, the recombinant virus has a L, I, V, G, A, S, C or T at position 128 in M1. In one embodiment, the recombinant virus has a U at position 4 in the viral segment for any one of PB1, PB2 or PA. In one embodiment, the recombinant virus has a U at position 4 in the viral segment for PB1, PB2 and PA. In one embodiment, the PA, PB1, PB2, NP, NS, and M viral segments comprise sequences for at least one of the following: a PB1 having the amino acid sequence encoded by SEQ ID NO:2 or PB1 with at least 95% amino acid sequence identity to the PB1 encoded by SEQ ID NO:2; a PB2 having the amino acid sequence encoded by SEQ ID NO:3 or PB2 with at least 95% amino acid sequence identity to the PB2 encoded by SEQ ID NO:3; a PA having the amino acid sequence encoded by SEQ ID NO:1 or PA with at least 95% amino acid sequence identity to the PA encoded by SEQ ID NO:1; a NP having the amino acid sequence encoded by SEQ ID NO:4 or NP with at least 95% amino acid sequence identity to the NP encoded by SEQ ID NO:4; a M1 and/or M2 having the amino acid sequence encoded by SEQ ID NO:5 or M1 and/or M2 with at least 95% amino acid sequence identity to the M1 and/or M2 encoded by SEQ ID NO:5; or a NS1 and/or Ns2 having the amino acid sequence encoded by SEQ ID NO:6 or NS1 and/or NS2 with at least 95% amino acid sequence identity to the NS encoded by SEQ ID NO:6 or wherein the PA, PB1, PB2, NP, NS, and M viral segments comprise sequences for at least one of the following: a PB1 having the amino acid sequence encoded by SEQ ID NO:10 or PB1 with at least 95% amino acid sequence identity to the PB1 encoded by SEQ ID NO:10; a PB2 having the amino acid sequence encoded by SEQ ID NO:11 or PB2 with at least 95% amino acid sequence identity to the PB2 encoded by SEQ ID NO:11; a PA having the amino acid sequence encoded by SEQ ID NO:12 or PA with at least 95% amino acid sequence identity to the PA encoded by SEQ ID NO:12; a NP having the amino acid sequence encoded by SEQ ID NO:13 or NP with at least 95% amino acid sequence identity to the NP encoded by SEQ ID NO:13; a M1 and/or M2 having the amino acid sequence encoded by SEQ ID NO:14 or M1 and/or M2 with at least 95% amino acid sequence identity to the M1 and/or M2 encoded by SEQ ID NO:14; or a NS1 and/or NS2 having the amino acid sequence encoded by SEQ ID NO:15 or NS1 and/or NS2 with at least 95% amino acid sequence identity to the NS1 and/or NS2 encoded by SEQ ID NO:15. In one embodiment, two of the PA, PB1, PB2, NP, NS, and M viral segments have at least one of the alterations disclosed herein. In one embodiment, three of the PA, PB1, PB2, NP, NS, and M viral segments have at least one of the alterations disclosed herein. In one embodiment, four of the PA, PB1, PB2, NP, NS, and M viral segments have at least one of the alterations disclosed herein. In one embodiment, five of the PA, PB1, PB2, NP, NS, and M viral segments have at least one of the alterations disclosed herein. In one embodiment, the PA, PB1, PB2, NP, NS, and M viral segments have at least one of the alterations disclosed herein.

Also provided is a method to prepare influenza virus, comprising: contacting a cell with: a vector for vRNA production comprising a promoter operably linked to an influenza virus PA DNA linked to a transcription termination sequence, a vector for vRNA production comprising a promoter operably linked to an influenza virus PB1 DNA linked to a transcription termination sequence, a vector for vRNA production comprising a promoter operably linked to an influenza virus PB2 DNA linked to a transcription termination sequence, a vector for vRNA production comprising a promoter operably linked to an influenza virus HA DNA linked to a transcription termination sequence, a vector for vRNA production comprising a promoter operably linked to an influenza virus NP DNA linked to a transcription termination sequence, a vector for vRNA production comprising a promoter operably linked to an influenza virus NA DNA linked to a transcription termination sequence, a vector for vRNA production comprising a promoter operably linked to an influenza virus M DNA linked to a transcription termination sequence, and a vector for vRNA production comprising a promoter operably linked to an influenza virus NS DNA linked to a transcription termination sequence, wherein the PB1, PB2, PA, NP, NS, and M DNAs in the vectors for vRNA production are from one or more influenza vaccine virus isolates, wherein the NA DNA in the vector for vRNA production of NA has sequences for a heterologous NA, and wherein the HA DNA in the vector for vRNA production of HA has sequences for a heterologous HA, wherein the PB1 DNA encodes a PB1 with a residue other than isoleucine at position 711 or the M DNA encodes a M1 with a residue other than methionine at position 128; and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2; in an amount effective to yield infectious influenza virus. In one embodiment, the cell is an avian or a mammalian cell. In one embodiment, the cell is a Vero cell, a human cell or a MDCK cell. The cell is in an avian egg.

Further provided is a method of using the recombinant virus. In one embodiment, an avian or a mammal is administered an amount of the virus effective to induce an immune response.

In one embodiment, the invention provides an isolated recombinant reassortant influenza virus having six “internal” viral segments from a vaccine influenza virus with two or more of the selected amino acid residues at specified positions described herein, and a NA viral segment selected from a first influenza virus isolate, and a HA viral segment from the same isolate or a different isolate.

In one embodiment, the influenza virus of the invention is a recombinant influenza virus having a particular amino acid residue at specified positions, as disclosed herein, in one, two, three or more of PA, PB1, PB2, NP, M1 and/or NS1 and having an amino acid sequence with at least 80%, e.g., 90%, 92%, 95%, 97%, 98%, or 99%, including any integer between 80 and 99, contiguous amino acid sequence identity to a corresponding polypeptide encoded by one of SEQ ID Nos. 1-6 or 10-15. The residue other than the specified residue may be conservative substitution. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chain is cysteine and methionine. In one embodiment, conservative amino acid substitution groups are: threonine-valine-leucine-isoleucine-alanine or valine-leucine-isoleucine-alanine; phenylalanine-tyrosine; lysine-arginine; alanine-valine; glutamic acid-aspartic acid; and asparagine-glutamine.

In one embodiment, the influenza virus of the invention is a recombinant influenza virus having a particular amino acid residue at specified positions in PA, PB1, PB2, NP, M1, and/or NS1 and an amino acid sequence with at least 80%, e.g., 90%, 92%, 95%, 97%, 98%, or 99%, including any integer between 80 and 99, contiguous amino acid sequence identity to a corresponding polypeptide encoded by one of SEQ ID Nos. 1-6 or 10-15, e.g., a polypeptide with a residue other than R401 in PA, M40 or G180 in PB1, I504 in PB2, I116 in NP, and/or A30 or R118 in NS1, or any combination thereof, as well as a residue other than I711 in PB1 or M128 in M1.

In one embodiment, the influenza virus of the invention is a recombinant influenza virus having a particular amino acid residue at specified positions in PA, PB1, PB2, NP, M1, and/or NS1 and an amino acid sequence with at least 80%, e.g., 90%, 92%, 95%, 97%, 98%, or 99%, including any integer between 80 and 99, contiguous amino acid sequence identity to a corresponding polypeptide encoded by one of SEQ ID Nos. 1-6 or 10-15, e.g., a polypeptide with a residue other than R401 in PA, M40 or G180 in PB1, I504 in PB2, I116 in NP, and/or A30 or R118 in NS1, or any combination thereof, as well as a residue other than I711 in PB1 and M128 in M1.

In one embodiment, the influenza virus of the invention is a recombinant influenza virus having a particular amino acid residue at specified positions in PA, PB1, PB2, NP, M1, and/or NS1 and an amino acid sequence with at least 80%, e.g., 90%, 92%, 95%, 97%, 98%, or 99%, including any integer between 80 and 99, contiguous amino acid sequence identity to a corresponding polypeptide encoded by one of SEQ ID Nos. 1-6 or 10-15, e.g., a polypeptide with a residue that is a non-conservative substitution relative to R401 in PA, M40 or G180 in PB1, I504 in PB2, I116 in NP, and/or A30 or R118 in NS1, or any combination thereof, as well as a conservative substitution relative to 1711 in PB1 or a conservative substitution relative to M128 in M1.

In one embodiment, the influenza virus of the invention is a recombinant influenza virus having a particular amino acid residue at specified positions in PA, PB1, PB2, NP, M1, and/or NS1 and an amino acid sequence with at least 80%, e.g., 90%, 92%, 95%, 97%, 98%, or 99%, including any integer between 80 and 99, contiguous amino acid sequence identity to a corresponding polypeptide encoded by one of SEQ ID Nos. 1-6 or 10-15, e.g., a polypeptide with a residue that is a non-conservative substitution relative to R401 in PA, M40 or G180 in PB1, I504 in PB2, I116 in NP, and/or A30 or R118 in NS1, or any combination thereof, as well as a conservative substitution relative to I711 in PB1 and a conservative substitution relative to M128 in M1.

In one embodiment, the influenza virus of the invention is a recombinant influenza virus having a particular amino acid residue at specified positions in PA, PB1, PB2, NP, M1, and/or NS1 and an amino acid sequence with at least 80%, e.g., 90%, 92%, 95%, 97%, 98%, or 99%, including any integer between 80 and 99, contiguous amino acid sequence identity to a corresponding polypeptide encoded by one of SEQ ID Nos. 1-6 or 10-15, e.g., a polypeptide with a residue that is a non-conservative substitution relative to R401 in PA, M40 or G180 in PB1, I504 in PB2, I116 in NP, and/or A30 or R118 in NS1, or any combination thereof, as well as a conservative substitution relative to I711 in PB1 or a non-conservative substitution relative to M128 in M1.

In one embodiment, the influenza virus of the invention is a recombinant influenza virus having a particular amino acid residue at specified positions in PA, PB1, PB2, NP, M1, and/or NS1 and an amino acid sequence with at least 80%, e.g., 90%, 92%, 95%, 97%, 98%, or 99%, including any integer between 80 and 99, contiguous amino acid sequence identity to a corresponding polypeptide encoded by one of SEQ ID Nos. 1-6 or 10-15, e.g., a polypeptide with a residue that is a non-conservative substitution relative to R401 in PA, M40 or G180 in PB1, I504 in PB2, I116 in NP, and/or A30 or R118 in NS1, or any combination thereof, as well as a conservative substitution relative to I711 in PB1 and a non-conservative substitution relative to M128 in M1.

In one embodiment, the influenza virus of the invention is a recombinant influenza virus having a particular amino acid residue at specified positions in PA, PB1, PB2, NP, M1, and/or NS1 and an amino acid sequence with at least 80%, e.g., 90%, 92%, 95%, 97%, 98%, or 99%, including any integer between 80 and 99, contiguous amino acid sequence identity to a corresponding polypeptide encoded by one of SEQ ID Nos. 1-6 or 10-15, e.g., a polypeptide with a conservative residue other than R401 in PA, a non-conservative residue other than M40 or G180 in PB1, a conservative residue other than I504 in PB2 and I116 in NP, a non-conservative residue at A30 or a conservative residue at R118 in NS1, or any combination thereof, as well as a conservative residue other than I711 in PB1 or a non-conservative residue other than M128 in M1.

In one embodiment, the influenza virus of the invention is a recombinant influenza virus having a particular amino acid residue at specified positions in PA, PB1, PB2, NP, M1, and/or NS1 and an amino acid sequence with at least 80%, e.g., 90%, 92%, 95%, 97%, 98%, or 99%, including any integer between 80 and 99, contiguous amino acid sequence identity to a corresponding polypeptide encoded by one of SEQ ID Nos. 1-6 or 10-15, e.g., a polypeptide with a conservative residue other than R401 in PA, a non-conservative residue other than M40 or G180 in PB1, a conservative residue other than I504 in PB2 and I116 in NP, a non-conservative residue at A30 or a conservative residue at R118 in NS1, or any combination thereof, as well as a conservative residue other than I711 in PB1 and a non-conservative residue other than M128 in M1.

Also included are any combination of the selected amino acid residues at specified positions described herein.

Viral segments for of PA, PB1, PB2, NP, M and/or NS that have the residues at the specified positions may be combined with a viral segment for HA, e.g., H1, H2, H3, H4, H5, H6, H7, H8, H9, H10, H11, H12, H13, H14, H15, H16, or H17 and a viral segment for NA, e.g., N1, N2, N3, N4, N5, N6, N7, N8, N9, or N10, and any combination of HA and NA, to provide the reassortant vaccine viruses of the invention. In one embodiment, the HA is H1, H5 or H7. In one embodiment the NA is N1 or N9. In one embodiment, the HA viral segment in the reassortant virus is heterologous to the viral segments for PA, PB1, PB2, NP, M and NS. In one embodiment, the NA viral segment in the reassortant virus is heterologous to the viral segments for PA, PB1, PB2, NP, M and NS. In one embodiment, the HA viral segment in the reassortant virus has viral segments for PA, PB1, PB2, NP, M and NS from one influenza virus isolate or strain (“parent”), or a variant thereof, e.g., one with viral segments encoding influenza virus proteins with at least 95%, 96%, 97%, 98%, 99%, or 99.5% amino acid sequence identity, or having 1, 2, 5, 10, or 20 substitutions relative, to sequences in a parent influenza virus isolate or strain. In one embodiment, the parent strain has viral segments with sequences corresponding to SEQ ID Nos. 1-6 or 10-15. In one embodiment, the HA viral segment in the reassortant virus is a chimeric HA viral segment, e.g., a chimera of heterologous HA ectodomain sequences linked to HA signal peptide sequences and/or HA transmembrane domain sequences from the HA viral segment of the parent isolate or strain, or variant thereof. In one embodiment, the NA viral segment in the isolated recombinant virus is a chimeric NA viral segment e.g., a chimera of heterologous NA ectodomain sequences linked to NA transmembrane domain sequences from the NA viral segment of the parent isolate or strain, or variant thereof, and/or stalk sequences from the parent isolate or strain, or variant thereof. In one embodiment, the NA viral segment in the isolated recombinant virus is a chimeric NA viral segment e.g., a chimera of heterologous NA ectodomain sequences linked to NA transmembrane domain sequences from the NA viral segment of the parent isolate or strain, or variant thereof, and/or stalk sequences from a second isolate or strain, or variant thereof. In one embodiment, the isolated recombinant virus has a heterologous HA viral segment, a heterologous NA viral segment, a chimeric HA viral segment, a chimeric NA viral segment, or any combination thereof. The nucleic acid sequences employed to prepare vRNA may be ones that introduce the residues at the specified positions via recombinant methodology or may be selected as having the residues at the specified positions.

A/Puerto Rico/8/34 (H1N1), “PR8,” virus serves as the genetic backbone for generation of inactivated influenza vaccines. Occasionally, vaccine strains based on PR8 backbone replicate to relatively low titers in eggs and cell culture resulting in delayed vaccine production and vaccine shortage. To determine if high yield vaccine strain backbones for propagation in MDCK cells, chicken eggs and Vero cells can be prepared to supply the demand of seasonal flu and highly pathogenic pandemic viruses, various mutagenesis strategies were employed. For example, PR8 backbone random mutant libraries were screened for high replicative mutants, e.g., by introducing random mutations to internal PR8 genes by error prone PCR, introducing mutations that confer high replication and high polymerase activity, and optimizing PR8 internal gene via codon bias.

As described herein, virus libraries with random mutations related to an influenza virus isolate useful as a vaccine virus (e.g., A/Puerto Rico/8/34, “PR8,” including a specific isolate such as UW-PR8) to carry heterologous viral segments for NA and/or HA, were serially passaged in MDCK cells, e.g., about 10-12-times although fewer passages may be employed, to obtain virus with enhanced replication in those cells. In one embodiment, viruses obtained after serial passage which have enhanced replication, have titers that are at least 1 or 2 logs higher than viruses that were not serially passaged. In one embodiment, viruses obtained after serial passage had substitutions in two or more internal viral segments relative to the parent virus.

Thus, for vaccine viruses that are to be grown or passaged in cells in culture, e.g., MDCK or Vero cells or eggs, selection of sequences with, or replacement of, the disclosed residues at the specified positions in one or more of PA, PB1, PB2, NP, and/or NS1, that confer enhanced growth of the virus in cultured cells when employed with HA and NA sequences of interest, can result in significantly higher viral titers. Thus, the invention provides a method to select for influenza viruses with enhanced replication in cell culture. The method includes providing cells suitable for influenza vaccine production; serially culturing one or more influenza virus isolates in the cells; and isolating serially cultured virus with enhanced growth relative to the one or more isolates prior to serial culture. In one embodiment, the cells are canine or primate, e.g., human or monkey, cells.

The invention provides a plurality of influenza virus vectors of the invention, e.g., those useful to prepare reassortant viruses including 6:1:1 reassortants, 6:2 reassortants and 7:1 reassortants. A 6:1:1 reassortant within the scope of the present invention is an influenza virus with 6 internal viral segments from a vaccine virus, a NA viral segment from a different (second) viral isolate, and a HA viral segment from a third isolate; a 6:2 reassortant within the scope of the present invention is an influenza virus with 6 internal viral segments from a vaccine virus, and a NA viral segment and a HA viral segment from a different (second) viral isolate; and a 7:1 reassortant within the scope of the present invention is an influenza virus with 6 internal viral segments and a NA viral segment from a vaccine virus, and a HA viral segment from a different viral source than the vaccine virus, or an influenza virus with 6 internal viral segments and a HA viral segment from the vaccine virus, and a NA viral segment is from a different viral source than the vaccine virus.

In one embodiment of the invention, the plurality includes vectors for vRNA production selected from a vector comprising a promoter operably linked to an influenza virus PA DNA linked to a transcription termination sequence, a vector comprising a promoter operably linked to an influenza virus PB1 DNA linked to a transcription termination sequence, a vector comprising a promoter operably linked to an influenza virus PB2 DNA linked to a transcription termination sequence, a vector comprising a promoter operably linked to an influenza virus HA DNA linked to a transcription termination sequence, a vector comprising a promoter operably linked to an influenza virus NP DNA linked to a transcription termination sequence, a vector comprising a promoter operably linked to an influenza virus NA DNA linked to a transcription termination sequence, a vector comprising a promoter operably linked to an influenza virus M DNA linked to a transcription termination sequence, and a vector comprising a operably linked to an influenza virus NS DNA linked to a transcription termination sequence. In one embodiment, the DNAs for vRNA production of PB1, PB2, PA, NP, M, and NS, have sequences from an influenza virus that replicates to high titers in cultured mammalian cells such as MDCK cells, Vero cells or PER.C6® cells and also optionally embryonated eggs, and/or from a vaccine virus, e.g., one that does not cause significant disease in humans. The DNA for vRNA production of NA may be from any NA, e.g., any of N1-N1, and the DNA for vRNA production of HA may be from any HA, e.g., H1-H18. In one embodiment, the DNAs for vRNA production may be for an influenza B or C virus. The DNAs for vRNA production of NA and HA may be from different strains or isolates (6:1:1 reassortants) or from the same strain or isolate (6:2 reassortants), or the NA may be from the same strain or isolate as that for the internal genes (7:1 reassortant). The plurality also includes vectors for mRNA production selected from a vector encoding influenza virus PA, a vector encoding influenza virus PB1, a vector encoding influenza virus PB2, and a vector encoding influenza virus NP, and optionally one or more vectors encoding NP, NS, M, e.g., M1 and M2, HA or NA. The vectors encoding viral proteins may further include a transcription termination sequence.

Viruses that may provide the internal genes for reassortants within the scope of the invention include viruses that have high titers in MDCK cells, e.g., titers of at least about 10⁵ PFU/mL, e.g., at least 10⁶ PFU/mL, 10⁷ PFU/mL or 10⁸ PFU/mL; high titers in embryonated eggs, e.g., titers of at least about 10⁷ EID₅₀/mL, e.g., at least 10⁸ EID₅₀/mL, 10⁹ EID₅₀/mL or 10¹⁰ EID₅₀/mL; high titers in cells such as MDCK cells, e.g., titers of at least about 10⁷ PFU/mL, e.g., at least 10⁸ PFU/mL, or high titers in two of more of those host cells.

In one embodiment, the titers of the reassortant viruses of the invention in cells such as MDCK cells or Vero cells may be over 1 log, 2 logs, 3 logs, or greater, than titers of the corresponding virus without particular residues at the specified positions.

Other reassortants with internal genes from other PR8 isolates or vaccine viruses may be employed in recombinant reassortant viruses of the invention. In particular, 5:1:2 reassortants having UW-PR8 PB1, PB2, PA, NP, and M (“5”) and PR8(Cam) NS (“1”); 6:1:1 reassortants having UW-PR8 NA, PB1, PB2, PA, NP, and M (“6”) and PR8(Cam) NS (“1”); and 7:1 reassortants having UW-PR8 PB1, PB2, PA, NP, M, NA, and NS (“7”) may be employed.

In one embodiment, the DNAs for the internal genes for PB1, PB2, PA, NP, M, and NS encode proteins with substantially the same activity as a corresponding polypeptide encoded by one of SEQ ID NOs:1-6 or 10-15. As used herein, “substantially the same activity” includes an activity that is about 0.1%, 1%, 10%, 30%, 50%, 90%, e.g., up to 100% or more, or detectable protein level that is about 80%, 90% or more, the activity or protein level, respectively, of the corresponding full-length polypeptide. In one embodiment, the nucleic acid a sequence encoding a polypeptide which is substantially the same as, e.g., having at least 80%, e.g., 90%, 92%, 95%, 97%,98%, or 99%, including any integer between 80 and 99, contiguous amino acid sequence identity to, a polypeptide encoded by one of SEQ ID NOs:1-6 or 10-15. In one embodiment, the isolated and/or purified nucleic acid molecule comprises a nucleotide sequence which is substantially the same as, e.g., having at least 50%, e.g., 60%, 70%, 80% or 90%, including any integer between 50 and 100, or more contiguous nucleic acid sequence identity to one of SEQ ID NOs:1-6 or 10-15 and, in one embodiment, also encodes a polypeptide having at least 80%, e.g., 90%, 92%, 95%, 97%,98%, or 99%, including any integer between 80 and 99, contiguous amino acid sequence identity to a polypeptide encoded by one of SEQ ID NOs:1-6 or 10-15. In one embodiment, the influenza virus polypeptide has one or more, for instance, 2, 5, 10, 15, 20 or more, conservative amino acids substitutions, e.g., conservative substitutions of up to 10% or 20% of 2, 5, 10, 15, 20 or more, of a combination of conservative and non-conservative amino acids substitutions, e.g., conservative substitutions of up to 10% or 20% of the residues, or relative to a polypeptide encoded by one of SEQ IS NOs:1-6 or 10-15, and has a characteristic residue in two or more of PA, PB1, PB2, NP, M1, and/or NS1 the residues, relative to a polypeptide encoded by one of SEQ ID NOs:1-6 or 10-15, and has a characteristic residue, as described herein, in two or more of the viral segments for PA, PB1, PB2, NP, M, and/or NS, such as in PA and PB1 viral segments, PB1 and PB2 viral segments, PA, PB1 and PB2 viral segments, or in PA, PB1, PB2 and NP viral segments, or in PA, PB1, PB2 and M viral segments. In one embodiment, the influenza virus polypeptide has one or more, for instance, 2, 3, 4, 5, 6, 7 or 8 conservative and/or nonconservative amino acid substitutions, relative to a polypeptide encoded by one of SEQ ID NOs:1-6 or 10-15, at positions including but not limited to 401 in PA, 40 or 180 and 711 in PB1, 504 in PB2, 116 in NP, 30 or 118 in NS1, or 128 in M1, or any combination thereof.

The invention thus includes the use of isolated and purified vectors or plasmids, which express or encode influenza virus proteins, or express or encode influenza vRNA, both native and recombinant vRNA. The vectors comprise influenza cDNA, e.g., influenza A (e.g., any influenza A gene including any of the 18 HA or 11 NA subtypes), B or C DNA (see Fields Virology (Fields et al. (eds.), Lippincott, Williams and Wickens (2006), which is specifically incorporated by reference herein). Any suitable promoter or transcription termination sequence may be employed to express a protein or peptide, e.g., a viral protein or peptide, a protein or peptide of a nonviral pathogen, or a therapeutic protein or peptide.

A composition or plurality of vectors of the invention may also comprise a heterologous gene or open reading frame of interest, e.g., a foreign gene encoding an immunogenic peptide or protein useful as a vaccine or in gene replacement, for instance, may encode an epitope useful in a cancer therapy or vaccine, or a peptide or polypeptide useful in gene therapy. When preparing virus, the vector or plasmid comprising the gene or cDNA of interest may substitute for a vector or plasmid for an influenza viral gene or may be in addition to vectors or plasmids for all influenza viral genes. Thus, another embodiment of the invention comprises a composition or plurality of vectors as described above in which one of the vectors is replaced with, or further comprises, 5′ influenza virus sequences optionally including 5′ influenza virus coding sequences or a portion thereof, linked to a desired nucleic acid sequence, e.g., a desired cDNA, linked to 3′ influenza virus sequences optionally including 3′ influenza virus coding sequences or a portion thereof. In one embodiment, the desired nucleic acid sequence such as a cDNA is in an antisense (antigenomic) orientation. The introduction of such a vector in conjunction with the other vectors described above to a host cell permissive for influenza virus replication results in recombinant virus comprising vRNA corresponding to the heterologous sequences of the vector.

The promoter in a vector for vRNA production may be a RNA polymerase I promoter, a RNA polymerase II promoter, a RNA polymerase III promoter, a T7 promoter, or a T3 promoter, and optionally the vector comprises a transcription termination sequence such as a RNA polymerase I transcription termination sequence, a RNA polymerase II transcription termination sequence, a RNA polymerase III transcription termination sequence, or a ribozyme. Ribozymes within the scope of the invention include, but are not limited to, tetrahymena ribozymes, RNase P, hammerhead ribozymes, hairpin ribozymes, hepatitis ribozyme, as well as synthetic ribozymes. In one embodiment, the RNA polymerase I promoter is a human RNA polymerase I promoter.

The promoter or transcription termination sequence in a vRNA or virus protein expression vector may be the same or different relative to the promoter or any other vector. In one embodiment, the vector or plasmid which expresses influenza vRNA comprises a promoter suitable for expression in at least one particular host cell, e.g., avian or mammalian host cells such as canine, feline, equine, bovine, ovine, or primate cells including human cells, or for expression in more than one host.

In one embodiment, at least one vector for vRNA comprises a RNA polymerase II promoter linked to a ribozyme sequence linked to viral coding sequences linked to another ribozyme sequences, optionally linked to a RNA polymerase II transcription termination sequence. In one embodiment, at least 2, e.g., 3, 4, 5, 6, 7 or 8, vectors for vRNA production comprise a RNA polymerase II promoter, a first ribozyme sequence, which is 5′ to a sequence corresponding to viral sequences including viral coding sequences, which is 5′ to a second ribozyme sequence, which is 5′ to a transcription termination sequence. Each RNA polymerase II promoter in each vRNA vector may be the same or different as the RNA polymerase II promoter in any other vRNA vector. Similarly, each ribozyme sequence in each vRNA vector may be the same or different as the ribozyme sequences in any other vRNA vector. In one embodiment, the ribozyme sequences in a single vector are not the same.

In one embodiment, the invention provides a plurality of influenza virus vectors for a reassortant, comprising a vector for vRNA production comprising a promoter operably linked to an influenza virus PA DNA linked to a transcription termination sequence, a vector for vRNA production comprising a promoter operably linked to an influenza virus PB1 DNA linked to a transcription termination sequence, a vector for vRNA production comprising a promoter operably linked to an influenza virus PB2 DNA linked to a transcription termination sequence, a vector for vRNA production comprising a promoter operably linked to an influenza virus HA DNA linked to a transcription termination sequence, a vector for vRNA production comprising a promoter operably linked to an influenza virus NP DNA linked to a transcription termination sequence, a vector for vRNA production comprising a promoter operably linked to an influenza virus NA DNA linked to a transcription termination sequence, a vector for vRNA production comprising a promoter operably linked to an influenza virus M DNA linked to a transcription termination sequence, and a vector for vRNA production comprising a promoter operably linked to an influenza virus NS cDNA linked to a transcription termination sequence, wherein the DNAs for PB1, PB2, PA, NP, NS, and M are from one or more influenza vaccine seed viruses and contain two or more of the characteristic residues at the specified position(s); and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2. In one embodiment, at least one vector comprises sequences corresponding to those encoding PB1, PB2, PA, NP, M, or NS, or a portion thereof, having substantially the same activity as a corresponding polypeptide encoded by one of SEQ ID NOs:1-6 or 10-15, e.g., a sequence encoding a polypeptide with at least 80%, e.g., 85%, 90%, 92%, 95%, 98%, 99% or 100%, including any integer between 80 and 100, amino acid identity to a polypeptide encoded by one of SEQ ID NOs:1-6 or 10-15. Optionally, two vectors may be employed in place of the vector comprising a promoter operably linked to an influenza virus M cDNA linked to a transcription termination sequence, e.g., a vector comprising a promoter operably linked to an influenza virus M1 cDNA linked to a transcription termination sequence and a vector comprising a promoter operably linked to an influenza virus M2 cDNA linked to a transcription termination sequence.

A plurality of the vectors of the invention may be physically linked or each vector may be present on an individual plasmid or other, e.g., linear, nucleic acid delivery vehicle. In one embodiment, each vRNA production vector is on a separate plasmid. In one embodiment, each mRNA production vector is on a separate plasmid.

The invention also provides a method to prepare influenza virus. The method comprises contacting a cell with a plurality of the vectors of the invention, e.g., sequentially or simultaneously, in an amount effective to yield infectious influenza virus. The invention also includes isolating virus from a cell contacted with the plurality of vectors. Thus, the invention further provides isolated virus, as well as a host cell contacted with the plurality of vectors or virus of the invention. In another embodiment, the invention includes contacting the cell with one or more vectors, either vRNA or protein production vectors, prior to other vectors, either vRNA or protein production vectors. In one embodiment, the promoter for vRNA vectors employed in the method is a RNA polymerase I promoter, a RNA polymerase II promoter, a RNA polymerase III promoter, a T3 promoter or a T7 promoter. In one embodiment, the RNA polymerase I promoter is a human RNA polymerase I promoter. In one embodiment, each vRNA vector employed in the method is on a separate plasmid. In one embodiment, the vRNA vectors employed in the method are on one plasmid or on two or three different plasmids. In one embodiment, each mRNA vector employed in the method is on a separate plasmid. In one embodiment, the mRNA vectors for PA, PB1, PB2 and NP employed in the method are on one plasmid or on two or three different plasmids.

In one embodiment, the invention provides a method to select for influenza viruses with enhanced replication in cell culture. The method includes providing cells suitable for influenza vaccine production; serially culturing one or more influenza virus isolates in the cells; and isolating serially cultured virus with enhanced growth relative to the one or more isolates prior to serial culture. In one embodiment, the cells are rodent or primate cells.

The methods of producing virus described herein, which do not require helper virus infection, are useful in viral mutagenesis studies, and in the production of vaccines (e.g., for AIDS, influenza, hepatitis B, hepatitis C, rhinovirus, filoviruses, malaria, herpes, and foot and mouth disease) and gene therapy vectors (e.g., for cancer, AIDS, adenosine deaminase, muscular dystrophy, ornithine transcarbamylase deficiency and central nervous system tumors). Thus, a virus for use in medical therapy (e.g., for a vaccine or gene therapy) is provided.

The invention also provides isolated viral polypeptides, and methods of preparing and using recombinant virus of the invention. The methods include administering to a host organism, e.g., a mammal, an effective amount of the influenza virus of the invention, e.g., an inactivated virus preparation, optionally in combination with an adjuvant and/or a carrier, e.g., in an amount effective to prevent or ameliorate infection of an animal such as a mammal by that virus or an antigenically closely related virus. In one embodiment, the virus is administered intramuscularly while in another embodiment, the virus is administered intranasally. In some dosing protocols, all doses may be administered intramuscularly or intranasally, while in others a combination of intramuscular and intranasal administration is employed. The vaccine may further contain other isolates of influenza virus including recombinant influenza virus, other pathogen(s), additional biological agents or microbial components, e.g., to form a multivalent vaccine. In one embodiment, intranasal vaccination, for instance containing with inactivated influenza virus, and a mucosal adjuvant may induce virus-specific IgA and neutralizing antibody in the nasopharynx as well as serum IgG.

The influenza virus of the invention may be employed with other anti-virals, e.g., amantadine, rimantadine, and/or neuraminidase inhibitors, e.g., may be administered separately in conjunction with those anti-virals, for instance, administered before, during and/or after.

### BRIEF DESCRIPTION OF THE FIGURES

FIGS. 1A-1E. Nucleotide sequence for PR8(Cambridge) genes (SEQ ID NOs:10-15).

FIG. 2: Overview of library passages and the identification of high-yield candidates.

FIG. 3. Number of clones with random mutations having specified HA titers.

FIG. 4. Titers of clones having selected mutations.

FIGS. 5A-5D. Growth curves of UW-PR8 viruses possessing previously identified mutations in PB2 (A), PB1 or PB1-F2 (B), PA (C), and NP, M or NS1 (D).

FIG. 6. Summary of mutations that confer high replicative property in MDCK cells.

FIGS. 7A-7B. A) Virus stocks were tested for HA titers (in 2ⁿ) and virus titers (in PFU/mL). B) Growth curves in MDCK cells.

FIGS. 8A-8C. A) HA titer of wild type (UW-PR8) and clone #4. B) Viral protein for wild type (UW-PR8) and #4. C) SDS-PAGE analysis of viral proteins of wild type and #4.

FIGS. 9A-9B. A) Comparison of titers of wild type virus (UW-PR8) and high replicative virus with mutations in M1. B) Growth kinetics of wild type virus (UW-PR8) and high replicative virus with mutations in M1.

FIGS. 10A-10M. A) Codon usage table for canines. B) Relative adaptiveness of wild type (UW-PR8) and “rare” codon optimized PB2 viruses. C) Relative adaptiveness of wild type (UW-PR8) and “all” codon optimized PB2 viruses. D) Growth kinetics of PB2 codon optimized viruses. E) Growth kinetics of viruses with codon optimized PB2, PB1, PA, or NP viral segment or combinations of segments. F-M) Sequence of PB2, PB1, PA and NP viral segments of UW-PR8 and sequence of canine codon-usage optimized PB2, PB1, PA and NP viral segments of UW-PR8 (SEQ ID no. 1-4 and 16-19).

FIGS. 11A-11C. A) Nucleotide position 4 of each gene of PR8 and Indo/NC/09. B) All 3′C4U mutant. C) Growth kinetics of a recombinant UW-PR8 virus encoding ‘C’ at position 4 of the PB2, PB1, and PA genes (black), and a mutant encoding ‘U’ at position 4 of all eight segments (red).

FIG. 12A-12C. Nucleotide and amino acid sequences for H7 and N9 (SEQ ID Nos. 20-27) which are exemplary sequences for use with the internal viral segment sequences disclosed herein useful to provide high titer influenza viruses for vaccines.

FIGS. 13A-13B. A) Schematic of chimeric HA and NA genes to increase virus titer. B) Growth kinetics of chimeric viruses.

FIGS. 14A-14B. A) Growth kinetics of viruses with combinations of mutations. B) PFU and HA titers of viruses with combinations of mutations.

FIG. 15. Screening in eggs.

FIG. 16. HA titers of 216 clones isolated from Vero cells.

FIG. 17. Recombinant viruses generated with different PR8 backbone mutations.

FIGS. 18A-18B. Overview of generation of viruses with enhanced growth in MDCK cells and Vero cells.

FIGS. 19A-19D. Exemplary high yield substitutions (relative to PR8 (UW)).

FIGS. 20A-20F. Growth kinetics and HA titers of reassortant viruses possessing one or several vRNAs of PR8-HY virus.

FIGS. 21A-21H. Viral polymerase activity in mini-replicon assays in 293T, MDCK, Vero, and DF1 cells. The PB2, PB1, PA, and NP proteins were derived from UW-PR8 wild-type (WT) virus or from the high-yield PR8-HY (HY) variant.

FIGS. 22A-22D. HA titer and virus yield for various recombinant viruses and parent reassortant virus in Vero cells.

FIGS. 23A-23B. Growth kinetics analysis of Hubei/1/10(H5N1) high yield candidates in serum-free (SF)-MDCK cells.

FIGS. 24A1-24B2. Growth kinetics analysis of HY candidate #9 backbone in SC-MDCK cells (A) and in eggs (B). HY candidate #9 was selected from SC-MDCK cells.

FIGS. 25A1-25C2. Growth kinetics analysis of HY candidate #5, #9, #16 backbone in eggs (A), in SC-MDCK cells (B) and in SF-MDCK cells (C).

### DETAILED DESCRIPTION

### Definitions

As used herein, the term “isolated” refers to in vitro preparation and/or isolation of a nucleic acid molecule, e.g., vector or plasmid, peptide or polypeptide (protein), or virus of the invention, so that it is not associated with in vivo substances, or is substantially purified from in vitro substances. An isolated virus preparation is generally obtained by in vitro culture and propagation, and/or via passage in eggs, and is substantially free from other infectious agents.

As used herein, “substantially purified” means the object species is the predominant species, e.g., on a molar basis it is more abundant than any other individual species in a composition, and preferably is at least about 80% of the species present, and optionally 90% or greater, e.g., 95%, 98%, 99% or more, of the species present in the composition.

As used herein, “substantially free” means below the level of detection for a particular infectious agent using standard detection methods for that agent.

A “recombinant” virus is one which has been manipulated in vitro, e.g., using recombinant DNA techniques, to introduce changes to the viral genome. Reassortant viruses can be prepared by recombinant or nonrecombinant techniques.

As used herein, the term “recombinant nucleic acid” or “recombinant DNA sequence or segment” refers to a nucleic acid, e.g., to DNA, that has been derived or isolated from a source, that may be subsequently chemically altered in vitro, so that its sequence is not naturally occurring, or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

As used herein, a “heterologous” influenza virus gene or viral segment is from an influenza virus source that is different than a majority of the other influenza viral genes or viral segments in a recombinant, e.g., reassortant, influenza virus.

The terms “isolated polypeptide”, “isolated peptide” or “isolated protein” include a polypeptide, peptide or protein encoded by cDNA or recombinant RNA including one of synthetic origin, or some combination thereof.

The term “recombinant protein” or “recombinant polypeptide” as used herein refers to a protein molecule expressed from a recombinant DNA molecule. In contrast, the term “native protein” is used herein to indicate a protein isolated from a naturally occurring (i.e., a nonrecombinant) source. Molecular biological techniques may be used to produce a recombinant form of a protein with identical properties as compared to the native form of the protein.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Alignments using these programs can be performed using the default parameters. Software for performing BLAST analyses is publicly available through the National Center for Biotechnology Information (http://www.ncbi.nlm.nih.gov/). The algorithm may involve first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm may also perform a statistical analysis of the similarity between two sequences. One measure of similarity provided by the BLAST algorithm may be the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

The BLASTN program (for nucleotide sequences) may use as defaults a wordlength (W) of 11, an expectation (E) of 10, a cutoff of 100, M=5, N=−4, and a comparison of both strands. For amino acid sequences, the BLASTP program may use as defaults a wordlength (W) of 3, an expectation (E) of 10, and the BLOSUM62 scoring matrix. See http://www.ncbi.nlm.nih.gov. Alignment may also be performed manually by inspection.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

### Influenza Virus Structure and Propagation

Influenza A viruses possess a genome of eight single-stranded negative-sense viral RNAs (vRNAs) that encode at least ten proteins. The influenza virus life cycle begins with binding of the hemagglutinin (HA) to sialic acid-containing receptors on the surface of the host cell, followed by receptor-mediated endocytosis. The low pH in late endosomes triggers a conformational shift in the HA, thereby exposing the N-terminus of the HA2 subunit (the so-called fusion peptide). The fusion peptide initiates the fusion of the viral and endosomal membrane, and the matrix protein (M1) and RNP complexes are released into the cytoplasm. RNPs consist of the nucleoprotein (NP), which encapsidates vRNA, and the viral polymerase complex, which is formed by the PA, PB1, and PB2 proteins. RNPs are transported into the nucleus, where transcription and replication take place. The RNA polymerase complex catalyzes three different reactions: synthesis of an mRNA with a 5′ cap and 3′ polyA structure, of a full-length complementary RNA (cRNA), and of genomic vRNA using the cRNA as a template. Newly synthesized vRNAs, NP, and polymerase proteins are then assembled into RNPs, exported from the nucleus, and transported to the plasma membrane, where budding of progeny virus particles occurs. The neuraminidase (NA) protein plays a crucial role late in infection by removing sialic acid from sialyloligosaccharides, thus releasing newly assembled virions from the cell surface and preventing the self aggregation of virus particles. Although virus assembly involves protein-protein and protein-vRNA interactions, the nature of these interactions is largely unknown.

Although influenza B and C viruses are structurally and functionally similar to influenza A virus, there are some differences. For example, influenza B virus has a viral segment with both NA and NB sequences. Influenza C virus has only seven viral segments.

### Cell Lines That Can Be Used in the Present Invention

Any cell, e.g., any avian or mammalian cell, such as a human, e.g., 293T or PER.C6® cells, or canine, e.g., MDCK, bovine, equine, feline, swine, ovine, rodent, for instance mink, e.g., MvLu1 cells, or hamster, e.g., CHO cells, or non-human primate, e.g., Vero cells, including mutant cells, which supports efficient replication of influenza virus can be employed to isolate and/or propagate influenza viruses. Isolated viruses can be used to prepare a reassortant virus. In one embodiment, host cells for vaccine production are continuous mammalian or avian cell lines or cell strains. A complete characterization of the cells to be used, may be conducted so that appropriate tests for purity of the final product can be included. Data that can be used for the characterization of a cell includes (a) information on its origin, derivation, and passage history; (b) information on its growth and morphological characteristics; (c) results of tests of adventitious agents; (d) distinguishing features, such as biochemical, immunological, and cytogenetic patterns which allow the cells to be clearly recognized among other cell lines; and (e) results of tests for tumorigenicity. In one embodiment, the passage level, or population doubling, of the host cell used is as low as possible.

In one embodiment, the cells are WHO certified, or certifiable, continuous cell lines. The requirements for certifying such cell lines include characterization with respect to at least one of genealogy, growth characteristics, immunological markers, virus susceptibility tumorigenicity and storage conditions, as well as by testing in animals, eggs, and cell culture. Such characterization is used to confirm that the cells are free from detectable adventitious agents. In some countries, karyology may also be required. In addition, tumorigenicity may be tested in cells that are at the same passage level as those used for vaccine production. The virus may be purified by a process that has been shown to give consistent results, before vaccine production (see, e.g., World Health Organization, 1982).

Virus produced by the host cell may be highly purified prior to vaccine or gene therapy formulation. Generally, the purification procedures result in extensive removal of cellular DNA and other cellular components, and adventitious agents. Procedures that extensively degrade or denature DNA may also be used.

### Influenza Vaccines

A vaccine of the invention includes an isolated recombinant influenza virus of the invention, and optionally one or more other isolated viruses including other isolated influenza viruses, one or more immunogenic proteins or glycoproteins of one or more isolated influenza viruses or one or more other pathogens, e.g., an immunogenic protein from one or more bacteria, non-influenza viruses, yeast or fungi, or isolated nucleic acid encoding one or more viral proteins (e.g., DNA vaccines) including one or more immunogenic proteins of the isolated influenza virus of the invention. In one embodiment, the influenza viruses of the invention may be vaccine vectors for influenza virus or other pathogens.

A complete virion vaccine may be concentrated by ultrafiltration and then purified by zonal centrifugation or by chromatography. Viruses other than the virus of the invention, such as those included in a multivalent vaccine, may be inactivated before or after purification using formalin or beta-propiolactone, for instance.

A subunit vaccine comprises purified glycoproteins. Such a vaccine may be prepared as follows: using viral suspensions fragmented by treatment with detergent, the surface antigens are purified, by ultracentrifugation for example. The subunit vaccines thus contain mainly HA protein, and also NA. The detergent used may be cationic detergent for example, such as hexadecyl trimethyl ammonium bromide (Bachmeyer, 1975), an anionic detergent such as ammonium deoxycholate (Laver & Webster, 1976); or a nonionic detergent such as that commercialized under the name TRITON X100. The hemagglutinin may also be isolated after treatment of the virions with a protease such as bromelin, and then purified. The subunit vaccine may be combined with an attenuated virus of the invention in a multivalent vaccine.

A split vaccine comprises virions which have been subjected to treatment with agents that dissolve lipids. A split vaccine can be prepared as follows: an aqueous suspension of the purified virus obtained as above, inactivated or not, is treated, under stirring, by lipid solvents such as ethyl ether or chloroform, associated with detergents. The dissolution of the viral envelope lipids results in fragmentation of the viral particles. The aqueous phase is recuperated containing the split vaccine, constituted mainly of hemagglutinin and neuraminidase with their original lipid environment removed, and the core or its degradation products. Then the residual infectious particles are inactivated if this has not already been done. The split vaccine may be combined with an attenuated virus of the invention in a multivalent vaccine.

Inactivated Vaccines. Inactivated influenza virus vaccines are provided by inactivating replicated virus using known methods, such as, but not limited to, formalin or β-propiolactone treatment. Inactivated vaccine types that can be used in the invention can include whole-virus (WV) vaccines or subvirion (SV) (split) vaccines. The WV vaccine contains intact, inactivated virus, while the SV vaccine contains purified virus disrupted with detergents that solubilize the lipid-containing viral envelope, followed by chemical inactivation of residual virus.

In addition, vaccines that can be used include those containing the isolated HA and NA surface proteins, which are referred to as surface antigen or subunit vaccines.

Live Attenuated Virus Vaccines. Live, attenuated influenza virus vaccines, such as those including a recombinant virus of the invention can be used for preventing or treating influenza virus infection. Attenuation may be achieved in a single step by transfer of attenuated genes from an attenuated donor virus to a replicated isolate or reassorted virus according to known methods. Since resistance to influenza A virus is mediated primarily by the development of an immune response to the HA and/or NA glycoproteins, the genes coding for these surface antigens come from the reassorted viruses or clinical isolates. The attenuated genes are derived from an attenuated parent. In this approach, genes that confer attenuation generally do not code for the HA and NA glycoproteins.

Viruses (donor influenza viruses) are available that are capable of reproducibly attenuating influenza viruses, e.g., a cold adapted (ca) donor virus can be used for attenuated vaccine production. Live, attenuated reassortant virus vaccines can be generated by mating the ca donor virus with a virulent replicated virus. Reassortant progeny are then selected at 25° C. (restrictive for replication of virulent virus), in the presence of an appropriate antiserum, which inhibits replication of the viruses bearing the surface antigens of the attenuated ca donor virus. Useful reassortants are: (a) infectious, (b) attenuated for seronegative non-adult mammals and immunologically primed adult mammals, (c) immunogenic and (d) genetically stable. The immunogenicity of the ca reassortants parallels their level of replication. Thus, the acquisition of the six transferable genes of the ca donor virus by new wild-type viruses has reproducibly attenuated these viruses for use in vaccinating susceptible mammals both adults and non-adult.

Other attenuating mutations can be introduced into influenza virus genes by site-directed mutagenesis to rescue infectious viruses bearing these mutant genes. Attenuating mutations can be introduced into non-coding regions of the genome, as well as into coding regions. Such attenuating mutations can also be introduced into genes other than the HA or NA, e.g., the PB2 polymerase gene. Thus, new donor viruses can also be generated bearing attenuating mutations introduced by site-directed mutagenesis, and such new donor viruses can be used in the production of live attenuated reassortants vaccine candidates in a manner analogous to that described above for the ca donor virus. Similarly, other known and suitable attenuated donor strains can be reassorted with influenza virus to obtain attenuated vaccines suitable for use in the vaccination of mammals.

In one embodiment, such attenuated viruses maintain the genes from the virus that encode antigenic determinants substantially similar to those of the original clinical isolates. This is because the purpose of the attenuated vaccine is to provide substantially the same antigenicity as the original clinical isolate of the virus, while at the same time lacking pathogenicity to the degree that the vaccine causes minimal chance of inducing a serious disease condition in the vaccinated mammal.

The viruses in a multivalent vaccine can thus be attenuated or inactivated, formulated and administered, according to known methods, as a vaccine to induce an immune response in an animal, e.g., a mammal. Methods are well-known in the art for determining whether such attenuated or inactivated vaccines have maintained similar antigenicity to that of the clinical isolate or high growth strain derived therefrom. Such known methods include the use of antisera or antibodies to eliminate viruses expressing antigenic determinants of the donor virus; chemical selection (e.g., amantadine or rimantidine); HA and NA activity and inhibition; and nucleic acid screening (such as probe hybridization or PCR) to confirm that donor genes encoding the antigenic determinants (e.g., HA or NA genes) are not present in the attenuated viruses.

### Pharmaceutical Compositions

Pharmaceutical compositions of the present invention, suitable for inoculation, e.g., nasal, parenteral or oral administration, comprise one or more influenza virus isolates, e.g., one or more attenuated or inactivated influenza viruses, a subunit thereof, isolated protein(s) thereof, and/or isolated nucleic acid encoding one or more proteins thereof, optionally further comprising sterile aqueous or non-aqueous solutions, suspensions, and emulsions. The compositions can further comprise auxiliary agents or excipients, as known in the art. The composition of the invention is generally presented in the form of individual doses (unit doses).

Conventional vaccines generally contain about 0.1 to 200 μg, e.g., 30 to 100 μg, of HA from each of the strains entering into their composition. The vaccine forming the main constituent of the vaccine composition of the invention may comprise a single influenza virus, or a combination of influenza viruses, for example, at least two or three influenza viruses, including one or more reassortant(s).

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

When a composition of the present invention is used for administration to an individual, it can further comprise salts, buffers, adjuvants, or other substances which are desirable for improving the efficacy of the composition. For vaccines, adjuvants, substances which can augment a specific immune response, can be used. Normally, the adjuvant and the composition are mixed prior to presentation to the immune system, or presented separately, but into the same site of the organism being immunized.

Heterogeneity in a vaccine may be provided by mixing replicated influenza viruses for at least two influenza virus strains, such as 2-20 strains or any range or value therein. Vaccines can be provided for variations in a single strain of an influenza virus, using techniques known in the art.

A pharmaceutical composition according to the present invention may further or additionally comprise at least one chemotherapeutic compound, for example, for gene therapy, immunosuppressants, anti-inflammatory agents or immune enhancers, and for vaccines, chemotherapeutics including, but not limited to, gamma globulin, amantadine, guanidine, hydroxybenzimidazole, interferon-α, interferon-β, interferon-γ, tumor necrosis factor-alpha, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir.

The composition can also contain variable but small quantities of endotoxin-free formaldehyde, and preservatives, which have been found safe and not contributing to undesirable effects in the organism to which the composition is administered.

### Pharmaceutical Purposes

The administration of the composition (or the antisera that it elicits) may be for either a “prophylactic” or “therapeutic” purpose. When provided prophylactically, the compositions of the invention which are vaccines are provided before any symptom or clinical sign of a pathogen infection becomes manifest. The prophylactic administration of the composition serves to prevent or attenuate any subsequent infection. When provided prophylactically, the gene therapy compositions of the invention, are provided before any symptom or clinical sign of a disease becomes manifest. The prophylactic administration of the composition serves to prevent or attenuate one or more symptoms or clinical signs associated with the disease.

When provided therapeutically, a viral vaccine is provided upon the detection of a symptom or clinical sign of actual infection. The therapeutic administration of the compound(s) serves to attenuate any actual infection. When provided therapeutically, a gene therapy composition is provided upon the detection of a symptom or clinical sign of the disease. The therapeutic administration of the compound(s) serves to attenuate a symptom or clinical sign of that disease.

Thus, a vaccine composition of the present invention may be provided either before the onset of infection (so as to prevent or attenuate an anticipated infection) or after the initiation of an actual infection. Similarly, for gene therapy, the composition may be provided before any symptom or clinical sign of a disorder or disease is manifested or after one or more symptoms are detected.

A composition is said to be “pharmacologically acceptable” if its administration can be tolerated by a recipient mammal. Such an agent is said to be administered in a “therapeutically effective amount” if the amount administered is physiologically significant. A composition of the present invention is physiologically significant if its presence results in a detectable change in the physiology of a recipient patient, e.g., enhances at least one primary or secondary humoral or cellular immune response against at least one strain of an infectious influenza virus.

The “protection” provided need not be absolute, i.e., the influenza infection need not be totally prevented or eradicated, if there is a statistically significant improvement compared with a control population or set of mammals. Protection may be limited to mitigating the severity or rapidity of onset of symptoms or clinical signs of the influenza virus infection.

### Pharmaceutical Administration

A composition of the present invention may confer resistance to one or more pathogens, e.g., one or more influenza virus strains, by either passive immunization or active immunization. In active immunization, an attenuated live vaccine composition is administered prophylactically to a host (e.g., a mammal), and the host's immune response to the administration protects against infection and/or disease. For passive immunization, the elicited antisera can be recovered and administered to a recipient suspected of having an infection caused by at least one influenza virus strain. A gene therapy composition of the present invention may yield prophylactic or therapeutic levels of the desired gene product by active immunization.

In one embodiment, the vaccine is provided to a mammalian female (at or prior to pregnancy or parturition), under conditions of time and amount sufficient to cause the production of an immune response which serves to protect both the female and the fetus or newborn (via passive incorporation of the antibodies across the placenta or in the mother's milk).

The present invention thus includes methods for preventing or attenuating a disorder or disease, e.g., an infection by at least one strain of pathogen. As used herein, a vaccine is said to prevent or attenuate a disease if its administration results either in the total or partial attenuation (i.e., suppression) of a clinical sign or condition of the disease, or in the total or partial immunity of the individual to the disease. As used herein, a gene therapy composition is said to prevent or attenuate a disease if its administration results either in the total or partial attenuation (i.e., suppression) of a clinical sign or condition of the disease, or in the total or partial immunity of the individual to the disease.

A composition having at least one influenza virus of the present invention, including one which is attenuated and one or more other isolated viruses, one or more isolated viral proteins thereof, one or more isolated nucleic acid molecules encoding one or more viral proteins thereof, or a combination thereof, may be administered by any means that achieve the intended purposes.

For example, administration of such a composition may be by various parenteral routes such as subcutaneous, intravenous, intradermal, intramuscular, intraperitoneal, intranasal, oral or transdermal routes. Parenteral administration can be accomplished by bolus injection or by gradual perfusion over time.

A typical regimen for preventing, suppressing, or treating an influenza virus related pathology, comprises administration of an effective amount of a vaccine composition as described herein, administered as a single treatment, or repeated as enhancing or booster dosages, over a period up to and including between one week and about 24 months, or any range or value therein.

According to the present invention, an “effective amount” of a composition is one that is sufficient to achieve a desired effect. It is understood that the effective dosage may be dependent upon the species, age, sex, health, and weight of the recipient, kind of concurrent treatment, if any, frequency of treatment, and the nature of the effect wanted. The ranges of effective doses provided below are not intended to limit the invention and represent dose ranges.

The dosage of a live, attenuated or killed virus vaccine for an animal such as a mammalian adult organism may be from about 10²-10¹⁵, e.g., 10³-10¹², plaque forming units (PFU)/kg, or any range or value therein. The dose of inactivated vaccine may range from about 0.1 to 1000, e.g., 30 to 100 μg, of HA protein. However, the dosage should be a safe and effective amount as determined by conventional methods, using existing vaccines as a starting point.

The dosage of immunoreactive HA in each dose of replicated virus vaccine may be standardized to contain a suitable amount, e.g., 30 to 100 μg or any range or value therein, or the amount recommended by government agencies or recognized professional organizations. The quantity of NA can also be standardized, however, this glycoprotein may be labile during purification and storage.

The dosage of immunoreactive HA in each dose of replicated virus vaccine can be standardized to contain a suitable amount, e.g., 1-50 μg or any range or value therein, or the amount recommended by the U.S. Public Health Service (PHS), which is usually 15 μg per component for older children (greater than or equal to 3 years of age), and 7.5 μg per component for children less than 3 years of age. The quantity of NA can also be standardized, however, this glycoprotein can be labile during the processor purification and storage (Kendal et al., 1980; Kerr et al., 1975). Each 0.5-ml dose of vaccine may contains approximately 1-50 billion virus particles, and preferably 10 billion particles.

### Exemplary Embodiments

In one embodiment, an isolated influenza virus having PA, PB1, PB2, NP, NS, M, NA and HA viral segments is provided, wherein the PB1 viral segment encodes a PB1 with a residue other than isoleucine at position 711 or the M viral segment encodes a M1 with a residue other than methionine at position 128, wherein the influenza virus has enhanced replication relative to a corresponding influenza virus having a PB1 viral segment that encodes a PB1 with an isoleucine at position 711 or having a M viral segment that encodes a M1 with methionine at position 128. In one embodiment, the NA viral segment is heterologous to the PA, PB1, PB2, NP, NS, and M viral segments. In one embodiment, the NA and HA viral segments are from the same viral isolate. In one embodiment, the PB1 viral segment encodes a PB1 with a residue other than isoleucine at position 711 and the M viral segment encodes a M1 with a residue other than methionine at position 128. In one embodiment, the PA viral segment encodes a PA with a residue other than phenylalanine at position 105, a residue other than lysine at position 142, a residue other than serine at position 149, a residue other than serine at position 225, a residue other than lysine at position 356, a residue other than threonine at position 357, a residue other than arginine at residue 401, or a residue other than isoleucine at position 550, or any combination thereof. In one embodiment, the PB1 viral segment further encodes a PB1 with a residue other than methionine at position 40, a residue other than glutamic acid at position 112, a residue other than glycine at position 180, or a residue other than glutamine at residue 247, or any combination thereof. In one embodiment, the PB2 viral segment encodes a PB2 a residue other than methionine at position 202, a residue other than phenylalanine at position 323, or a residue other than isoleucine at position 504, or any combination thereof. In one embodiment, the NP viral segment encodes a NP with a residue other than arginine at position 74, a residue other than isoleucine at position 116, or a residue other than asparagine at position 417, or any combination thereof. In one embodiment, the NS viral segment encodes a NS1 with a residue other than alanine at position 30, a residue other than lysine at position 55, or a residue other than arginine at position 118, or any combination thereof.

In one embodiment, the PA viral segment encodes a PA with a residue other than phenylalanine at position 105, a residue other than lysine at position 142, a residue other than serine at position 149, a residue other than serine at position 225, a residue other than arginine at position 256, a residue other than asparagine at position 350, a residue other than lysine at position 356, a residue other than threonine at position 357, a residue other than alanine at position 369, a residue other than arginine at residue 401, or a residue other than isoleucine at position 550, or any combination thereof.

In one embodiment, the PB1 viral segment encodes a PB1 with a residue other than methionine at position 40, a residue other than methionine at position 92, a residue other than asparagine at position 105, a residue other than glutamic acid at position 112, a residue other than glutamic acid at position 178, a residue other than glycine at position 180, a residue other than glutamine at residue 247, a residue other than methionine at position 290, a residue other than methionine at position 325, a residue other than isoleucine at position 711, or any combination thereof.

In one embodiment, the PB2 viral segment encodes a PB2 a residue other than arginine at position 62, a residue other than methionine at position 202, a residue other than alanine at position 221, a residue other than phenylalanine at position 323, a residue other than alanine at position 370, a residue other than methionine at position 467, a residue other than isoleucine at position 504, a residue other than tyrosine at position 704, or a residue other than lysine at position 721, or any combination thereof.

In one embodiment, the NP viral segment encodes a NP with a residue other than arginine at position 74, a residue other than lysine at position 103, a residue other than isoleucine at position 116, a residue other than valine at position 194, or a residue other than asparagine at position 417, or any combination thereof.

In one embodiment, the M viral segment encodes a M1 with a residue other than serine at position 126, with a residue other than methionine at position 128, a residue other than methionine at position 244,or a M2 with a residue other than tryptophan at position 15, or any combination thereof.

In one embodiment, the NS viral segment encodes a NS1 with a residue other than alanine at position 30, a residue other than lysine at position 55, a residue other than aspartic acid at position 101, or a residue other than arginine at position 118, or any combination thereof.

In one embodiment, the virus has a V, A, L, G, S, T, C, or M at position 711 in PB1, has an A, L, G, V, S, T, I, or C at position 40 in PB1, has an A, L, G, V, S, T, I, or C at position 112 in PB1, has an A, L, W, Y, F, V, S, T, I, or C at position 180 in PB1, has a H, R, K, D or E at position 247 in PB1, has a V, A, L, G, S, T, C, or I at position 290 in PB1, has a V, A, L, G, S, T, C, or I at position 92 in PB1, has a V, A, L, G, S, T, C, or I at position 325 in PB1, has a H, R, K, D, or E at position 105 in PB1, has a H, R, or K at position 178 in PB1, or any combination thereof.

In one embodiment, the virus has a V, A, L, or G at position 711 in PB1, has an A, L, G, V, or I at position 40 in PB1, has an A, L, G, V, S, T, or I at position 112 in PB1, has a W, Y, or F, at position 180 in PB1, has a H, R, K, D or E at position 247 in PB1, has a V, A, L, G, or I at position 290 in PB1, has a V, A, L, G, or I at position 92 in PB1, has a V, A, L, G, or I at position 325 in PB1, has a H, R, or K at position 105 in PB1, has a R or K at position 178 in PB1, or any combination thereof.

In one embodiment, the virus has a K, H, D, E, Q or N at position 74 in NP, has a R or H at position 103 in NP, has a I, A, L, or G at position 194 in NP, has a L, V, G, A, S, or T at position 116 in NP, has a D, E, Q, K, or H at position 417 in NP, or any combination thereof.

In one embodiment, the virus of has a P, W, F, S or T at position 30 in NS1, has E, D, Q, or N at position 55, has a L, I, V, G, A or T at position 101 in NS1, has a K, H, D, E, Q, or N at position 118 in NS1, or any combination thereof.

In one embodiment, the virus has a E, D, H, R or K at position 142 in PA, has a C, L, I, V, G, A, or T at position 225 in PA, has a L, I, V, S, G, A, or T at position 256 in PA, has a L, I, V, G, A, or T at position 350 in PA, has a L, I, V, G, S, or T at position 356 in PA, has a K, H, D, E, N or Q at position 401 in PA, or any combination thereof.

In one embodiment, the virus has a V, A, L, G, S, or T at position 504 in PB2, has a A, L, G, S, T, I, or C at position 202 in PB2, has a A, L, G, S, T, I, or C at position 323 in PB2, has a V, L, G, or I at position 221 in PB2, has a V, L, G, T or I at position 370 in PB2, has a V, L, G, S, T or I at position 62 in PB2, has a V, L, A, G, S, T, or lat position 467 in PB2, has a K, H, D, E, N or Q at position 704 in PB2, has a R, H, D, E, N or Q at position 721 in PB2, or any combination thereof.

In one embodiment, the virus has a V, A, L, G, or T at position 126 in M1, has a V, A, L, G, T, or S at position 128 in M1, has a R, K, D, E or H at position 244 in M1, has a A, L, G, S, T, or I at position 15 in M2, or any combination thereof.

In one embodiment, the virus has a V, A, L, G, S, T, C, or M at position 711 in PB1, has a A, L, G, V, S, T, I, or C at position 40 in PB1, has a A, L, W, Y, F, V, S, T, I, or C at position 180 in PB1; has a L, V, G, A, S, C or T at position 116 in NP; has a P, W, F, L, I, V, G, S, C or T at position 30 in NS1, has a K, H, D, E, Q, or N at position 118 in NS1; has a L, I, V, G, A, S, C or T at position 128 in M1; has a K, H, D, E, N or Q at position 401 in PA; has a V, A, L, G, S, T, C, or M at position 504 in PB2; or any combination thereof. In one embodiment, the virus has a V, A, L, G, S, T, C, or M at position 711 in PB1, has a H, R, K, D, E, D or E at position 247 in PB1; has a K, H, D, E, Q or N at position 74 in NP; has E, D, Q, or N at position 55 in NS1; has a L, I, V, G, A, S, C or T at position 128 in M1; has a C, L, I, V, G, A, or T at position 142 in PA; has a A, L, G, S, T, I, or C at position 202 in PB2, has a A, L, G, S, T, I, or C at position 323 in PB2; or any combination thereof. In one embodiment, the virus has a V, A, L, G, S, T, C, or M at position 711 in PB1, has a A, L, G, V, S, T, I, or C at position 112 in PB1; has a K, H, D, E, Q or N at position 74 in NP, has a D, E, Q, K, or H at position 417 in NP; has E, D, Q, or N at position 55; has a L, I, V, G, A, S, C or T at position 128 in M1; has a C, L, I, V, G, A, or T at position 225 in PA; has a V, A, L, G, S, T, C, or M at position 504 in PB2; or any combination thereof. In one embodiment, the virus has 142N, 225C, 356R, or 550L in PA; has one or more of 112G, 247H, 507V, or 644A in PB1; has one or more of 202L, 323L or 504V in PB2; has one or more of 74K, 112L, 116L, 417D, or 442A in NP; 97A and/or 100H in M1; and/or 55E and/or 140Q in NS1, or any combination thereof. In one embodiment, the virus has at least one of 202L and/or 323L in PB2, 247H in PB1 or 74K in NP and optionally at least one of 142N in PA1, 55K in NS1 or 97A and/or 100H in M1.

In one embodiment, the virus has at least one of 202L and/or 323L in PB2, 247H in PB1 or 74K in NP and at least one of 142N in PA1, 55K in NS1 or 97A and/or 100H in M1. In one embodiment, the virus has 202L and/or 323L in PB2. In one embodiment, the virus has 247H in PB1. In one embodiment, the virus has 55E in NS1. In one embodiment, the virus has 142C in PA. In one embodiment, the virus has 74K in NP. In one embodiment, the virus has 504V in PB2. In one embodiment, the virus has 74K, 116L or 417D in NP. In one embodiment, the virus has 30P, 55E or 118K in NS1. In one embodiment, the virus has 225C or 401K in PA. In one embodiment, the virus has a V, A, L, G, S, T, C, or M at position 711 in PB1. In one embodiment, the virus has a L, I, V, G, A, S, C or T at position 128 in M1. In one embodiment, the virus has a U at position 4 in the viral segment for any one of PB1, PB2 or PA. In one embodiment, the PA, PB1, PB2, NP, NS, and M viral segments comprise sequences for at least one of the following: a PB1 having the amino acid sequence encoded by SEQ ID NO:2 or PB1 with at least 95% amino acid sequence identity to the PB1 encoded by SEQ ID NO:2; a PB2 having the amino acid sequence encoded by SEQ ID NO:3 or PB2 with at least 95% amino acid sequence identity to the PB2 encoded by SEQ ID NO:3; a PA having the amino acid sequence encoded by SEQ ID NO:1 or PA with at least 95% amino acid sequence identity to the PA encoded by SEQ ID NO:1; a NP having the amino acid sequence encoded by SEQ ID NO:4 or NP with at least 95% amino acid sequence identity to the NP encoded by SEQ ID NO:4; a M1 and/or M2 having the amino acid sequence encoded by SEQ ID NO:5 or M1 and/or M2 with at least 95% amino acid sequence identity to the M1 and/or M2 encoded by SEQ ID NO:5; or a NS1 and/or NS2 having the amino acid sequence encoded by SEQ ID NO:6 or NS1 and/or NS2 with at least 95% amino acid sequence identity to the NS1 and/or NS2 encoded by SEQ ID NO:6 or wherein the PA, PB1, PB2, NP, NS, and M viral segments comprise sequences for at least one of the following: a PB1 having the amino acid sequence encoded by SEQ ID NO:10 or PB1 with at least 95% amino acid sequence identity to the PB1 encoded by SEQ ID NO:10; a PB2 having the amino acid sequence encoded by SEQ ID NO:11 or PB2 with at least 95% amino acid sequence identity to the PB2 encoded by SEQ ID NO:11; a PA having the amino acid sequence encoded by SEQ ID NO:12 or PA with at least 95% amino acid sequence identity to the PA encoded by SEQ ID NO:12; a NP having the amino acid sequence encoded by SEQ ID NO:13 or NP with at least 95% amino acid sequence identity to the NP encoded by SEQ ID NO:13; a M1 and/or M2 having the amino acid sequence encoded by SEQ ID NO:14 or M1 and/or M2 with at least 95% amino acid sequence identity to the M1 and/or M2 encoded by SEQ ID NO:14; or a NS1 and/or NS2 having the amino acid sequence encoded by SEQ ID NO:15 or NS1 and/or NS2 with at least 95% amino acid sequence identity to the NS1 and/or NS2 encoded by SEQ ID NO:15.

A vaccine having the isolated virus is also provided.

In one embodiment, a plurality of influenza virus vectors for preparing a reassortant virus is provided. In one embodiment, the plurality includes but is not limited to a vector for vRNA production comprising a promoter operably linked to an influenza virus PA DNA linked to a transcription termination sequence, a vector for vRNA production comprising a promoter operably linked to an influenza virus PB1 DNA linked to a transcription termination sequence, a vector for vRNA production comprising a promoter operably linked to an influenza virus PB2 DNA linked to a transcription termination sequence, a vector for vRNA production comprising a promoter operably linked to an influenza virus HA DNA linked to a transcription termination sequence, a vector for vRNA production comprising a promoter operably linked to an influenza virus NP DNA linked to a transcription termination sequence, a vector for vRNA production comprising a promoter operably linked to an influenza virus NA DNA linked to a transcription termination sequence, a vector for vRNA production comprising a promoter operably linked to an influenza virus M DNA linked to a transcription termination sequence, and a vector for vRNA production comprising a promoter operably linked to an influenza virus NS cDNA linked to a transcription termination sequence, wherein the PB1, PB2, PA, NP, NS, and M DNAs in the vectors for vRNA production are from one or more influenza vaccine virus isolates, wherein the NA DNA in the vector for vRNA production of NA has sequences for a heterologous NA, and wherein the HA DNA in the vector for vRNA production of HA has sequences for a heterologous HA, wherein the PB1 DNA encodes a PB1 with a residue other than isoleucine at position 711 or the M DNA encodes a M1 with a residue other than methionine at position 128; and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2. In one embodiment, the PB1, PB2, PA, NP, NS, and M DNAs in the vectors for vRNA production have a sequence corresponding to one that encodes a polypeptide having at least 95% amino acid sequence identity to a corresponding polypeptide encoded by SEQ ID NOs:1-6 or 10-15. In one embodiment, the promoter for vRNA vectors is a RNA polymerase I promoter, a RNA polymerase II promoter, a RNA polymerase III promoter, a T3 promoter or a T7 promoter. In one embodiment, the HA is H1, H3, H5, H7 or H9. In one embodiment, the PA, PB1, and PB2 viral segments, and optionally NP, NS, and M viral segments has a promoter C to a mutation.

Further provided is a method to prepare influenza virus, comprising: contacting a cell with: a vector for vRNA production comprising a promoter operably linked to an influenza virus PA DNA linked to a transcription termination sequence, a vector for vRNA production comprising a promoter operably linked to an influenza virus PB1 DNA linked to a transcription termination sequence, a vector for vRNA production comprising a promoter operably linked to an influenza virus PB2 DNA linked to a transcription termination sequence, a vector for vRNA production comprising a promoter operably linked to an influenza virus HA DNA linked to a transcription termination sequence, a vector for vRNA production comprising a promoter operably linked to an influenza virus NP DNA linked to a transcription termination sequence, a vector for vRNA production comprising a promoter operably linked to an influenza virus NA DNA linked to a transcription termination sequence, a vector for vRNA production comprising a promoter operably linked to an influenza virus M DNA linked to a transcription termination sequence, and a vector for vRNA production comprising a promoter operably linked to an influenza virus NS DNA linked to a transcription termination sequence, wherein the PB1, PB2, PA, NP, NS, and M DNAs in the vectors for vRNA production are from one or more influenza vaccine virus isolates, wherein the NA DNA in the vector for vRNA production of NA has sequences for a heterologous NA, and wherein the HA DNA in the vector for vRNA production of HA has sequences for a heterologous HA, wherein the PB1 DNA encodes a PB1 with a residue other than isoleucine at position 711 or the M DNA encodes a M1 with a residue other than methionine at position 128; and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2; in an amount effective to yield infectious influenza virus. In one embodiment, the cell is an avian or a mammalian cell. In one embodiment, the cell is a Vero cell, a human cell or a MDCK cell. In one embodiment, the PB1, PB2, PA, NP, NS, and M DNAs in the vectors for vRNA productions have a sequence that corresponds to one that encodes a polypeptide having at least 95% amino acid sequence identity to a corresponding polypeptide encoded by SEQ ID NOs:1-6 or 10-15. In one embodiment, the virus is isolated. In one embodiment, the PB1 viral segment encodes a PB1 with a residue other than isoleucine at position 711 and the M viral segment encodes a M1 with a residue other than methionine at position 128. In one embodiment, the PA viral segment encodes a PA with a residue other than phenylalanine at position 105, a residue other than lysine at position 142, a residue other than serine at position 149, a residue other than serine at position 225, a residue other than lysine at position 356, a residue other than threonine at position 357, a residue other than arginine at residue 401, or a residue other than isoleucine at position 550, or any combination thereof; or wherein the PB1 viral segment further encodes a PB1 with a residue other than methionine at position 40, a residue other than glutamic acid at position 112, a residue other than glycine at position 180, or a residue other than glutamine at residue 247, or any combination thereof; or wherein the PB2 viral segment encodes a PB2 a residue other than methionine at position 202, a residue other than phenylalanine at position 323, or a residue other than isoleucine at position 504, or any combination thereof or wherein the NP viral segment encodes a NP with a residue other than arginine at position 74, a residue other than isoleucine at position 116, or a residue other than asparagine at position 417, or any combination thereof; or wherein the NS viral segment encodes a NS1 with a residue other than alanine at position 30, a residue other than lysine at position 55, or a residue other than arginine at position 118, or any combination thereof. In one embodiment, the virus has a V, A, L, G, S, T, C, or M at position 711 in PB1, has a A, L, G, V, S, T, I, or C at position 40 in PB1, has a A, L, G, V, S, T, I, or C at position 112 in PB1, has a A, L, W, Y, F, V, S, T, I, or C at position 180 in PB1, has a H, R, K, D, E, D o E at position 247 in PB1, or any combination thereof; or has a K, H, D, E, Q or N at position 74 in NP, has a L, V, G, A, S, C or T at position 116 in NP, has a D, E, Q, K, or H at position 417 in NP, or any combination thereof; or has a P, W, F, L, I, V, G, S, C or T at position 30 in NS1, has E, D, Q, or N at position 55, has a K, H, D, E, Q, or N at position 118 in NS1, has a L, I, V, G, A, S, C or T at position 128 in M1, or any combination thereof; or has a C, L, I, V, G, A, or T at position 142 in PA, has a C, L, I, V, G, A, or T at position 225 in PA, has a K, H, D, E, N or Q at position 401 in PA, or any combination thereof; or has an V, A, L, G, S, T, C, or M at position 504 in PB2, has a A, L, G, S, T, I, or C at position 202 in PB2, has a A, L, G, S, T, I, or C at position 323 in PB2, or any combination thereof. In one embodiment, the virus has a V, A, L, G, S, T, C, or M at position 711 in PB1, has a A, L, G, V, S, T, I, or C at position 40 in PB1, has a A, L, W, Y, F, V, S, T, I, or C at position 180 in PB1; has a L, V, G, A, S, C or T at position 116 in NP; has a P, W, F, L, I, V, G, S, C or T at position 30 in NS1, has a K, H, D, E, Q, or N at position 118 in NS1; has a L, I, V, G, A, S, C or T a position 128 in M1; has a K, H, D, E, N or Q at position 401 in PA; has an V, A, L, G, S, T, C, or M at position 504 in PB2; or any combination thereof. In one embodiment, the virus has a V, A, L, G, S, T, C, or M at position 711 in PB1, has a H, R, K, D, E, D or E at position 247 in PB1; has a K, H, D, E, Q or N at position 74 in NP; has E, D, Q, or N at position 55 in NS1; has a L, I, V, G, A, S, C or T at position 128 in M1; has a C, L, I, V, G, A, or T at position 142 in PA; has a A, L, G, S, T, I, or C at position 202 in PB2, has a A, L, G, S, T, I, or C at position 323 in PB2; or any combination thereof. In one embodiment, the virus has a V, A, L, G, S, T, C, or M at position 711 in PB1, has a A, L, G, V, S, T, I, or C at position 112 in PB1; has a K, H, D, E, Q or N at position 74 in NP, has a D, E, Q, K, or H at position 417 in NP; has E, D, Q, or N at position 55; has a L, I, V, G, A, S, C or T at position 128 in M1; has a C, L, I, V, G, A, or T at position 225 in PA; has an V, A, L, G, S, T, C, or M at position 504 in PB2; or any combination thereof. In one embodiment, the virus has one or more of 142N, 225C, 356R, or 550L in PA; has one or more of 112G, 247H, 507V, or 644A in PB1; has one or more of 202L, 323L or 504V in PB2; has one or more of 74K, 112L, 116L, 417D, or 442A in NP; 97A and/or 100H in M1; and/or 55E and/or 140Q in NS1, or any combination thereof. In one embodiment, the virus has at least one of 202L and/or 323L in PB2, 247H in PB1 or 74K in NP and optionally at least one of 142N in PA1, 55K in NS1 or 97A and/or 100H in M1. In one embodiment, the virus has at least one of 202L and/or 323L in PB2, 247H in PB1 or 74K in NP and at least one of 142N in PA1, 55K in NS1 or 97A and/or 100H in M1. In one embodiment, the virus has 202L and/or 323L in PB2. In one embodiment, the virus has 247H in PB1. In one embodiment, the virus has 55E in NS1. In one embodiment, the virus has 142C in PA. In one embodiment, the virus has 74K in NP. In one embodiment, the virus has 504V in PB2. In one embodiment, the virus has 74K, 116L or 417D in NP. In one embodiment, the virus has 30P, 55E or 118K in NS1. In one embodiment, the virus has 225C or 401K in PA. In one embodiment, the virus has a V, A, L, G, S, T, C, or M at position 711 in PB1. In one embodiment, the virus has a L, I, V, G, A, S, C or T at position 128 in M1. In one embodiment, at least one of PA, PB1, or PB2 viral segments has a C to U promoter mutation.

In other embodiments, the virus has a residue at position 30 or 31 in PA other than the residue encoded by SEQ ID NO:1 or 10; a residue in PB1 other than R54, T59, G62, A63, E75, D76, E78, P79, S80, R327, M507, L624, V644, I667, N694, L695, E697, F699, F700, P701, S702, Y705, S713, and/or M714; a residue in PB2 other than I57, T58, A59, K61, M66, R368, E391, Q591, E677, D678, and/or P679, in PB2; a residue in NP other than I112, N224, R293, M371, R422 or T442; a residue in M1 other than P90, V97, and/or Y100; or a residue in NS1 other than T49, R140, S161 and/or A223. In one embodiment, the PB1 viral segment and/or the NS viral segment encodes a polypeptide having a residue other than glycine, serine, serine, glutamine or asparagine at position 62, 261, 361, 621, and/or 654 in PB1 or a residue other than arginine at position 81 in F2.

In one embodiment, the isolated recombinant influenza virus has PA, PB1, PB2, NP, NS, and M viral segments from a first influenza vaccine virus isolate, a heterologous, recombinant or chimeric influenza virus NA viral segment, and a heterologous, recombinant or chimeric HA viral segment, wherein one, two or more of the PA, PB1, PB2, NP, NS, and M viral segments have selected amino acid residues at positions 30, 31, 105, 142, 149, 225, 356, 357, 401, and/or 550 in PA; positions 40, 54, 59, 62, e.g., 62A, 63, 75, 76, 78, 79, 80, 112, 180, 247, 261, e.g., 161G, 327, 361, e.g., 361R, 507, 621, e.g., 621R, 624, 644, 654, e.g., 654S, 667, 694, 695, 697, 699, 700, 701, 702, 705, 713, and/or 714 in PB1; positions 57, 58, 59, 61, 66, 202, 323, 368, 391, 504, 591, 677, 678, and/or 679, in PB2; positions 74, 112, 116, 224, 293, 371, 377, 417, 422 or 442 in NP; positions 90, 97 and/or 100 in M1; or positions 30, 49, 55, 118, e.g., 118K, 140, 161, and/or 223 in NS1, and optionally an HA with a residue other than glutamic acid, lysine, glutamine, leucine, valine, phenylalanine, lysine or methionine at position 136, 162, 179, 182, 184, 252, 449, or 476, respectively, e.g., a HA segment with one or more of 136D, 162E, 179L, 182V, 184I, 252I, 449E or 476I, or optionally a NA with a residue other than leucine or alanine at residue 55 or 265, respectively, e.g., 55S or 265V. In one embodiment, the isolated virus has 142N, 225C, 356R, or 550L in PA; has one or more of 112G, 247H, 507V, or 644A in PB1; has one or more of 202L, 323L or 504V in PB2; has one or more of 74K, 112L, 116L, 417D, or 442A in NP; 97A and/or 100H in M1; and/or 55E and/or 140Q in NS1, or combinations thereof, e.g., has at least one of 202L and/or 323L in PB2, 247H in PB1 or 74K in NP and optionally at least one of 142N in PA1, 55K in NS1 or 97A and/or 100H in M1 or has at least one of 202L and/or 323L in PB2, 247H in PB1 or 74K in NP and at least one of 142N in PA1, 55K in NS1 or 97A and/or 100H in M1. In one embodiment, the virus has at least one of 202L and/or 323L in PB2, 247H in PB1 or 74K in NP and optionally at least one of 142N in PA1, 55K in NS1 or 97A and/or 100H in M1. In one embodiment, the virus has at least one of 202L and/or 323L in PB2, 247H in PB1 or 74K in NP and at least one of 142N in PA1, 55K in NS1 or 97A and/or 100H in M1. In one embodiment, the isolated virus has 202L and/or 323L in PB2, and optionally has 247H in PB1 and optionally 74K in NP. In one embodiment, the isolated virus has 247H in PB1 and optionally 74K in NP. In one embodiment, the isolated virus has 40I, 40L, 112G, 180W, 247H, 507V, or 644A in PB1 and optionally has 202L and/or 323L in PB2, and optionally has 74K, 112L, 116L, 377N, 417D, or 422L in NP, and optionally has 30P, 118K, 161T or 140Q in NS1, and optionally has 142N, 225C, 356R, 401K, or 550L in PA. In one embodiment, the isolated virus has 40I, 40L, 112G, 180W, 247H, 507V, or 644A in PB1. In one embodiment, the isolated virus has 202L and/or 323L in PB2. In one embodiment, the isolated virus has 74K, 112L, 116L, 377N, 417D, or 422L in NP. In one embodiment, the isolated virus has 30P, 118K, 161T or 140Q in NS1. In one embodiment, the isolated virus has142N, 225C, 356R, 401K, or 550L in PA. In one embodiment, the selected amino acid residues at specified positions in the PA is/are at position(s) 97, 105, 142, 149, 225, 356, 357, 401, 404, and/or 421. In one embodiment, the selected amino acid residues at specified positions in the PB1 is/are at position(s) 12, 40, 54, 59, 62, 63, 66, 75, 76, 78, 79, 80, 180, 247, 507, 624, 644, 694, 695, 697, 699, 700, 701, 705, 713, 714, and/or 762. In one embodiment, the selected amino acid residues at specified positions in the PB2 is/are at position(s) 57, 58, 59, 61, 66, 202, 243, 323, 504, 677, 678, and/or 679. In one embodiment, the selected amino acid residues at specified positions in the NP is/are at position(s) 74, 112, 116, 224, 293, 417, and/or 442. In one embodiment, the selected amino acid residues at specified positions in the M1 is/are at position(s) 90, 97, and/or 100. In one embodiment, the selected amino acid residues at specified positions in the NS1 is/are at position(s) 49, 30, 55, 161, and/or 223. In one embodiment, the selected amino acid residues at specified positions in the PA is/are at position(s) 97, 105, 142, 149, 225, 356, 357, 401, 404, and/or 421; and optionally the selected amino acid residues at specified positions in the PB1 is/are at position(s) 12, 40, 54, 59, 62, 63, 66, 75, 76, 78, 79, 80, 180, 247, 507, 624, 644, 694, 695, 697, 699, 700, 701, 705, 713, 714, and/or 762, in any combination with the selected residues for PA; and optionally the selected amino acid residues at specified positions in the PB2 is/are at position(s) 57, 58, 59, 61, 66, 202, 243, 323, 504, 677, 678, and/or 679 in any combination with the selected residues for PA and/or PB1; and optionally the selected amino acid residues at specified positions in the NP is/are at position(s) 74, 112, 116, 224, 293, 417, and/or 442 any combination with the selected residues for PA, PB1 and/or PB2; and optionally the selected amino acid residues at specified positions in the M1 is/are at position(s) 90, 97, and/or 100 any combination with the selected residues for PA, PB1, PB2, and/or NP; and optionally the selected amino acid residues at specified positions in the NS1 is/are at position(s) 49, 30, 55, 161, and/or 223, or in any combination with the selected residues for PA, PB1, PB2, NP, and/or M1.

For any of the exemplary viruses disclosed above, in one embodiment, the PA, PB1, PB2, NP, NS, and M viral segments comprise sequences for at least one of the following: a PB1 having the amino acid sequence encoded by SEQ ID NO:2 or PB1 with at least 95% amino acid sequence identity to the PB1 encoded by SEQ ID NO:2; a PB2 having the amino acid sequence encoded by SEQ ID NO:3 or PB2 with at least 95% amino acid sequence identity to the PB2 encoded by SEQ ID NO:3; a PA having the amino acid sequence encoded by SEQ ID NO:1 or PA with at least 95% amino acid sequence identity to the PA encoded by SEQ ID NO:1; a NP having the amino acid sequence encoded by SEQ ID NO:4 or NP with at least 95% amino acid sequence identity to the NP encoded by SEQ ID NO:4; a M1 and/or M2 having the amino acid sequence encoded by SEQ ID NO:5 or M1 and/or M2 with at least 95% amino acid sequence identity to the M1 and/or M2 encoded by SEQ ID NO:5; or a NS1 and/or NS2 having the amino acid sequence encoded by SEQ ID NO:6 or NS1 and/or NS2 with at least 95% amino acid sequence identity to the NS1 and/or NS2 encoded by SEQ ID NO:6, or the PA, PB1, PB2, NP, NS, and M viral segments comprise sequences for at least one of the following: a PB1 having the amino acid sequence encoded by SEQ ID NO:10 or PB1 with at least 95% amino acid sequence identity to the PB1 encoded by SEQ ID NO:10; a PB2 having the amino acid sequence encoded by SEQ ID NO:11 or PB2 with at least 95% amino acid sequence identity to the PB2 encoded by SEQ ID NO:11; a PA having the amino acid sequence encoded by SEQ ID NO:12 or PA with at least 95% amino acid sequence identity to the PA encoded by SEQ ID NO:12; a NP having the amino acid sequence encoded by SEQ ID NO:13 or NP with at least 95% amino acid sequence identity to the NP encoded by SEQ ID NO:13; a M1 and/or M2 having the amino acid sequence encoded by SEQ ID NO:14 or M1 and/or M2 with at least 95% amino acid sequence identity to the M1 and/or M2 encoded by SEQ ID NO:14; or a NS1 and/or NS2 having the amino acid sequence encoded by SEQ ID NO:15 or NS1 and/or NS2 with at least 95% amino acid sequence identity to the NS1 and/or NS2 encoded by SEQ ID NO:15.

For any of the exemplary viruses disclosed above, in one embodiment, at least one of the PA, PB1, PB2, NP, NS, and M viral segments has a C to U promoter mutation.

Any of the isolated viruses disclosed herein may be employed in a vaccine.

In one embodiment, the invention provides a plurality of influenza virus vectors for preparing a reassortant. In one embodiment, the plurality includes a vector for vRNA production comprising a promoter operably linked to an influenza virus PA DNA linked to a transcription termination sequence, a vector for vRNA production comprising a promoter operably linked to an influenza virus PB1 DNA linked to a transcription termination sequence, a vector for vRNA production comprising a promoter operably linked to an influenza virus PB2 DNA linked to a transcription termination sequence, a vector for vRNA production comprising a promoter operably linked to an influenza virus HA DNA linked to a transcription termination sequence, a vector for vRNA production comprising a promoter operably linked to an influenza virus NP DNA linked to a transcription termination sequence, a vector for vRNA production comprising a promoter operably linked to an influenza virus NA DNA linked to a transcription termination sequence, a vector for vRNA production comprising a promoter operably linked to an influenza virus M DNA linked to a transcription termination sequence, and a vector for vRNA production comprising a promoter operably linked to an influenza virus NS cDNA linked to a transcription termination sequence, wherein the PB1, PB2, PA, NP, NS, and M DNAs in the vectors for vRNA production are from one or more influenza vaccine virus isolates, wherein the NA DNA in the vector for vRNA production of NA has sequences for a heterologous NA, and wherein the HA DNA in the vector for vRNA production of HA has sequences for a heterologous HA, 30, 31, 105, 142, 149, 225, 356, 357, 401, and/or 550 in PA; 40, 54, 59, 62, 63, 75, 76, 78, 79, 80, 112, 180, 247, 327, 507, 624, 644, 667, 694, 695, 697, 699, 700, 701, 702, 705, 713, or 714 and/or 247 in PB1; 57, 58, 59, 61, 66, 202, 323, 368, 391, 504, 591, 677, 678, or 679, 202 and/or 323 in PB2; 74, 112, 116, 224, 293, 371, 377, 417, 422 and/or 442 in NP; 90, 97 and/or 100 in M1; or 30, 49, 55, 118,140, 161 and/or 223 in NS; and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2. In one embodiment, the PB1, PB2, PA, NP, NS, and M DNAs in the vectors for vRNA production have a sequence corresponding to one that encodes a polypeptide having at least 95% amino acid sequence identity to a corresponding polypeptide encoded by SEQ ID NOs:1-6 or 10-15. In one embodiment, the promoter for vRNA vectors is a RNA polymerase I promoter, a RNA polymerase II promoter, a RNA polymerase III promoter, a T3 promoter or a T7 promoter. In one embodiment, the NA is N9. In one embodiment, the HA is H7. In one embodiment, the PA, PB1, PB2, NP, NS, and/or M viral segments has/have a promoter C to a mutation.

In one embodiment, the invention provides a method to prepare influenza virus. The method includes contacting a cell with: a vector for vRNA production comprising a promoter operably linked to an influenza virus PA DNA linked to a transcription termination sequence, a vector for vRNA production comprising a promoter operably linked to an influenza virus PB1 DNA linked to a transcription termination sequence, a vector for vRNA production comprising a promoter operably linked to an influenza virus PB2 DNA linked to a transcription termination sequence, a vector for vRNA production comprising a promoter operably linked to an influenza virus HA DNA linked to a transcription termination sequence, a vector for vRNA production comprising a promoter operably linked to an influenza virus NP DNA linked to a transcription termination sequence, a vector for vRNA production comprising a promoter operably linked to an influenza virus NA DNA linked to a transcription termination sequence, a vector for vRNA production comprising a promoter operably linked to an influenza virus M DNA linked to a transcription termination sequence, and a vector for vRNA production comprising a promoter operably linked to an influenza virus NS DNA linked to a transcription termination sequence, wherein the PB1, PB2, PA, NP, NS, and M DNAs in the vectors for vRNA production are from one or more influenza vaccine virus isolates, wherein the NA DNA in the vector for vRNA production of NA has sequences for a heterologous NA, and wherein the HA DNA in the vector for vRNA production of HA has sequences for a heterologous HA, 30, 31, 105, 142, 149, 225, 356, 357, 401, and/or 550 in PA; 40, 54, 59, 62, 63, 75, 76, 78, 79, 80, 112, 180, 247, 327, 507, 624, 644, 667, 694, 695, 697, 699, 700, 701, 702, 705, 713, and/or 714 and/or 247 in PB1; 57, 58, 59, 61, 66, 202, 323, 368, 391, 504, 591, 677, 678, and/or 679, 202 and/or 323 in PB2; 74, 112, 116, 224, 293, 371, 377, 417, 422 and/or 442 in NP; 90, 97 and/or 100 in M1; or 30, 49, 55, 118, 140, 161 or 223 in NS; and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2; in an amount effective to yield infectious influenza virus. In one embodiment, the cell is an avian cell or a mammalian cell, e.g., a Vero cell, a human cell or a MDCK cell. In one embodiment, the PB1, PB2, PA, NP, NS, and M DNAs in the vectors for vRNA productions have a sequence that corresponds to one that encodes a polypeptide having at least 95% amino acid sequence identity to a corresponding polypeptide encoded by SEQ ID NOs:1-6 or 10-15. In one embodiment, the method includes isolating the virus. In one embodiment, at least one of PA, PB1, or PB2 viral segments has a C to U promoter mutation.

Further provided is a vector for vRNA or mRNA expression of influenza virus PA having at least 95% amino acid sequence identity to a polypeptide encoded by SEQ ID NO:1 and having a threonine at position 30, a lysine at position 31, cysteine at position 105 or a lysine at position 401; a vector for vRNA or mRNA expression of influenza virus PB1 having at least 95% amino acid sequence identity to a polypeptide encoded by SEQ ID NO:2 and having a leucine at position 40, an alanine or isoleucine at position 54, glycine at position 112, histidine at position 247, valine at position 507, alanine at position 644, or cysteine at position 713; a vector for vRNA or mRNA expression of PB2 having at least 95% amino acid sequence identity to a polypeptide encoded by SEQ ID NO:3 and a leucine at position 202 and/or 323; a vector for vRNA or mRNA expression of influenza virus NP having at least 95% amino acid sequence identity to a polypeptide encoded by SEQ ID NO:4 and having a lysine at position 74, leucine at position 116, isoleucine at position 224, lysine at position 293, asparagine at position 377, or aspartic acid at position 417; a vector for vRNA or mRNA expression of influenza virus NS1 having at least 95% amino acid sequence identity to a NS1 polypeptide encoded by SEQ ID NO:6 and having a proline at position 30, alanine at position 49, lysine at position 118, glutamine at position 140, threonine at position 161, or glutamic acid at position 223; and a vector for vRNA or mRNA expression of influenza virus M1 having at least 95% amino acid sequence identity to a M1 polypeptide encoded by SEQ ID NO:5 and having a serine at position 90.

The invention will be described by the following nonlimiting examples.

### EXAMPLE 1

### Methods

### Cells and Viruses

293T human embryonic kidney cells are maintained in Dulbecco's modified Eagle's minimal essential medium (DMEM) with 10% fetal calf serum and antibiotics. Madin-Darby canine kidney (MDCK) cells are grown in MEM with 5% newborn calf serum and antibiotics. African green monkey Vero WCB cells, which had been established after biosafety tests for use in human vaccine production (Sugawara et al., 2002), are maintained in serum-free VP-SFM medium (GIBCO-BRL) with antibiotics. Cells are maintained at 37° C. in 5% CO₂.

### Construction of Plasmids and Reverse Genetics

To generate reassortants of influenza A viruses, a plasmid-based reverse genetics (Neumann et al., 1999) is used. The full-length cDNAs were cloned into a plasmid under control of the human polymerase I promoter and the mouse RNA polymerase I terminator (Poll plasmids).

A previously produced series of Poll constructs, derived from A/WSN/33 (H1N1; WSN) or PR8 strains is used, for reverse genetics (Horimoto et al., 2006; Neumann et al., 1999). The World Health Organization (WHO) recommends A/Puerto Rico/8/34 (H1N1; PR8) as a donor virus, because of its safety in humans (Wood & Robertson, 2004; Webby & Webster, 2003).

Plasmids expressing WSN or PR8 NP, PA, PB1, or PB2 under control of the chicken actin, e.g., beta-actin, promoter are used for all reverse genetics experiments (Horimoto et al., 2006; Neumann et al., 1999). Briefly, Poll plasmids and protein expression plasmids are mixed with a transfection reagent, Trans-IT 293T (Panvera), incubated at room temperature for 15 minutes, and then added to 293T cells. Transfected cells are incubated in Opti-MEM I (GIBCO-BRL) for 48 hours. For reverse genetics in Vero WCB cells, an electroporator (Amaxa) is used to transfect the plasmid mixtures according to the manufacturer's instructions. Sixteen hours after transfection, freshly prepared Vero WCB cells were added onto the transfected cells and TPCK-trypsin (1 μg/mL) is added to the culture 6 hours later. Transfected cells are incubated in serum-free VP-SFM for a total of 4 days. Supernatants containing infectious viruses are harvested, and may be biologically cloned by limiting dilution.

A recombinant virus having the HA and NA genes from A/Hong Kong/213/2003 (H5N1) and the remainder of the type A influenza virus genes from PR8(UW) was prepared. The titer of the recombinant virus was 10^{10.67} EID₅₀/mL, and the HA titer was 1:1600

TABLE 1

| Virus possessing | | | | | | | |
|---|---|---|---|---|---|---|---|
| PR8 genes together | | | | | | | |
| with the following | HA titer (HAU/mL) in each dilition | | | | | | |
| HA and NA genes | 10-2 | 10-3 | 10-4 | 10-5 | 10-6 | 10-7 | 10-8 |
| WSN-HA NA | 160 | 40 | 40 | 320 | 40 | 640 | <1 |
| HK-HAavir NA | 400 | 800 | 400 | 400 | 400 | 800 | <1 |

The sequences of PR8 (UW) genes are as follows:

PA
(SEQ ID NO: 1)
AGCGAAAGCA GGTACTGATC CAAAATGGAA GATTTTGTGC GACAATGCTT CAATCCGATG
ATTGTCGAGC TTGCGGAAAA AACAATGAAA GAGTATGGGG AGGACCTGAA AATCGAAACA
AACAAATTTG CAGCAATATG CACTCACTTG GAAGTATGCT TCATGTATTC AGATTTTCAC
TTCATCAATG AGCAAGGCGA GTCAATAATC GTAGAACTTG GTGATCCAAA TGCACTTTTG
AAGCACAGAT TTGAAATAAT CGAGGGAAGA GATCGCACAA TGGCCTGGAC AGTAGTAAAC
AGTATTTGCA ACACTACAGG GGCTGAGAAA CCAAAGTTTC TACCAGATTT GTATGATTAC
AAGGAGAATA GATTCATCGA AATTGGAGTA ACAAGGAGAG AAGTTCACAT ATACTATCTG
GAAAAGGCCA ATAAAATTAA ATCTGAGAAA ACACACATCC ACATTTTCTC GTTCACTGGG
GAAGAAATGG CCACAAAGGC AGACTACACT CTCGATGAAG AAAGCAGGGC TAGGATCAAA
ACCAGACTAT TCACCATAAG ACAAGAAATG GCCAGCAGAG GCCTCTGGGA TTCCTTTCGT
CAGTCCGAGA GAGGAGAAGA GACAATTGAA GAAAGGTTTG AAATCACAGG AACAATGCGC
AAGCTTGCCG ACCAAAGTCT CCCGCCGAAC TTCTCCAGCC TTGAAAATTT TAGAGCCTAT
GTGGATGGAT TCGAACCGAA CGGCTACATT GAGGGCAAGC TGTCTCAAAT GTCCAAAGAA
GTAAATGCTA GAATTGAACC TTTTTTGAAA ACAACACCAC GACCACTTAG ACTTCCGAAT
GGGCCTCCCT GTTCTCAGCG GTCCAAATTC CTGCTGATGG ATGCCTTAAA ATTAAGCATT
GAGGACCCAA GTCATGAAGG AGAGGGAATA CCGCTATATG ATGCAATCAA ATGCATGAGA
ACATTCTTTG GATGGAAGGA ACCCAATGTT GTTAAACCAC ACGAAAAGGG AATAAATCCA
AATTATCTTC TGTCATGGAA GCAAGTACTG GCAGAACTGC AGGACATTGA GAATGAGGAG
AAAATTCCAA AGACTAAAAA TATGAAGAAA ACAAGTCAGC TAAAGTGGGC ACTTGGTGAG
AACATGGCAC CAGAAAAGGT AGACTTTGAC GACTGTAAAG ATGTAGGTGA TTTGAAGCAA
TATGATAGTG ATGAACCAGA ATTGAGGTCG CTTGCAAGTT GGATTCAGAA TGAGTTTAAC
AAGGCATGCG AACTGACAGA TTCAAGCTGG ATAGAGCTCG ATGAGATTGG AGAAGATGTG
GCTCCAATTG AACACATTGC AAGCATGAGA AGGAATTATT TCACATCAGA GGTGTCTCAC
TGCAGAGCCA CAGAATACAT AATGAAGGGA GTGTACATCA ATACTGCCTT GCTTAATGCA
TCTTGTGCAG CAATGGATGA TTTCCAATTA ATTCCAATGA TAAGCAAGTG TAGAACTAAG
GAGGGAAGGC GAAAGACCAA CTTGTATGGT TTCATCATAA AAGGAAGATC CCACTTAAGG
AATGACACCG ACGTGGTAAA CTTTGTGAGC ATGGAGTTTT CTCTCACTGA CCCAAGACTT
GAACCACATA AATGGGAGAA GTACTGTGTT CTTGAGATAG GAGATATGCT TATAAGAAGT
GCCATAGGCC AGGTTTCAAG GCCCATGTTC TTGTATGTGA GAACAAATGG AACCTCAAAA
ATTAAAATGA AATGGGGAAT GGAGATGAGG CGTTGCCTCC TCCAGTCACT TCAACAAATT
GAGAGTATGA TTGAAGCTGA GTCCTCTGTC AAAGAGAAAG ACATGACCAA AGAGTTCTTT
GAGAACAAAT CAGAAACATG GCCCATTGGA GAGTCCCCCA AAGGAGTGGA GGAAAGTTCC
ATTGGGAAGG TCTGCAGGAC TTTATTAGCA AAGTCGGTAT TCAACAGCTT GTATGCATCT
CCACAACTAG AAGGATTTTC AGCTGAATCA AGAAAACTGC TTCTTATCGT TCAGGCTCTT
AGGGACAACC TGGAACCTGG GACCTTTGAT CTTGGGGGGC TATATGAAGC AATTGAGGAG
TGCCTGATTA ATGATCCCTG GGTTTTGCTT AATGCTTCTT GGTTCAACTC CTTCCTTACA
CATGCATTGA GTTAGTTGTG GCAGTGCTAC TATTTGCTAT CCATACTGTC CAAAAAAGTA
CCTTGTTTCT ACT
PB1
(SEQ ID NO: 2)
AGCGAAAGCAGGCAAACCATTTGAATGGATGTCAATCCGACCTTACTTTTCTTAAAAGTGCCAGCACAAA
ATGCTATAAGCACAACTTTCCCTTATACTGGAGACCCTCCTTACAGCCATGGGACAGGAACAGGATACAC
CATGGATACTGTCAACAGGACACATCAGTACTCAGAAAAGGGAAGATGGACAACAAACACCGAAACTGG
AGCACCGCAACTCAACCCGATTGATGGGCCACTGCCAGAAGACAATGAACCAAGTGGTTATGCCCAAAC
AGATTGTGTATTGGAGGCGATGGCTTTCCTTGAGGAATCCCATCCTGGTATTTTTGAAAACTCGTGTATT
GAAACGATGGAGGTTGTTCAGCAAACACGAGTAGACAAGCTGACACAAGGCCGACAGACCTATGACTGG
ACTCTAAATAGAAACCAACCTGCTGCAACAGCATTGGCCAACACAATAGAAGTGTTCAGATCAAATGGCC
TCACGGCCAATGAGTCTGGAAGGCTCATAGACTTCCTTAAGGATGTAATGGAGTCAATGAACAAAGAAGA
AATGGGGATCACAACTCATTTTCAGAGAAAGAGACGGGTGAGAGACAATATGACTAAGAAAATGATAACA
CAGAGAACAATGGGTAAAAAGAAGCAGAGATTGAACAAAAGGAGTTATCTAATTAGAGCATTGACCCTGA
ACACAATGACCAAAGATGCTGAGAGAGGGAAGCTAAAACGGAGAGCAATTGCAACCCCAGGGATGCAAA
TAAGGGGGTTTGTATACTTTGTTGAGACACTGGCAAGGAGTATATGTGAGAAACTTGAACAATCAGGGTT
GCCAGTTGGAGGCAATGAGAAGAAAGCAAAGTTGGCAAATGTTGTAAGGAAGATGATGACCAATTCTCA
GGACACCGAACTTTCTTTCACCATCACTGGAGATAACACCAAATGGAACGAAAATCAGAATCCTCGGATG
TTTTTGGCCATGATCACATATATGACCAGAAATCAGCCCGAATGGTTCAGAAATGTTCTAAGTATTGCTCC
AATAATGTTCTCAAACAAAATGGCGAGACTGGGAAAAGGGTATATGTTTGAGAGCAAGAGTATGAAACTT
AGAACTCAAATACCTGCAGAAATGCTAGCAAGCATCGATTTGAAATATTTCAATGATTCAACAAGAAAGAA
GATTGAAAAAATCCGACCGCTCTTAATAGAGGGGACTGCATCATTGAGCCCTGGAATGATGATGGGCAT
GTTCAATATGTTAAGCACTGTATTAGGCGTCTCCATCCTGAATCTTGGACAAAAGAGATACACCAAGACTA
CTTACTGGTGGGATGGTCTTCAATCCTCTGACGATTTTGCTCTGATTGTGAATGCACCCAATCATGAAGG
GATTCAAGCCGGAGTCGACAGGTTTTATCGAACCTGTAAGCTACTTGGAATCAATATGAGCAAGAAAAAG
TCTTACATAAACAGAACAGGTACATTTGAATTCACAAGTTTTTTCTATCGTTATGGGTTTGTTGCCAATTTC
AGCATGGAGCTTCCCAGTTTTGGGGTGTCTGGGATCAACGAGTCAGCGGACATGAGTATTGGAGTTACT
GTCATCAAAAACAATATGATAAACAATGATCTTGGTCCAGCAACAGCTCAAATGGCCCTTCAGTTGTTCAT
CAAAGATTACAGGTACACGTACCGATGCCATATAGGTGACACACAAATACAAACCCGAAGATCATTTGAA
ATAAAGAAACTGTGGGAGCAAACCCGTTCCAAAGCTGGACTGCTGGTCTCCGACGGAGGCCCAAATTTA
TACAACATTAGAAATCTCCACATTCCTGAAGTCTGCCTAAAATGGGAATTGATGGATGAGGATTACCAGG
GGCGTTTATGCAACCCACTGAACCCATTTGTCAGCCATAAAGAAATTGAATCAATGAACAATGCAGTGAT
GATGCCAGCACATGGTCCAGCCAAAAACATGGAGTATGATGCTGTTGCAACAACACACTCCTGGATCCC
CAAAAGAAATCGATCCATCTTGAATACAAGTCAAAGAGGAGTACTTGAGGATGAACAAATGTACCAAAGG
TGCTGCAATTTATTTGAAAAATTCTTCCCCAGCAGTTCATACAGAAGACCAGTCGGGATATCCAGTATGGT
GGAGGCTATGGTTTCCAGAGCCCGAATTGATGCACGGATTGATTTCGAATCTGGAAGGATAAAGAAAGA
AGAGTTCACTGAGATCATGAAGATCTGTTCCACCATTGAAGAGCTCAGACGGCAAAAATAGTGAATTTAG
CTTGTCCTTCATGAAAAAATGCCTTGTTTCTACT
PB2
(SEQ ID NO: 3)
AGCGAAAGCA GGTCAATTAT ATTCAATATG GAAAGAATAA AAGAACTACG AAATCTAATG
TCGCAGTCTC GCACCCGCGA GATACTCACA AAAACCACCG TGGACCATAT GGCCATAATC
AAGAAGTACA CATCAGGAAG ACAGGAGAAG AACCCAGCAC TTAGGATGAA ATGGATGATG
GCAATGAAAT ATCCAATTAC AGCAGACAAG AGGATAACGG AAATGATTCC TGAGAGAAAT
GAGCAAGGAC AAACTTTATG GAGTAAAATG AATGATGCCG GATCAGACCG AGTGATGGTA
TCACCTCTGG CTGTGACATG GTGGAATAGG AATGGACCAA TAACAAATAC AGTTCATTAT
CCAAAAATCT ACAAAACTTA TTTTGAAAGA GTCGAAAGGC TAAAGCATGG AACCTTTGGC
CCTGTCCATT TTAGAAACCA AGTCAAAATA CGTCGGAGAG TTGACATAAA TCCTGGTCAT
GCAGATCTCA GTGCCAAGGA GGCACAGGAT GTAATGATGG AAGTTGTTTT CCCTAACGAA
GTGGGAGCCA GGATACTAAC ATCGGAATCG CAACTAACGA TAACCAAAGA GAAGAAAGAA
GAACTCCAGG ATTGCAAAAT TTCTCCTTTG ATGGTTGCAT ACATGTTGGA GAGAGAACTG
GTCCGCAAAA CGAGATTCCT CCCAGTGGCT GGTGGAACAA GCAGTGTGTA CATTGAAGTG
TTGCATTTGA CTCAAGGAAC ATGCTGGGAA CAGATGTATA CTCCAGGAGG GGAAGTGAGG
AATGATGATG TTGATCAAAG CTTGATTATT GCTGCTAGGA ACATAGTGAG AAGAGCTGCA
GTATCAGCAG ATCCACTAGC ATCTTTATTG GAGATGTGCC ACAGCACACA GATTGGTGGA
ATTAGGATGG TAGACATCCT TAGGCAGAAC CCAACAGAAG AGCAAGCCGT GGATATATGC
AAGGCTGCAA TGGGACTGAG AATTAGCTCA TCCTTCAGTT TTGGTGGATT CACATTTAAG
AGAACAAGCG GATCATCAGT CAAGAGAGAG GAAGAGGTGC TTACGGGCAA TCTTCAAACA
TTGAAGATAA GAGTGCATGA GGGATATGAA GAGTTCACAA TGGTTGGGAG AAGAGCAACA
GCCATACTCA GAAAAGCAAC CAGGAGATTG ATTCAGOTGA TAGTGAGTGG GAGAGACGAA
CAGTCGATTG CCGAAGCAAT AATTGTGGCC ATGGTATTTT CACAAGAGGA TTGTATGATA
AAAGCAGTCA GAGGTGATCT GAATTTCGTC AATAGGGCGA ATCAACGATT GAATCCTATG
CATCAACTTT TAAGACATTT TCAGAAGGAT GCGAAAGTGC TTTTTCAAAA TTGGGGAGTT
GAACCTATCG ACAATGTGAT GGGAATGATT GGGATATTGC CCGACATGAC TCCAAGCATC
GAGATGTCAA TGAGAGGAGT GAGAATCAGC AAAATGGGTG TAGATGAGTA CTCCAGCACG
GAGAGGGTAG TGGTGAGCAT TGACCGTTTT TTGAGAATCC GGGACCAACG AGGAAATGTA
CTACTGTCTC CCGAGGAGGT CAGTGAAACA CAGGGAACAG AGAAACTGAC AATAACTTAC
TCATCGTCAA TGATGTGGGA GATTAATGGT CCTGAATCAG TGTTGGTCAA TACCTATCAA
TGGATCATCA GAAACTGGGA AACTGTTAAA ATTCAGTGGT CCCAGAACCC TACAATGCTA
TACAATAAAA TGGAATTTGA ACCATTTCAG TCTTTAGTAC CTAAGGCCAT TAGAGGCCAA
TACAGTGGGT TTGTAAGAAC TCTGTTCCAA CAAATGAGGG ATGTGCTTGG GACATTTGAT
ACCGCACAGA TAATAAAACT TCTTCCCTTC GCAGCCGCTC CACCAAAGCA AAGTAGAATG
CAGTTCTCCT CATTTACTGT GAATGTGAGG GGATCAGGAA TGAGAATACT TGTAAGGGGC
AATTCTCCTG TATTCAACTA TAACAAGGCC ACGAAGAGAC TCACAGTTCT CGGAAAGGAT
GCTGGCACTT TAACTGAAGA CCCAGATGAA GGCACAGCTG GAGTGGAGTC CGCTGTTCTG
AGGGGATTCC TCATTCTGGG CAAAGAAGAC AAGAGATATG GGCCAGCACT AAGCATCAAT
GAACTGAGCA ACCTTGCGAA AGGAGAGAAG GCTAATGTGC TAATTGGGCA AGGAGACGTG
GTGTTGGTAA TGAAACGGAA ACGGGACTCT AGCATACTTA CTGACAGCCA GACAGCGACC
AAAAGAATTC GGATGGCCAT CAATTAGTGT CGAATAGTTT AAAAACGACC TTGTTTCTAC T
NP
(SEQ ID NO: 4)
AGCAAAAGCA GGGTAGATAA TCACTCACTG AGTGACATCA AAATCATGGC GTCTCAAGGC
ACCAAACGAT CTTACGAACA GATGGAGACT GATGGAGAAC GCCAGAATGC CACTGAAATC
AGAGCATCCG TCGGAAAAAT GATTGGTGGA ATTGGACGAT TCTACATCCA AATGTGCACC
GAACTCAAAC TCAGTGATTA TGAGGGACGG TTGATCCAAA ACAGCTTAAC AATAGAGAGA
ATGGTGCTCT CTGCTTTTGA CGAAAGGAGA AATAAATACC TTGAAGAACA TCCCAGTGCG
GGGAAAGATC CTAAGAAAAC TGGAGGACCT ATATACAGGA GAGTAAACGG AAAGTGGATG
AGAGAACTCA TCCTTTATGA CAAAGAAGAA ATAAGGCGAA TCTGGCGCCA AGCTAATAAT
GGTGACGATG CAACGGCTGG TCTGACTCAC ATGATGATCT GGCATTCCAA TTTGAATGAT
GCAACTTATC AGAGGACAAG AGCTCTTGTT CGCACCGGAA TGGATCCCAG GATGTGCTCT
CTGATGCAAG GTTCAACTCT CCCTAGGAGG TCTGGAGCCG CAGGTGCTGC AGTCAAAGGA
GTTGGAACAA TGGTGATGGA ATTGGTCAGA ATGATCAAAC GTGGGATCAA TGATCGGAAC
TTCTGGAGGG GTGAGAATGG ACGAAAAACA AGAATTGCTT ATGAAAGAAT GTGCAACATT
CTCAAAGGGA AATTTCAAAC TGCTGCACAA AAAGCAATGA TGGATCAAGT GAGAGAGAGC
CGGAACCCAG GGAATGCTGA GTTCGAAGAT CTCACTTTTC TAGCACGGTC TGCACTCATA
TTGAGAGGGT CGGTTGCTCA CAAGTCCTGC CTGCCTGCCT GTGTGTATGG ACCTGCCGTA
GCCAGTGGGT ACGACTTTGA AAGGGAGGGA TACTCTCTAG TCGGAATAGA CCCTTTCAGA
CTGCTTCAAA ACAGCCAAGT GTACAGCCTA ATCAGACCAA ATGAGAATCC AGCACACAAG
AGTCAACTGG TGTGGATGGC ATGCCATTCT GCCGCATTTG AAGATCTAAG AGTATTAAGC
TTCATCAAAG GGACGAAGGT GCTCCCAAGA GGGAAGCTTT CCACTAGAGG AGTTCAAATT
GCTTCCAATG AAAATATGGA GACTATGGAA TCAAGTACAC TTGAACTGAG AAGCAGGTAC
TGGGCCATAA GGACCAGAAG TGGAGGAAAC ACCAATCAAC AGAGGGCATC TGCGGGCCAA
ATCAGCATAC AACCTACGTT CTCAGTACAG AGAAATCTCC CTTTTGACAG AACAACCATT
ATGGCAGCAT TCAATGGGAA TACAGAGGGG AGAACATCTG ACATGAGGAC CGAAATCATA
AGGATGATGG AAAGTGCAAG ACCAGAAGAT GTGTCTTTCC AGGGGCGGGG AGTCTTCGAG
CTCTCGGACG AAAAGGCAGC GAGCCCGATC GTGCCTTCCT TTGACATGAG TAATGAAGGA
TCTTATTTCT TCGGAGACAA TGCAGAGGAG TACGACAATT AAAGAAAAAT ACCCTTGTTT CTACT
M
(SEQ ID NO: 5)
AGCAAAAGCA GGTAGATATT GAAAGATGAG TCTTCTAACC GAGGTCGAAA CGTACGTACT
CTCTATCATC CCGTCAGGCC CCCTCAAAGC CGAGATCGCA CAGAGACTTG AAGATGTCTT
TGCAGGGAAG AACACCGATC TTGAGGTTCT CATGGAATGG CTAAAGACAA GACCAATCCT
GTCACCTCTG ACTAAGGGGA TTTTAGGATT TGTGTTCACG CTCACCGTGC CCAGTGAGCG
AGGACTGCAG CGTAGACGCT TTGTCCAAAA TGCCCTTAAT GGGAACGGGG ATCCAAATAA
CATGGACAAA GCAGTTAAAC TGTATAGGAA GCTCAAGAGG GAGATAACAT TCCATGGGGC
CAAAGAAATC TCACTCAGTT ATTCTGCTGG TGCACTTGCC AGTTGTATGG GCCTCATATA
CAACAGGATG GGGGCTGTGA CCACTGAAGT GGCATTTGGC CTGGTATGTG CAACCTGTGA
ACAGATTGCT GACTCCCAGC ATCGGTCTCA TAGGCAAATG GTGACAACAA CCAATCCACT
AATCAGACAT GAGAACAGAA TGGTTTTAGC CAGCACTACA GCTAAGGCTA TGGAGCAAAT
GGCTGGATCG AGTGAGCAAG CAGCAGAGGC CATGGAGGTT GCTAGTCAGG CTAGACAAAT
GGTGCAAGCG ATGAGAACCA TTGGGACTCA TCCTAGCTCC AGTGCTGGTC TGAAAAATGA
TCTTCTTGAA AATTTGCAGG CCTATCAGAA ACGAATGGGG GTGCAGATGC AACGGTTCAA
GTGATCCTCT CACTATTGCC GCAAATATCA TTGGGATCTT GCACTTGACA TTGTGGATTC
TTGATCGTCT TTTTTTCAAA TGCATTTACC GTCGCTTTAA ATACGGACTG AAAGGAGGGC
CTTCTACGGA AGGAGTGCCA AAGTCTATGA GGGAAGAATA TCGAAAGGAA CAGCAGAGTG
CTGTGGATGC TGACGATGGT CATTTTGTCA GCATAGAGCT GGAGTAAAAA ACTACCTTGT TTCTACT
NS
(SEQ ID NO: 6)
AGCAAAAGCA GGGTGACAAA AACATAATGG ATCCAAACAC TGTGTCAAGC TTTCAGGTAG
ATTGCTTTCT TTGGCATGTC CGCAAACGAG TTGCAGACCA AGAACTAGGC GATGCCCCAT
TCCTTGATCG GCTTCGCCGA GATCAGAAAT CCCTAAGAGG AAGGGGCAGT ACTCTCGGTC
TGGACATCAA GACAGCCACA CGTGCTGGAA AGCAGATAGT GGAGCGGATT CTGAAAGAAG
AATCCGATGA GGCACTTAAA ATGACCATGG CCTCTGTACC TGCGTCGCGT TACCTAACTG
ACATGACTCT TGAGGAAATG TCAAGGGACT GGTCCATGCT CATACCCAAG CAGAAAGTGG
CAGGCCCTCT TTGTATCAGA ATGGACCAGG CGATCATGGA TAAGAACATC ATACTGAAAG
CGAACTTCAG TGTGATTTTT GACCGGCTGG AGACTCTAAT ATTGCTAAGG GCTTTCACCG
AAGAGGGAGC AATTGTTGGC GAAATTTCAC CATTGCCTTC TCTTCCAGGA CATACTGCTG
AGGATGTCAA AAATGCAGTT GGAGTCCTCA TCGGAGGACT TGAATGGAAT GATAACACAG
TTCGAGTCTC TGAAACTCTA CAGAGATTCG CTTGGAGAAG CAGTAATGAG AATGGGAGAC
CTCCACTCAC TCCAAAACAG AAACGAGAAA TGGCGGGAAC AATTAGGTCA GAAGTTTGAA
GAAATAAGAT GGTTGATTGA AGAAGTGAGA CACAAACTGA AGATAACAGA GAATAGTTTT
GAGCAAATAA CATTTATGCA AGCCTTACAT CTATTGCTTG AAGTGGAGCA AGAGATAAGA
ACTTTCTCGT TTCAGCTTAT TTAGTACTAA AAAACACCCT TGTTTCTACT
HA
(SEQ ID NO: 7)
AGCAAAAGCAGGGGAAAATAAAAACAACCAAAATGAAGGCAAACCTACTGGTCCTGTTATGTGCACTTGC
AGCTGCAGATGCAGACACAATATGTATAGGCTACCATGCGAACAATTCAACCGACACTGTTGACACAGTA
CTCGAGAAGAATGTGACAGTGACACACTCTGTTAACCTGCTCGAAGACAGCCACAACGGAAAACTATGTA
GATTAAAAGGAATAGCCCCACTACAATTGGGGAAATGTAACATCGCCGGATGGCTCTTGGGAAACCCAG
AATGCGACCCACTGCTTCCAGTGAGATCATGGTCCTACATTGTAGAAACACCAAACTCTGAGAATGGAAT
ATGTTATCCAGGAGATTTCATCGACTATGAGGAGCTGAGGGAGCAATTGAGCTCAGTGTCATCATTCGAA
AGATTCGAAATATTTCCCAAAGAAAGCTCATGGCCCAACCACAACACAAACGGAGTAACGGCAGCATGCT
CCCATGAGGGGAAAAGCAGTTTTTACAGAAATTTGCTATGGCTGACGGAGAAGGAGGGCTCATACCCAA
AGCTGAAAAATTCTTATGTGAACAAAAAAGGGAAAGAAGTCCTTGTACTGTGGGGTATTCATCACCCGCC
TAACAGTAAGGAACAACAGAATCTCTATCAGAATGAAAATGCTTATGTCTCTGTAGTGACTTCAAATTATA
ACAGGAGATTTACCCCGGAAATAGCAGAAAGACCCAAAGTAAGAGATCAAGCTGGGAGGATGAACTATT
ACTGGACCTTGCTAAAACCCGGAGACACAATAATATTTGAGGCAAATGGAAATCTAATAGCACCAATGTA
TGCTTTCGCACTGAGTAGAGGCTTTGGGTCCGGCATCATCACCTCAAACGCATCAATGCATGAGTGTAAC
ACGAAGTGTCAAACACCCCTGGGAGCTATAAACAGCAGTCTCCCTTACCAGAATATACACCCAGTCACAA
TAGGAGAGTGCCCAAAATACGTCAGGAGTGCCAAATTGAGGATGGTTACAGGACTAAGGAACATTCCGT
CCATTCAATCCAGAGGTCTATTTGGAGCCATTGCCGGTTTTATTGAAGGGGGATGGACTGGAATGATAGA
TGGATGGTATGGTTATCATCATCAGAATGAACAGGGATCAGGCTATGCAGCGGATCAAAAAAGCACACAA
AATGCCATTAACGGGATTACAAACAAGGTGAACACTGTTATCGAGAAAATGAACATTCAATTCACAGCTGT
GGGTAAAGAATTCAACAAATTAGAAAAAAGGATGGAAAATTTAAATAAAAAAGTTGATGATGGATTTCTGG
ACATTTGGACATATAATGCAGAATTGTTAGTTCTACTGGAAAATGAAAGGACTCTGGATTTCCATGACTCA
AATGTGAAGAATCTGTATGAGAAAGTAAAAAGCCAATTAAAGAATAATGCCAAAGAAATCGGAAATGGAT
GTTTGAGTTCTACCACAAGTGTGACAATGAATGCATGGAAAGTGTAAGAAATGGGACTTATGATTATCC
CAAATATTCAGAAGAGTCAAAGTTGAACAGGGAAAAGGTAGATGGAGTGAAATTGGAATCAATGGGGATC
TATCAGATTCTGGCGATCTACTCAACTGTCGCCAGTTCACTGGTGCTTTTGGTCTCCCTGGGGGCAATCA
GTTTCTGGATGTGTTCTAATGGATCTTTGCAGTGCAGAATATGCATCTGAGATTAGAATTTCAGAGATATG
AGGAAAAACACCCTTGTTTCTACT
NA
(SEQ ID NO: 8)
AGCAAAAGCAGGGGTTTAAAATGAATCCAAATCAGAAAATAATAACCATTGGATCAATCTGTCTGGTAGTC
GGACTAATTAGCCTAATATTGCAAATAGGGAATATAATCTCAATATGGATTAGCCATTCAATTCAAACTGG
AAGTCAAAACCATACTGGAATATGCAACCAAAACATCATTACCTATAAAAATAGCACCTGGGTAAAGGACA
CAACTTCAGTGATATTAACCGGCAATTCATCTCTTTGTCCCATCCGTGGGTGGGCTATATACAGCAAAGA
CAATAGCATAAGAATTGGTTCCAAAGGAGACGTTTTTGTCATAAGAGAGCCCTTTATTTCATGTTCTCACT
TGGAATGCAGGACCTTTTTTCTGACCCAAGGTGCCTTACTGAATGACAAGCATTCAAGTGGGACTGTTAA
GGACAGAAGCCCTTATAGGGCCTTAATGAGCTGCCCTGTCGGTGAAGCTCCGTCCCCGTACAATTCAAG
ATTTGAATCGGTTGCTTGGTCAGCAAGTGCATGTCATGATGGCATGGGCTGGCTAACAATCGGAATTTCA
GGTCCAGATAATGGAGCAGTGGCTGTATTAAAATACAACGGCATAATAACTGAAACCATAAAAAGTTGGA
GGAAGAAAATATTGAGGACACAAGAGTCTGAATGTGCCTGTGTAAATGGTTCATGTTTTACTATAATGACT
GATGGCCCGAGTGATGGGCTGGCCTCGTACAAAATTTTCAAGATCGAAAAGGGGAAGGTTACTAAATCA
ATAGAGTTGAATGCACCTAATTCTCACTATGAGGAATGTTCCTGTTACCCTGATACCGGCAAAGTGATGT
GTGTGTGCAGAGACAATTGGCATGGTTCGAACCGGCCATGGGTGTCTTTCGATCAAAACCTGGATTATC
AAATAGGATACATCTGCAGTGGGGTTTTCGGTGACAACCCGCGTCCCGAAGATGGAACAGGCAGCTGTG
GTCCAGTGTATGTTGATGGAGCAAACGGAGTAAAGGGATTTTCATATAGGTATGGTAATGGTGTTTGGAT
AGGAAGGACCAAAAGTCACAGTTCCAGACATGGGTTTGAGATGATTTGGGATCCTAATGGATGGACAGA
GACTGATAGTAAGTTCTCTGTGAGGCAAGATGTTGTGGCAATGACTGATTGGTCAGGGTATAGCGGAAG
TTTCGTTCAACATCCTGAGCTGACAGGGCTAGACTGTATGAGGCCGTGCTTCTGGGTTGAATTAATCAGG
GGACGACCTAAAGAAAAAACAATCTGGACTAGTGCGAGCAGCATTTCTTTTTGTGGCGTGAATAGTGATA
CTGTAGATTGGTCTTGGCCAGACGGTGCTGAGTTGCCATTCAGCATTGACAAGTAGTCTGTTCAAAAAAC
TCCTTGTTTCTACT

High-titer A/PR/8/34 (H1N1, PR8 (UW) virus grows 10 times better than other A/PR/8/34 PR8 strains in eggs (10¹⁰ EID₅₀/mL; HA titer: 1:8,000). Thus, replacement of the HA and NA genes of PR8(UW) with those of a currently circulating strain of influenza virus results in a vaccine strain that can be safely produced, and validates the use of PR8(UW) as a master vaccine strain.

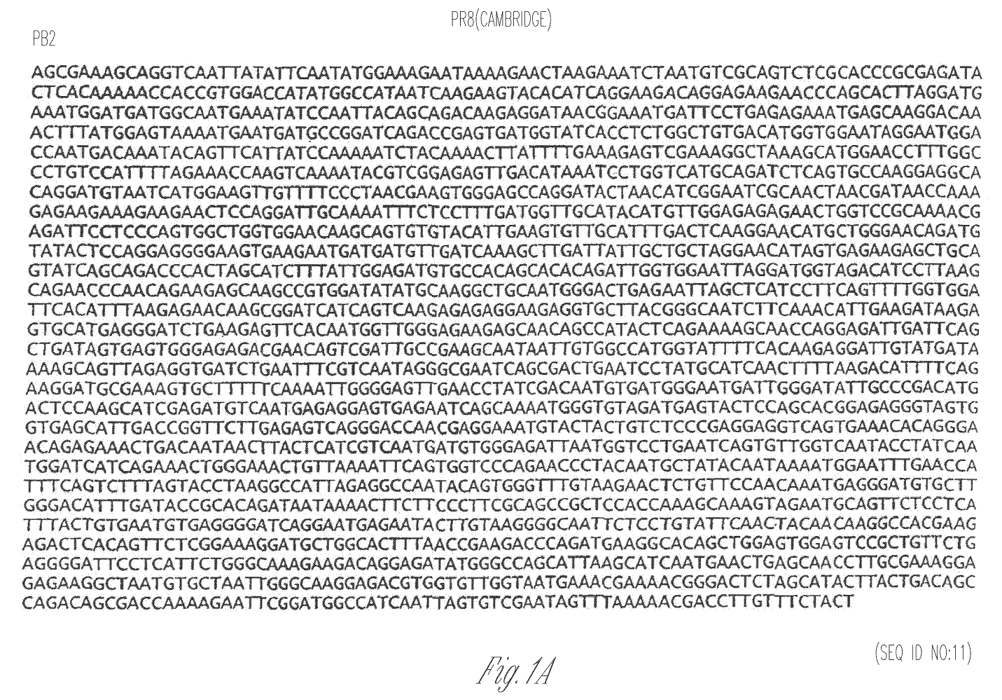

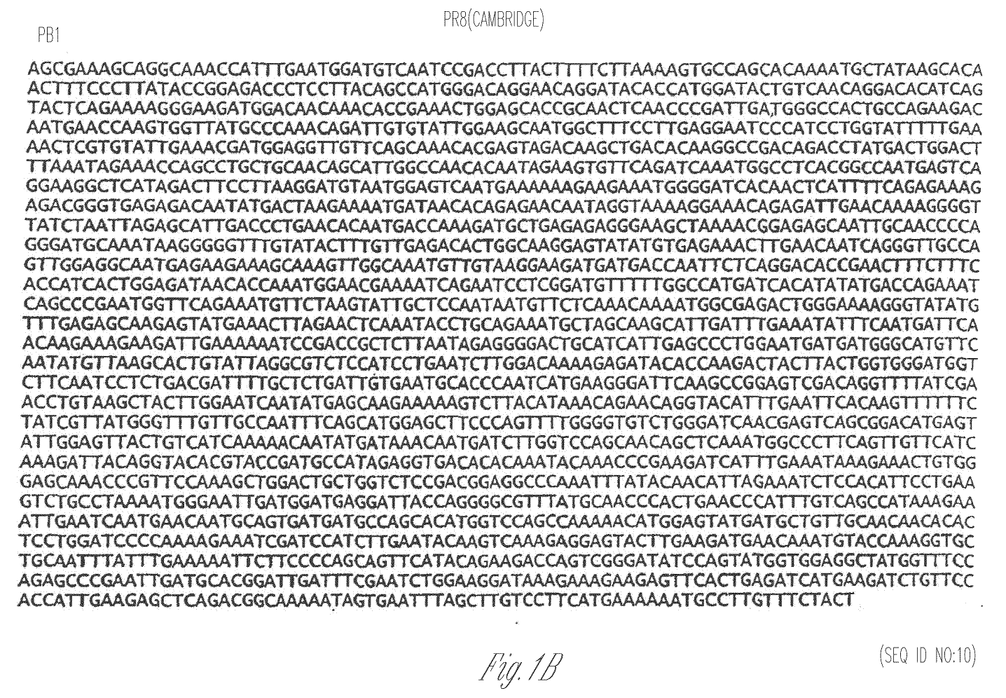

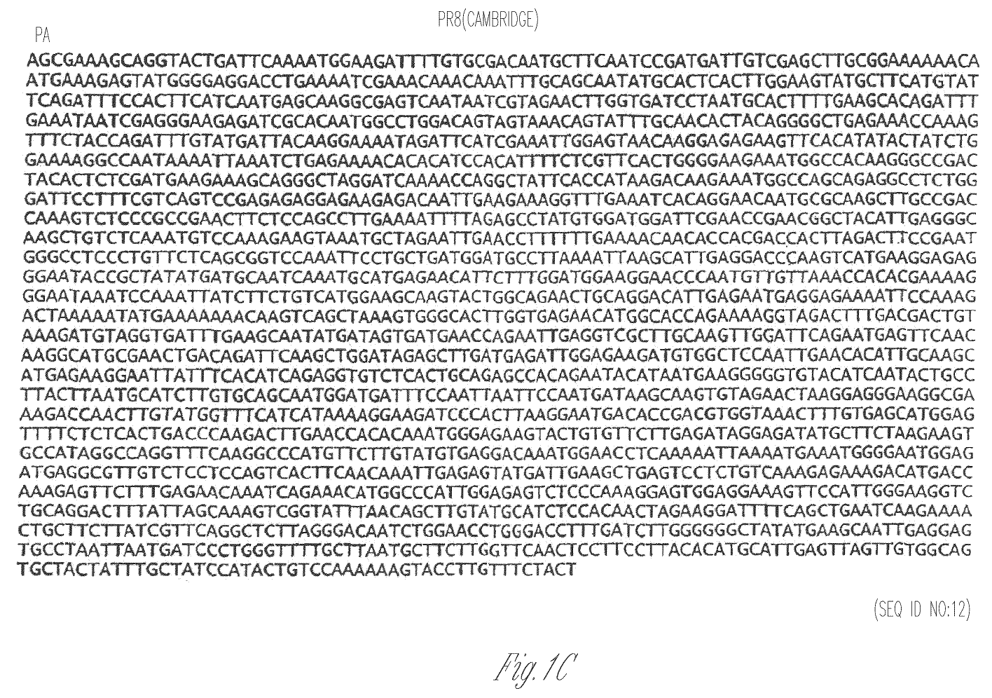

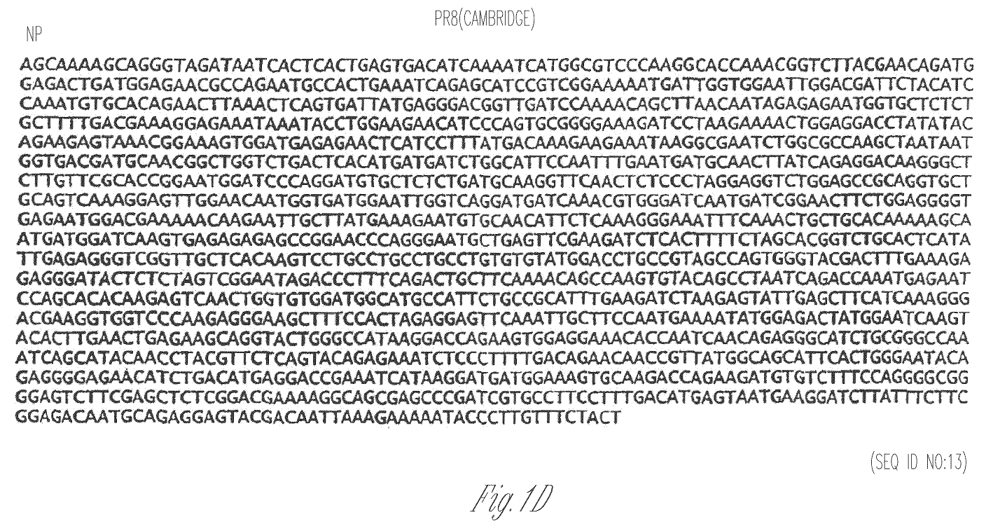

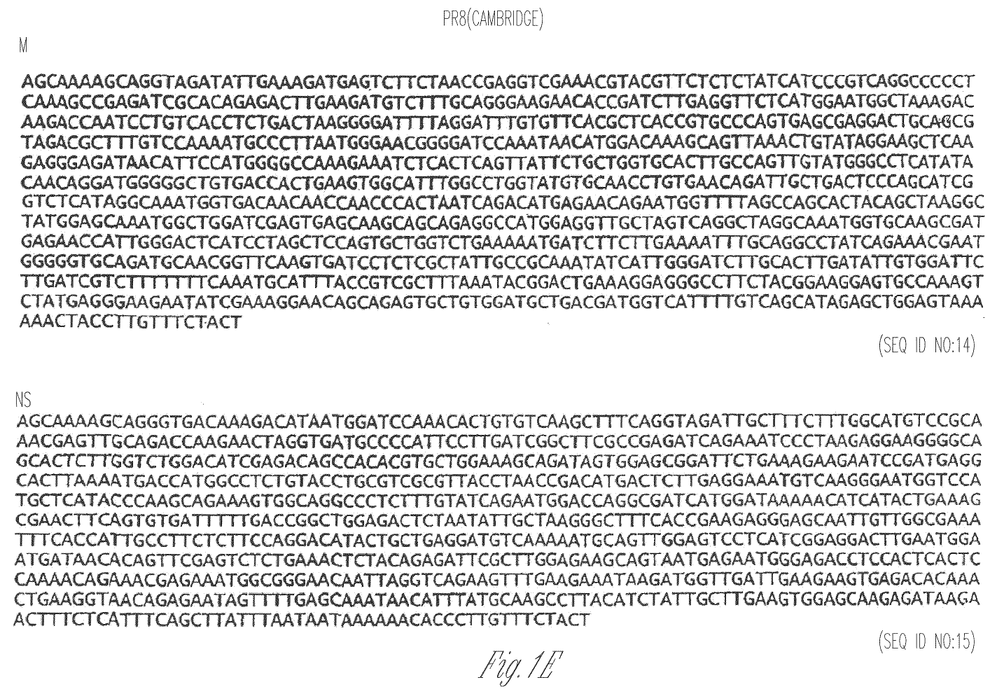

Genes that contribute to different growth properties between PR8(UW) and PR8 (Cambridge), which provides the non-HA and-NA genes of the NIBRG-14 vaccine strain (FIG. 1), were determined. Higher titers in eggs were obtained when the majority of internal genes were from PR8(UW). Highest titers were with the M viral segment of PR8(UW) and the NS gene of PR8 (Cambridge). The NS gene in PR8(UW) has a K (lysine) at residue 55 while the NS gene in PR8(Cam) has a E (glutamic acid). The polymerase subunit (PA, PB1, and PB2) and NP genes of PR8(UW) enhanced the growth of an H5N1 vaccine seed virus in chicken embryonated eggs, and the NS gene of PR8(Cambridge) enhanced the growth of an H5N1 vaccine seed virus in chicken embryonated eggs. A tyrosine (Y) at position 360 in PB2 of PR8(UW) likely contributes to the high growth rate of that virus in MDCK cells.

### EXAMPLE 2

To develop an high-yield A/PR/8/34 (H1N1; PR8) virus backbone for growth of vaccine virus in specific host cells, random mutagenesis of the internal genes of PR8(HG) (PR8UW) was conducted. Random mutations were introduced into the UW-PR8 (Example 1) internal genes by error-prone PCR, after which plasmid libraries were prepared that possessed the random mutations in an individual UW-PR8 internal gene. Then virus libraries (PR8/H5N1) were generated that possessed random mutations in an individual UW-PR8 internal gene, along with the other wild type internal genes and the NA and ‘detoxified’ HA genes of A/chicken/Indonesia/NC/09 (H5N1) virus (Table 1), to generate “6+2” recombinant viruses. Consecutive passages of the virus in MDCK cells were employed to select for variants with high-growth properties.

TABLE 1
Virus libraries generated

| | Internal genes | | | Titer of virus |
|---|---|---|---|---|
| | | Other internal | | library |
| Number | Gene library | genes | HA + NA | (pfu/ml) |
| Control | PR8 wild type | | NC/09/H5N1 | 3 × 10⁶ |
| 1 | PB2 | 5 UW-PR8 genes | NC/09/H5N1 | 2.1 × 10² |
| 2 | PB1 | 5 UW-PR8 genes | NC/09/H5N1 | 1.6 × 10⁵ |
| 3 | PA | 5 UW-PR8 genes | NC/09/H5N1 | 7 × 10³ |
| 4 | NP | 5 UW-PR8 genes | NC/09/H5N1 | 1.5 × 10³ |
| S | M | 5 UW-PR8 genes | NC/09/H5N1 | 1 × 10⁶ |
| 6 | NS | 5 UW-PR8 genes | NC/09/H5N1 | 1.8 × 10⁶ |
| 7 | PB2 + PB1 + PA | 3 UW-PR8 genes | NC/09/H5N1 | 75 |
| 8 | PB2 + PB1 + PA + NP | 2 UW-PR8 genes | NC/09/H5N1 | 33 |
| 9 | PB2 + NS | 4 UW-PR8 genes | NC/09/H5N1 | 2 × 10² |
| 10 | M + NS | 4 UW-PR8 genes | NC/09/H5N1 | 5.7 × 10⁵ |

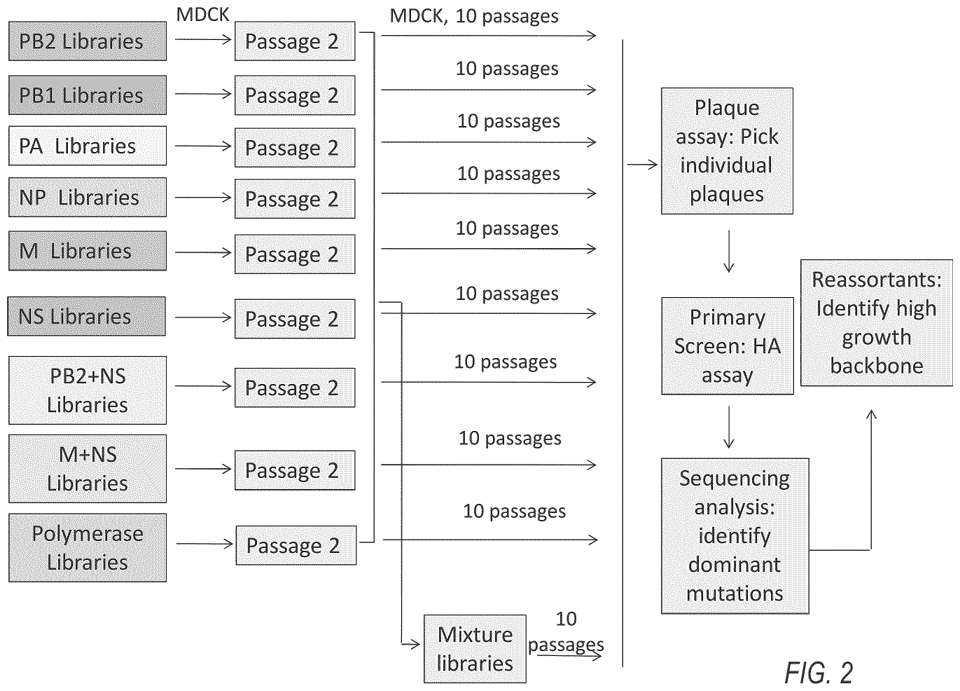

Virus libraries were passaged 12 times in MDCK cells or, after 2 passages, the libraries were mixed and 10 more passages were carried out (FIG. 2).

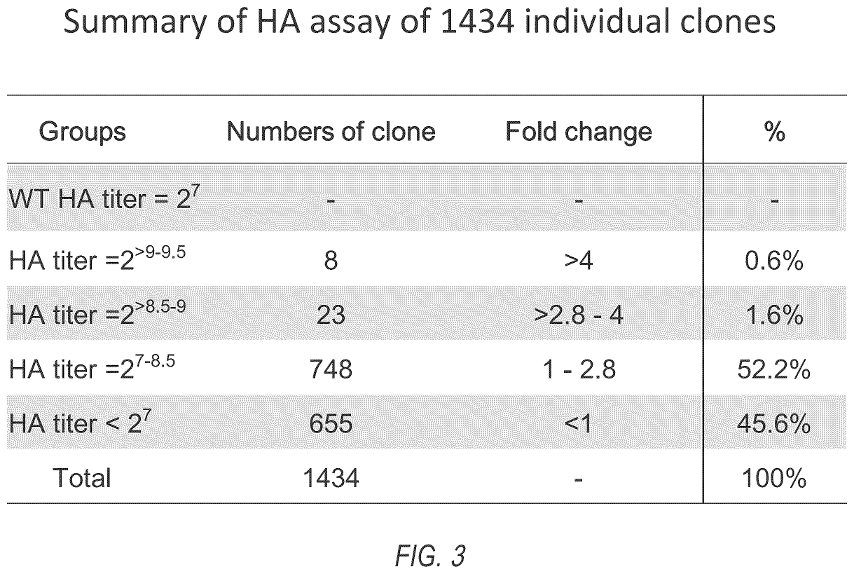

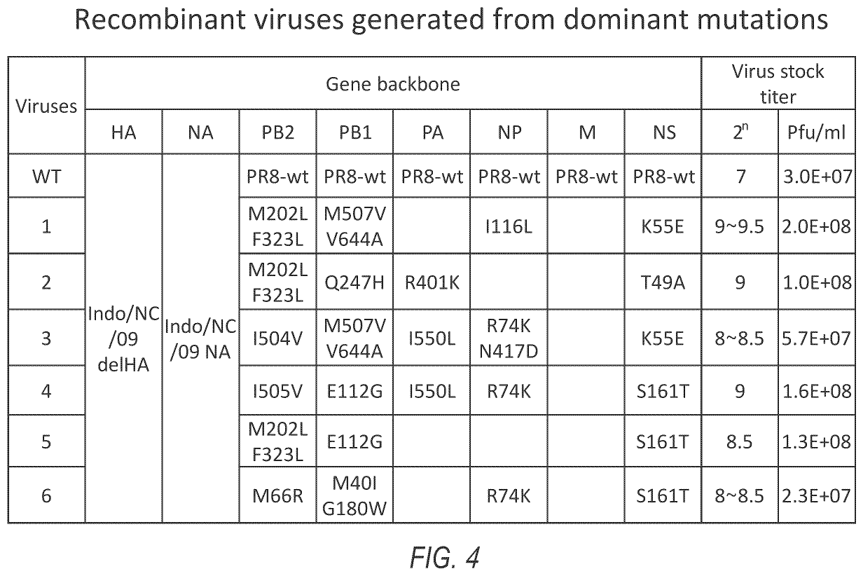

After 10 to about 12 consecutive passages in MDCK cells, plaque assays were performed and over 1,400 individual plaques were picked. FIG. 3 shows the numbers of clones with various HA titers. Growth-enhancing mutations included: PB2: M202L, F323L, I504V, PB1: E112G, V644A, NP: R74K, N417D, I116L, and NS1: S161T. FIG. 4 provides the titers of recombinant viruses generated from selected mutations.

36 viruses with the highest HA titers from the random mutagenesis libraries were sequenced (Table 2)

TABLE 2
Sequences of viruses with the highest HA titers

| | | HA | | | | | | | | |
|---|---|---|---|---|---|---|---|---|---|---|
| Clone | | titer | | | | HA (H3 | | | | |
| # | Library | (2ⁿ) | PB2 | PB1 | PA | numbering) | NP | NA | M | NS |
| WT | | 7 | | | | | | | | |
| 329 | Mix | 9 | M202L | | | L182V | | | | |
| | | | F323L | | | | | | | |
| 154 | Mix | 8.5~9 | M202L | | | L182V | | | | |
| | | | F323L | | | | | | | |
| 347 | Mix | 9 | M202L | | | L182V | | | | |
| | | | F323L | | | | | | | |
| 94 | Mix | 8.5 | M202L | | | F252I | I116L | L55S | | |
| | | | F323L | | | | | | | |
| 1045 | Mix | 9 | M202L | V644A | | F252I | | | | |
| | | | F323L | | | | | | | |
| 965 | Mix | 8.5~9 | M202L | | F105C | V184I | | | P90S | |
| | | | F323L | | | | | | | |
| 50 | Mix | 8.5 | M202L | | | M148I | R293M | | | |
| | | | F323L | | | (HA2) | | | | |
| 1005 | Mix | 9~9.5 | M202L | V644A | R401K | M148I | | | | T49A |
| | | | F323L | | | (HA2) | | | | |
| 134 | Mix | 8.5 | M202L | | | | | | | A223E |
| | | | F323L | | | | | | | |
| 387 | Mix | 9 | M202L | M507V | | | | | | |
| | | | F323L | V644A | | | | | | |
| 852 | Mix | 9~9.5 | M202L | R54I | | | | | | |
| | | | F323L | | | | | | | |
| | | | M243I | | | | | | | |
| 981 | Mix | 8.5~9 | M202L | Q247H | | | | | | |
| | | | F323L | | | | | | | |
| 993 | Mix | 8.5~9 | M202L | | | | N224I | | | |
| | | | F323L | | | | | | | |
| 1043 | Mix | 8.5~9 | I504V | | | L182V | R74K | | | |
| 398 | Mix | 8.5 | I504V | | | L182V | R74K, | | | A30P |
| | | | | | | | N417D | | | |
| 1007 | Mix | 8.5 | I504V | V644A | | F252I | M371V | | | |
| 1042 | Mix | 8.5~9 | I504V | E75V | | F252I | R74K | | | |
| | | | | D76G | | | | | | |
| | | | | E78P | | | | | | |
| | | | | P79V | | | | | | |
| | | | | S80G | | | | | | |
| | | | | V644A | | | | | | |
| | | | | E697P | | | | | | |
| | | | | F699L | | | | | | |
| | | | | F700L | | | | | | |
| | | | | P701H | | | | | | |
| | | | | S702R | | | | | | |
| | | | | Y705T | | | | | | |
| 999 | Mix | 8.5~9 | I504V | | | M148I | R74K, | | | |
| | | | | | | (HA2) | N417D | | | |
| 1014 | Mix | 8.5 | I504V | T59I | | M148I | R74K, | A265V | | |
| | | | | G62X | | (HA2) | N417D | | | |
| | | | | A63P | | | | | | |
| | | | | V644A | | | | | | |
| | | | | N694K | | | | | | |
| | | | | L695T | | | | | | |
| 1016 | Mix | 8.5~9 | I504V | | | M148I | | | | |
| | | | | | | (HA2) | | | | |
| 540 | PB1 | 8.5 | | E112G | | K162E | | | | S161T |
| | | | | (PB1-F2- | | | | | | |
| | | | | R81G) | | | | | | |
| 548 | PB1 | 8.5~9 | | E112G | | K162E | | | | S161T |
| | | | | (PB1-F2- | | | | | | |
| | | | | R81G) | | | | | | |
| | | | | L624V | | | | | | |
| 191 | PB1 | 8~8.5 | | E112G | | | | | | |
| | | | | (PB1-F2- | | | | | | |
| | | | | R81G) | | | | | | |
| 571 | PB1 | 9~9.5 | | E112G | | | | | | |
| | | | | (PB1-F2- | | | | | | |
| | | | | R81G) | | | | | | |
| 572 | PB1 | 8.5 | | E112G | | | | | | |
| | | | | (PB1-F2- | | | | | | |
| | | | | R81G) | | | | | | |
| 573 | PB1 | 8.5 | | E112G | | | | | | |
| | | | | (PB1-F2- | | | | | | |
| | | | | R81G) | | | | | | |
| 1404 | PB1 | 8.5 | I57V | E112G | | | | | | |
| | | | T58G | (PB1-F2- | | | | | | |
| | | | A59V | R81G) | | | | | | |
| | | | K61Q | S713C | | | | | | |
| | | | E677D | | | | | | | |
| | | | D678E | | | | | | | |
| | | | P679M | | | | | | | |
| 1408 | PB1 | 8.5 | | M40I | | | | | | S161T |
| | | | | G180W | | | | | | |
| 582 | PB1 | 8.5~9 | | M40L, | | | | | | S161T |
| | | | | G180W | | | | | | |
| 545 | PB1 | 8.5 | | M40L, | | K121E | | | | |
| | | | | G180W | | (HA2) | | | | |
| 543 | PB1 | 8.5 | | I667T | | | | | | |
| 219 | PB1 | 9 | | I667T, | | K162E | | | | |
| | | | | M714T | | | | | | |
| 344 | Mix | 8.5~9 | M66R | | | L182V | | | | |
| 312 | Mix | 8.5~9 | | | | L182V | I116L | | | R140Q |
| 320 | Mix | 8.5 | | | | L182V | | | | |
| 209 | PB1 | 8.5~9 | | R54I | | E136D, | | | | |
| | | | | | | Q179L, | | | | |
| | | | | | | A194V | | | | |

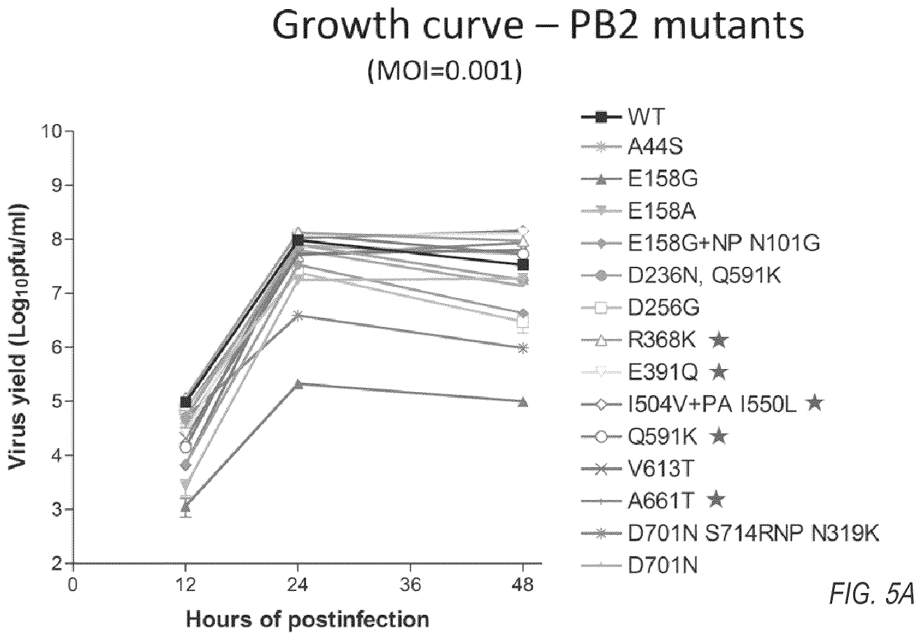

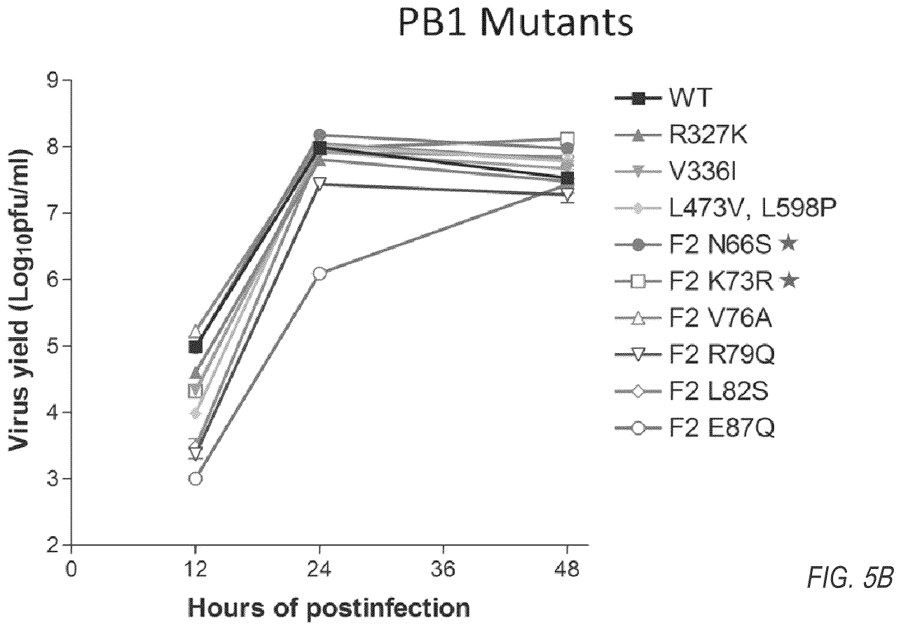

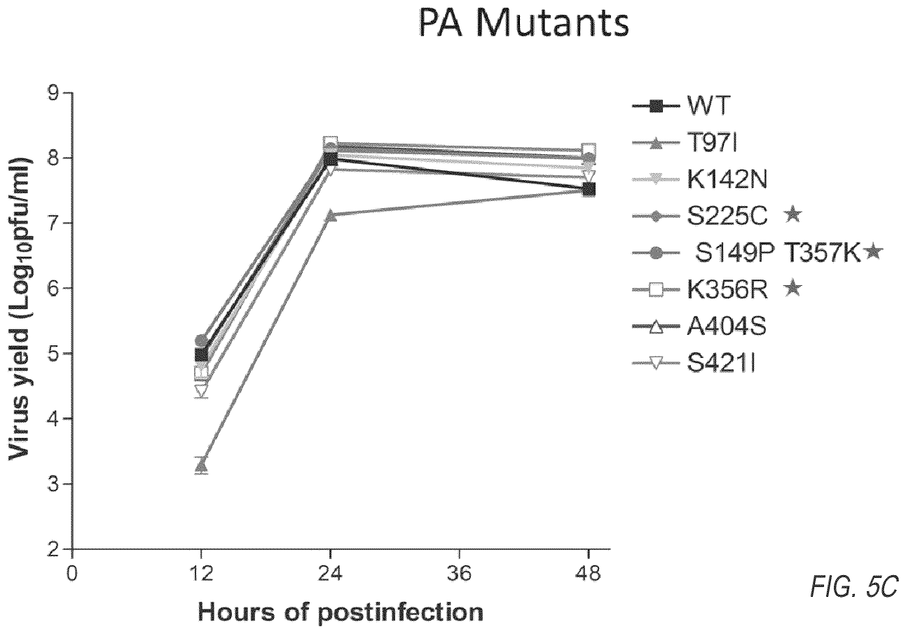

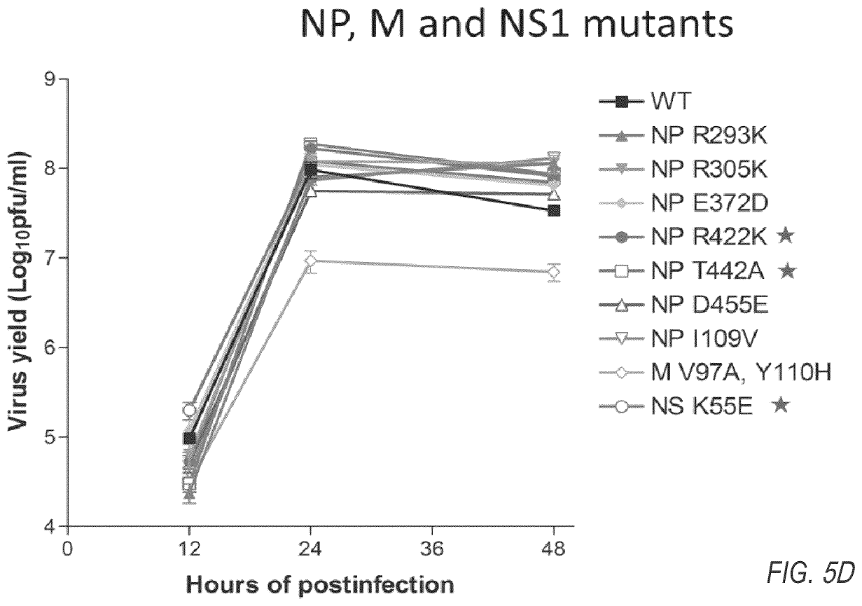

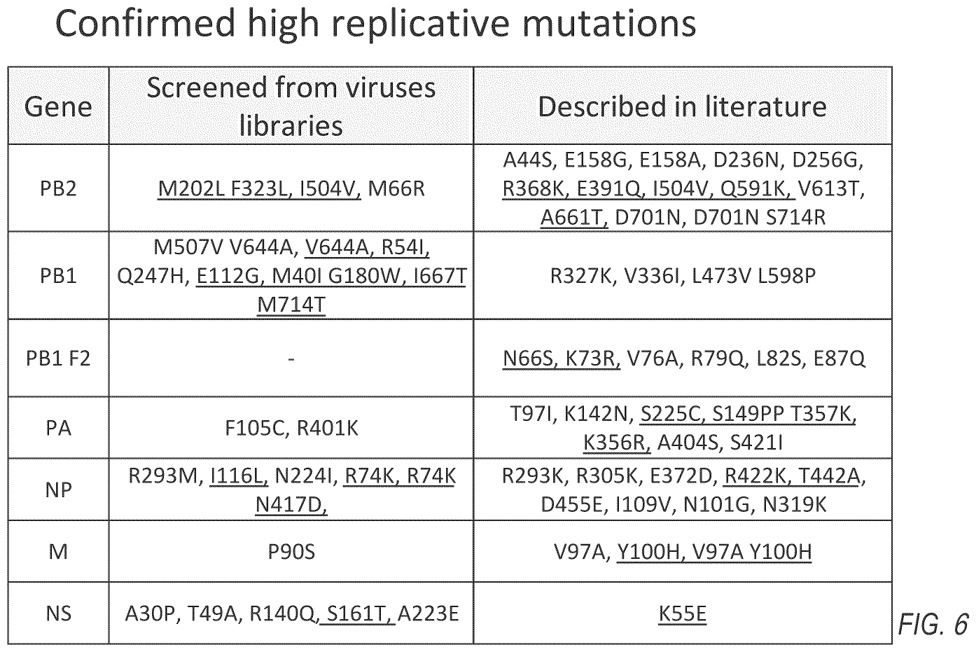

In a second approach, potentially growth-enhancing mutations described in the literature were introduced into the background of UW-PR8 virus (see Table 3 for virus stock titers) and tested for replicative ability. FIGS. 5A-D show growth curves for various viruses.

TABLE 3
UW-PR8 viruses possessing mutation(s)
identified in the literature

| | | Virus stock |
|---|---|---|
| Gene | Mutation(s) | titer (Pfu/ml) |
| WT | — | 2 × 10⁷ |
| PB2 | A44S | 4.5 × 10⁷ |
| | E158G | 3.2 × 10⁴ |
| | E158G + NP N101G | 7.5 × 10⁴ |
| | E158A | 8.3 × 10⁶ |
| | D253N + Q591K | 8.3 × 10⁶ |
| | D256G | 2.8 × 10⁷ |
| | R368K | 3.1 × 10⁷ |
| | E391Q | 1.4 × 10^{s} |
| | 1504V + PA I550L | 1.1 × 10^{s} |
| | Q591K | 4.4 × 10⁷ |
| | V613T | 1.8 × 10⁷ |
| | A661T | 2.2 × 10⁷ |
| | D701N + S714R + NP N319K | 1 × 10⁶ |
| | D701N | 2.1 × 10⁷ |
| PB1 | R327K | 1.3 × 10⁷ |
| | V336I | 2.3 × 10⁷ |
| | L473V+ L598P | 3.9 × 10^{s} |
| WT | — | 2 × 10⁷ |
| PB1F2 | F2 N66S | 1.6 × 10^{s} |
| | F2 K73R | 1.1 × 10^{s} |
| | F2 V76A | 4.4 × 10⁷ |
| | F2 R79Q | 6.2 × 10^{s} |
| | F2 L82S | 2.7 × 10⁷ |
| | F2 E87Q | 1.5 × l0⁶ |
| PA | T97I | 1.6 × 10⁷ |
| | K142N | 3.3 × 10⁷ |
| | S225C | 6.7 × 10⁷ |
| | S149P + T357K | 3.4 × 10⁸ |
| | K356R | 8.5 × 10⁷ |
| | A404S | 5.2 × 10⁷ |
| | S421I | 2.7 × 10⁷ |
| NP | R293K | 4.7 × 10⁷ |
| | R305K | 7.2 × 10⁷ |
| | E372D | 2.2 × 10⁷ |
| | R422K | 1.3 × 10^{s} |
| | T442A | 5 × 10⁷ |
| | D455E | 2.2 × 10⁷ |
| | I109V | 3.9 × 10⁷ |
| M | V97A + Y100H | 1.4 × 10⁷ |
| NS1 | K55E | 1.6 × 10⁷ |

In a third approach, candidates from approaches 1 and 2 were combined and HA titers and PFU/mL determined (Table 4).

TABLE 4
High-growth candidates identified in approaches 1 and 2 were tested in various combinations.

| | | | | | | | | | Virus stock titer | |
|---|---|---|---|---|---|---|---|---|---|---|
| | Gene origin | | | | | | | | HA | |
| # | HA | NA | PB2 | PB1 | PA | NP | M | NS | (2ⁿ) | Pfu/ml |
| WT | Indo/NC/09 | Indo/NC/09 | UW-PR8 | UW- | UW- | UW- | UW- | UW- | 7 | 3.00E+07 |
| | (detoxified) | | | PR8 | PR8 | PR8 | PR8 | PR8 | | |
| 1 | | | M202L | M507V | | I116L | | K55E | 9~9.5 | 2.00E+08 |
| | | | F323L | V644A | | | | | | |
| 2 | | | M202L | R54I | | N224I | | K55E | 5 | 1.00E+05 |
| | | | F323L | | | | | | | |
| 3 | | | M202L | Q247H | R401K | | | T49A | 9 | 1.00E+08 |
| | | | F323L | | | | | | | |
| 4 | | | M202L | M507V | K356R | T442A | V97A | K55E | 10~10.5 | 1.60E+08 |
| | | | F323L | V644A | | | Y100H | | | |
| 5 | | | I504V | M507V | I550L | R74K | | K55E | 8~8.5 | 5.70E+07 |
| | | | | V644A | | N417D | | | | |
| 6 | | | I504V | M507V | I550L | R74K | V97A | K55E | 9~9.5 | 4.40E+07 |
| | | | | V644A | | N417D | Y100H | | | |
| 7 | | | I505V | E112G | I550L | R74K | | S161T | 9 | 1.60E+08 |
| 8 | | | M202L | I667T | | I116L | | R140Q | <1 | <1E3 |
| | | | F323L | M714T | | | | | | |
| 9 | | | M202L | E112G | | | | S161T | 8.5 | 1.30E+08 |
| | | | F323L | (PB1-F2- | | | | | | |
| | | | | R81G) | | | | | | |
| 10 | | | M66R | M40I | | R74K | | S161T | 8~8.5 | 2.30E+07 |
| | | | | G180W | | | | | | |
| 12 | | | R368K | PB1 F2 | K356R | R422K | | K55E | 5.5 | 9.00E+02 |
| | | | | N66S | | | | | | |
| 13 | | | E391Q | R327K | S149P | R293K | | | 3 | 1.60E+06 |
| | | | | | T357K | | | | | |
| 14 | | | Q591K | PB1 F2 | S225C | R422K | | K55E | 7.5 | 2.00E+07 |
| | | | | K73R | | | | | | |
| 23 | | | | | | | V97A | | 8.5~9 | 1.50E+07 |
| 24 | | | | | | | Y100H | | 9~9.5 | 2.90E+07 |
| 25 | NCR 15- | Indo/NC/09 | M202L | M507V | K356R | R422K | V97A | K55E | 9.5~10 | 7.50E+07 |
| | 19 nt mut¹ | | F323L | V644A | | | Y100H | | | |
| 26 | Indo/NC/09 | Indo/NC/09 | | | | | | A30P | 6.5~7 | 1.00E+07 |
| 27 | (detoxified) | | | | | | | T49A | 6.5~7 | 2.00E+07 |
| 28 | | | | | | | | R140Q | 8 | 4.00E+07 |
| 29 | | | | | | | | S161T | 7~7.5 | 1.40E+07 |
| 30 | | | | | | | | A223E | 7.5 | 1.00E+07 |
| 31 | | | | I667T | | | | | 3.5 | 4.00E+05 |
| | | | | M714T | | | | | | |
| 32 | NCR 15- | UW-PR8 | M202L | V644A | K356R | T442A | Y100H | K55E | 7~7.5 | 4.30E+06 |
| | 19 nt mut | | F323L | | | | | | | |
| 33 | Indo/NC/09 | Indo/NC/09 | M202L | E112G | K356R | R74K | Y100H | K55E | 9~9.5 | 7.00E+07 |
| | (detoxified) | | F323L | (PB1-F2- | | | | | | |
| | | | | R81G) | | | | | | |
| 34 | NCR 15- | UW-PR8 | I504V | M507V | | | V97A | K55E | 7 | 2.00E+05 |
| | 19 nt mut | | | V644A | | | Y100H | | | |
| 35 | Indo/NC/09 | Indo/NC/09 | M202L | M507V | R401K | T442A | Y100H | R140Q | 9 | 3.20E+07 |
| | (detoxified) | | F323L | V644A | | | | | | |
| 36 | | | I504V | E112G | I550L | I112L | Y100H | R140Q | 9.5 | 1.30E+08 |
| | | | | (PB1-F2- | | | | | | |
| | | | | R81G) | | | | | | |
| 37 | | | M202L | E112G | S149P | T442A | Y100H | K55E | 0 | 0.00E+00 |
| | | | F323L | (PB1-F2- | T357K | | | | | |
| | | | | R81G) | | | | | | |
| 38 | | | M202L | M507V | | I116L | Y100H | K55E | 10.1 | 2.30E+08 |
| | | | F323L | V644A | | | | | | |
| 39 | | | M202L | M507V | K356R | T442A | Y100H | K55E | 9.8 | 1.00E+08 |
| | | | F323L | V644A | | | | | | |
| 40 | | | I504V | M507V | I550L | T442A | Y100H | K55E | 9.2 | 6.00E+07 |
| | | | | V644A | | | | | | |
| 41 | | | I504V | I112G | I550L | R74K | Y100H | K55E | 9.2 | 7.50E+07 |
| P17 | | | I504V | E112G | S225C | R74K | V97A | K55E | 9.5~10 | 5.80E+08 |
| | | | | (PB1-F2- | | N417D | Y100H | | | |
| | | | | R81G) | | | | | | |
| P26 | | | M202L | M40L | S225C | R422K | V97A | K55E | 10 | 3.00E+08 |
| | | | F323L | G180W | | | Y100H | | | |
| P61 | | Indo/NC/09 | M202L | Q247H | K142N | R74K | V97A | K55E | 10~10.5 | 2.00E+08 |
| | | NA P263T² | F323L | | | | Y100H | | | |

¹Mutation in the HA gene noncoding region;
²A P263T mutation was detected in the NA protein of this virus clone

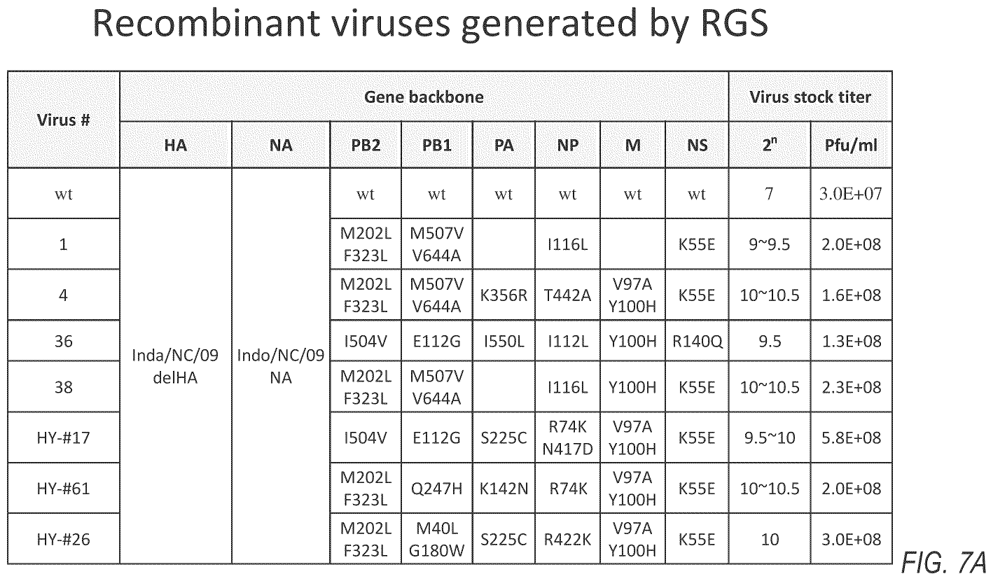

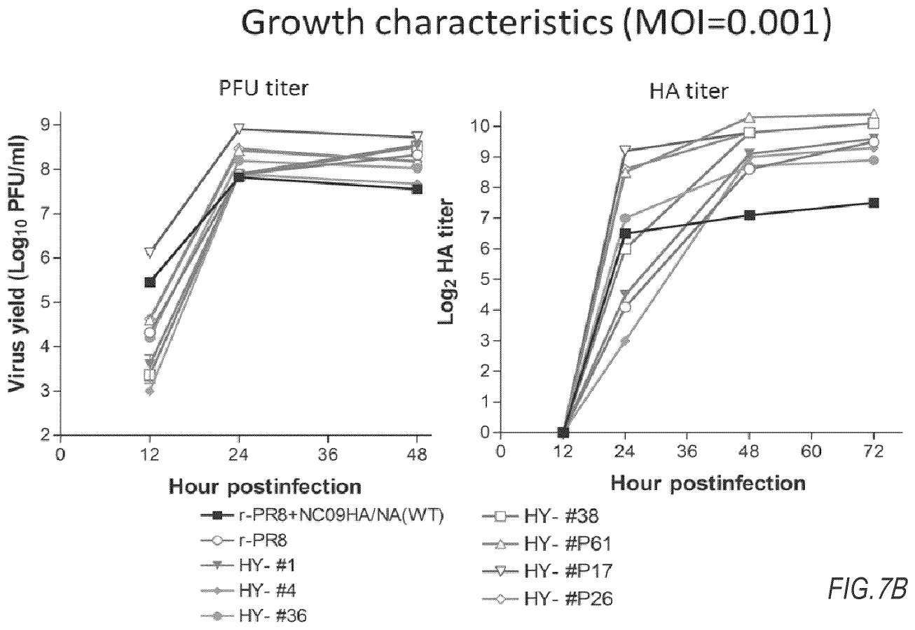

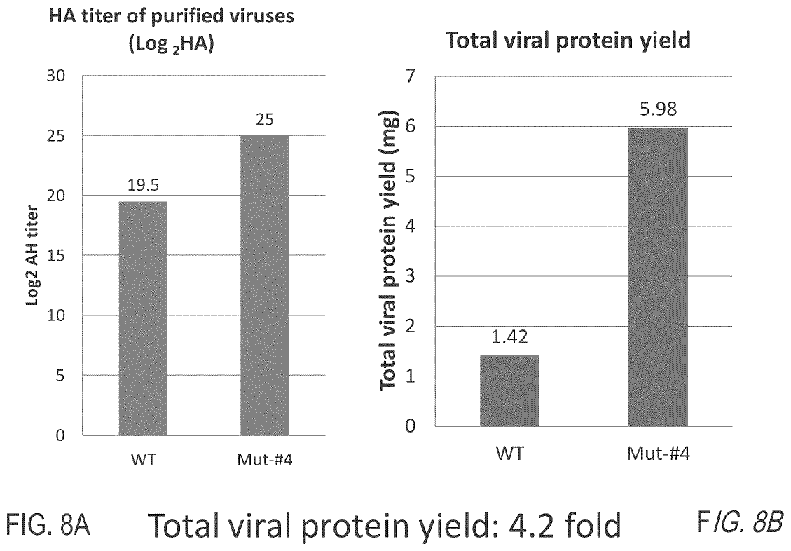

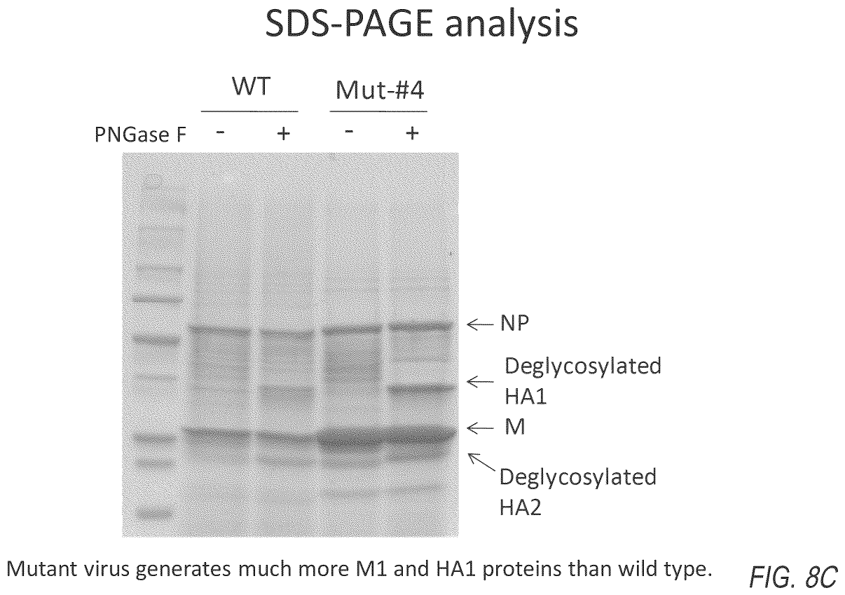

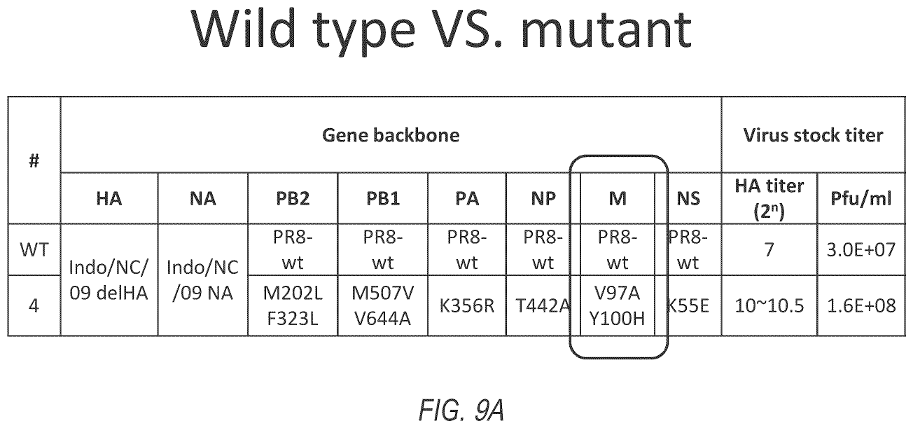

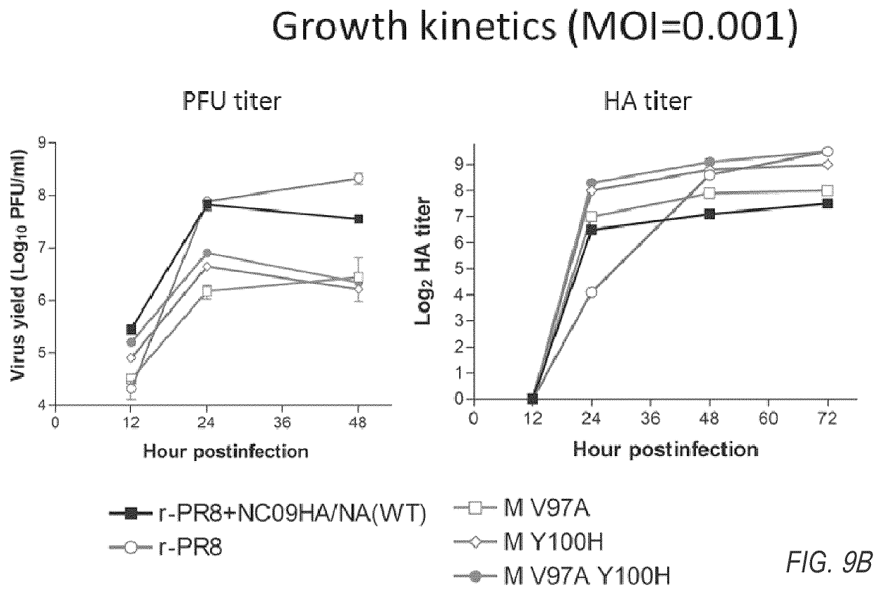

As shown in Table 4, several recombinant viruses were identified that replicated better than wild type, such as #1, #4, #36, #38, P17, P16, and P61. To identify the growth characteristics of these viruses, growth kinetics in MDCK cells were determined (FIG. 7). For one candidate, virus was purified on sucrose gradients and HA content and viral total protein evaluated. FIG. 8A shows HA titer of wild type (UW-PR8) and #4, FIG. 8B shows viral protein for wild type (UW-PR8) and #4, and FIG. 8BC is a SDS-PAGE analysis of viral proteins of wild type (UW-PR8) and #4. Further analysis demonstrated that viruses possessing the V97A/Y100H mutations in M1 yielded higher HA titers than the parental virus, although the virus titer was lower (see FIGS. 9A-B). The V97A/Y100H mutations in M1 may result in particles with a larger surface into which more HA protein can be incorporated. Since inactivated influenza viruses are dosed based on their HA content, variants with high HA content are attractive vaccine candidates.

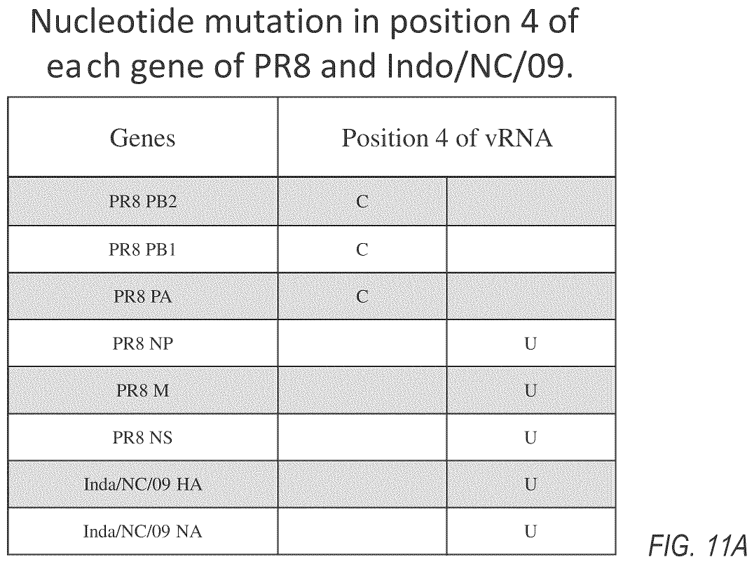

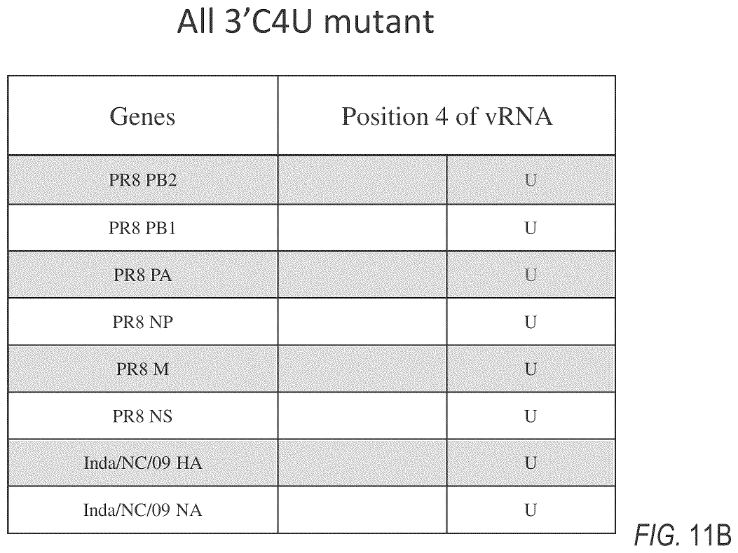

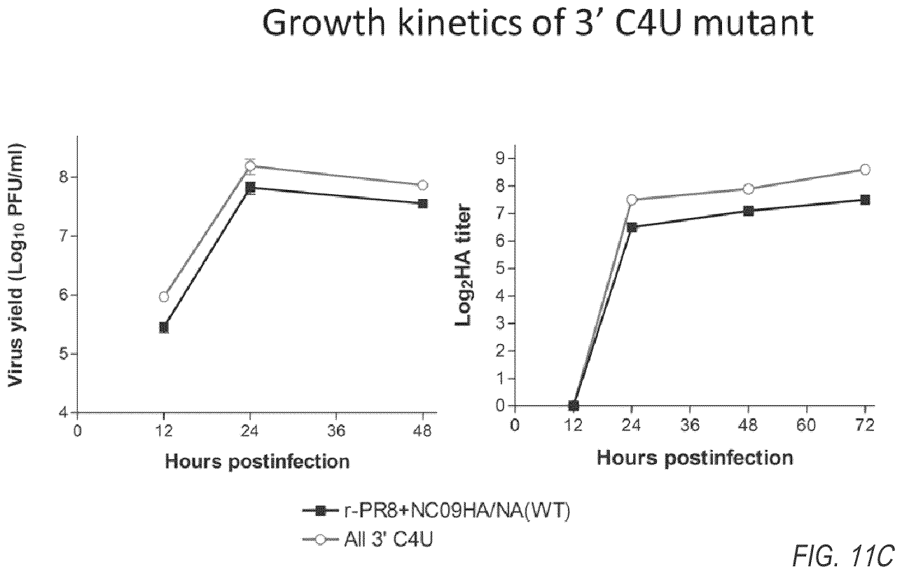

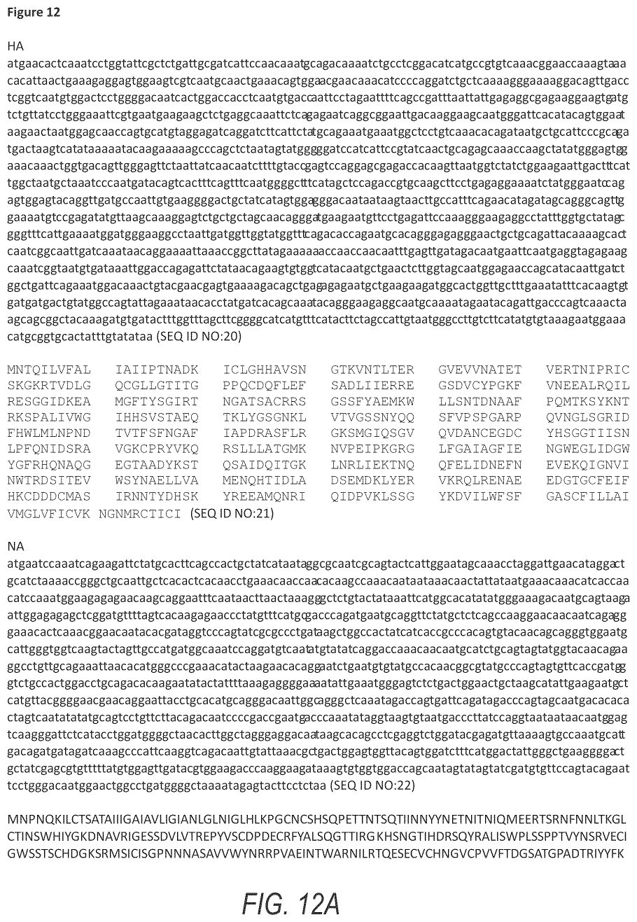

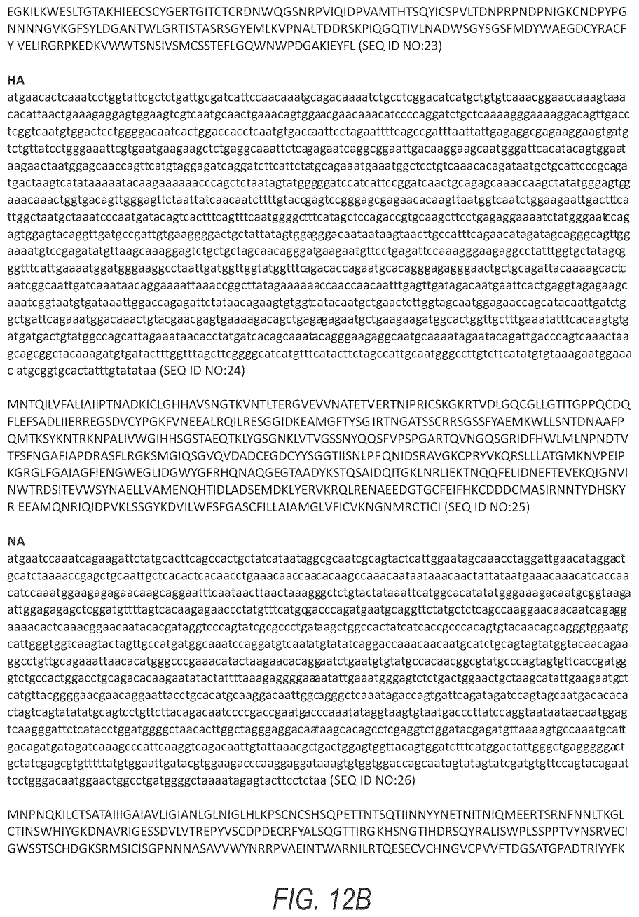

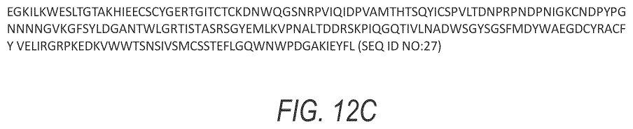

To identify mutations in the influenza promoter region that provide for enhanced replication, viruses possessing a ‘U’ at position 4 at the 3′ end of all eight vRNA segments were prepared in the UW-PR8 PA, PB1 and PB2 internal genes (the UW-PR8 PB2, PB1, and PA segments possess a ‘C’ at position 4). The growth curves of the resulting viruses are shown in FIG. 11C.

Viruses possessing combinations of promoter mutations and amino acid changes were prepared and titers determined (Table 5).

| | | | | | | | | | Virus stock titer | |
|---|---|---|---|---|---|---|---|---|---|---|
| | Gene backbone | | | | | | | | HA | |
| Virus | HA | NA | PB2 | PB1 | PA | NP | M | NS | (2ⁿ) | pfu/ml |
| Wild type | WT | WT | WT | WT | WT | WT | WT | WT | 7~7.5 | 3.5E+07 |
| PB2 codon | WT | WT | Rare | WT | WT | WT | WT | WT | 9 | 2.1E+08 |
| optimization- | | | codon | | | | | | | |
| 1 | | | optimized | | | | | | | |
| | | | PB2 | | | | | | | |
| PB2 codon | WT | WT | All | WT | WT | WT | WT | WT | 3 | 9.0E+05 |
| optimization- | | | Codon | | | | | | | |
| 2 | | | optimized | | | | | | | |
| | | | PB2 | | | | | | | |

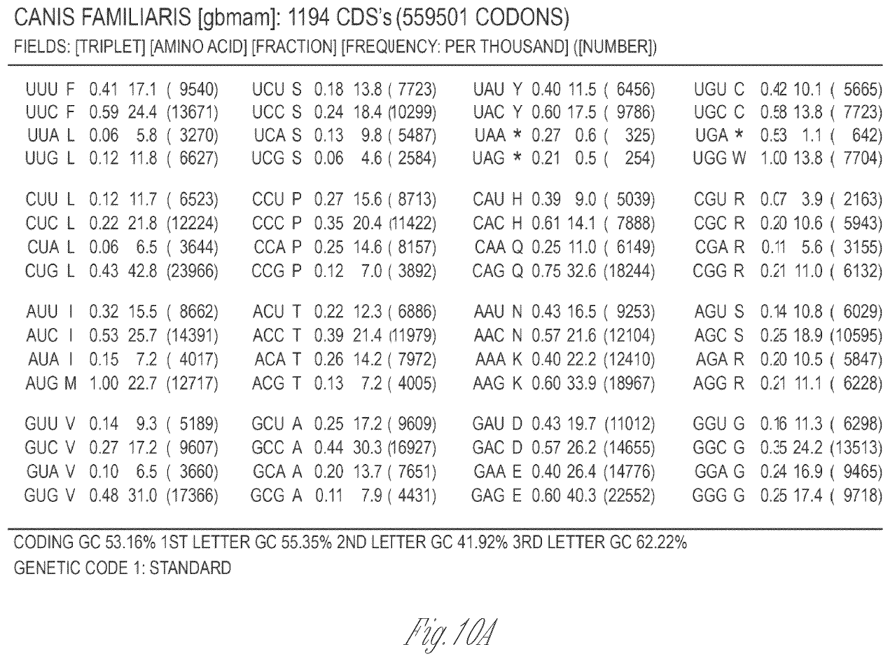

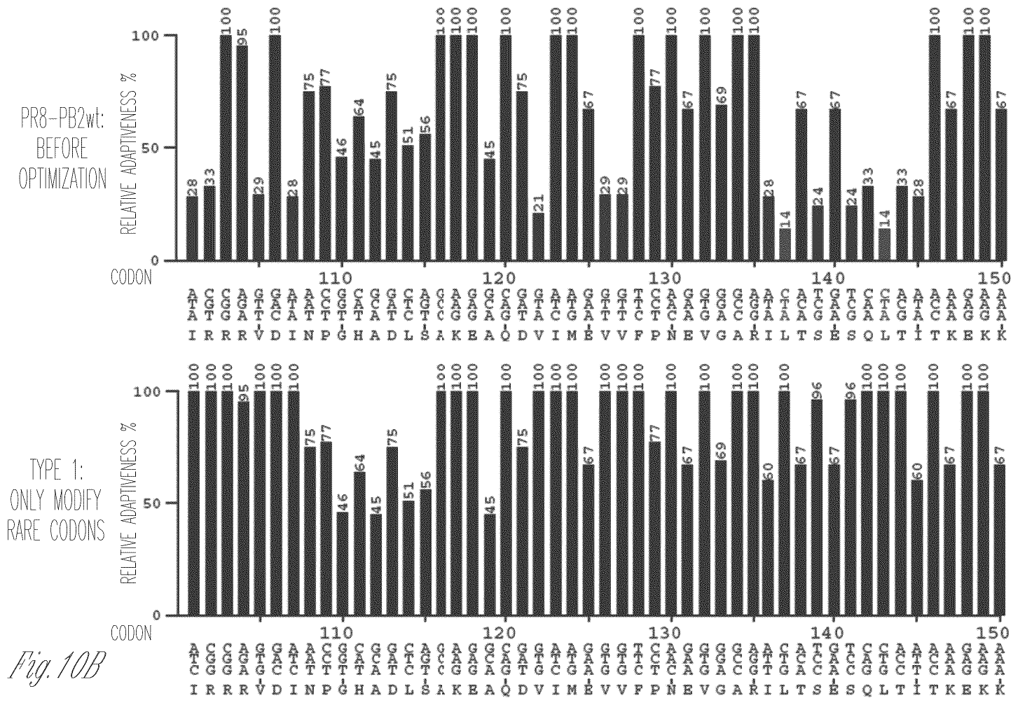

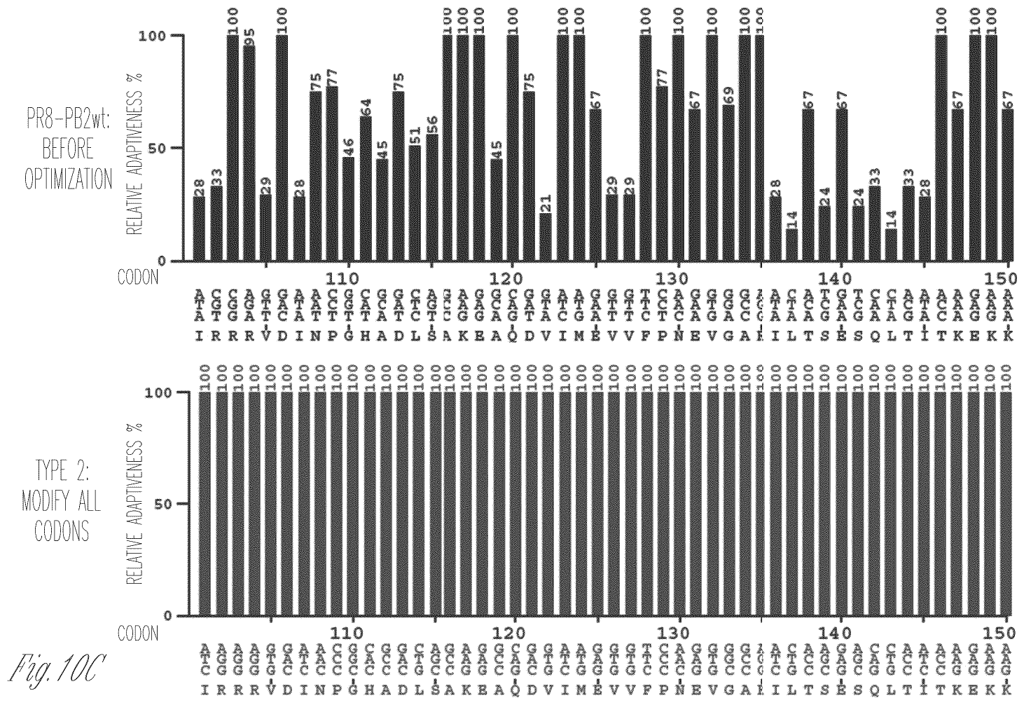

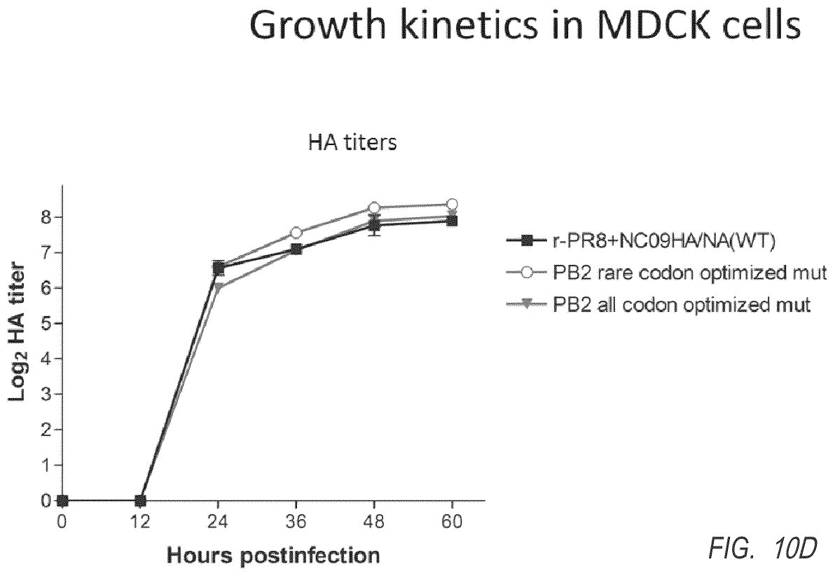

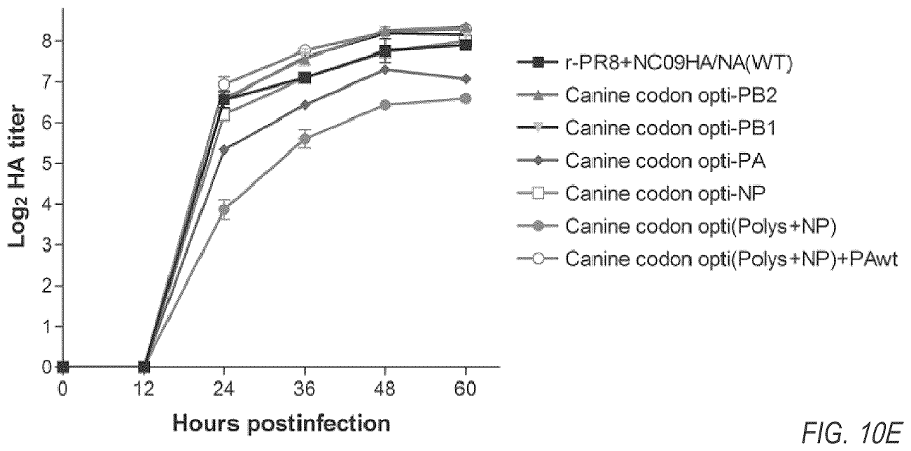

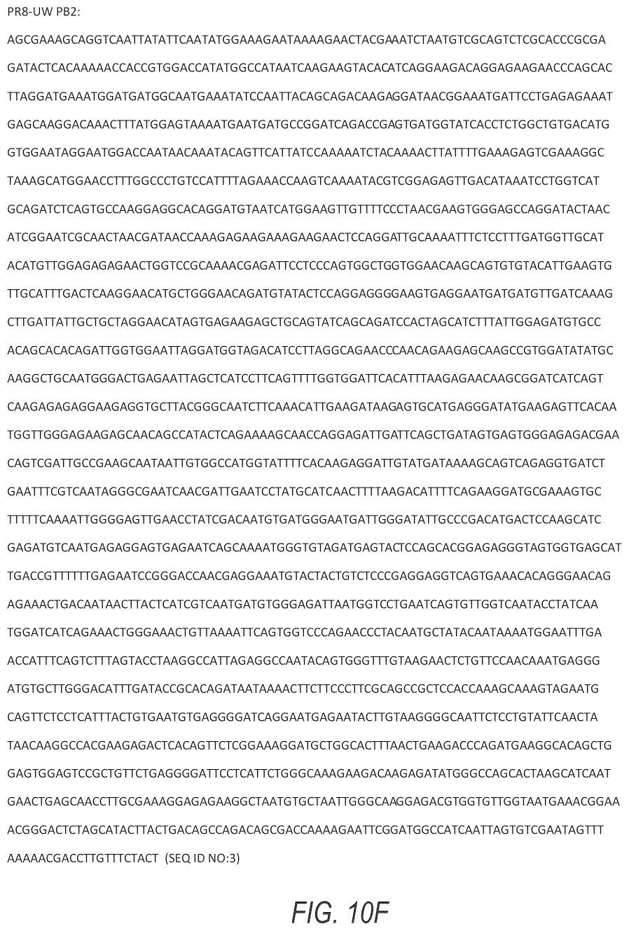

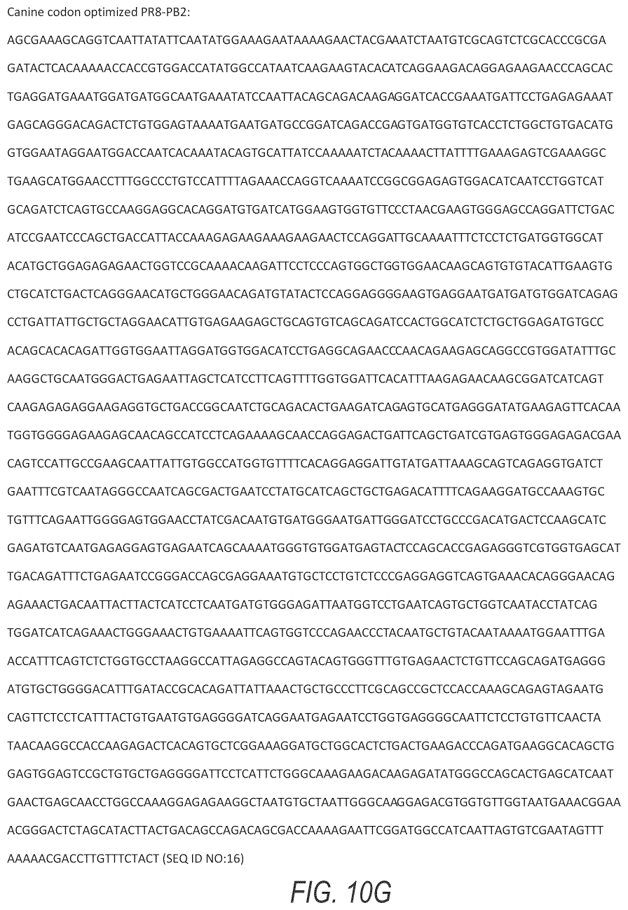

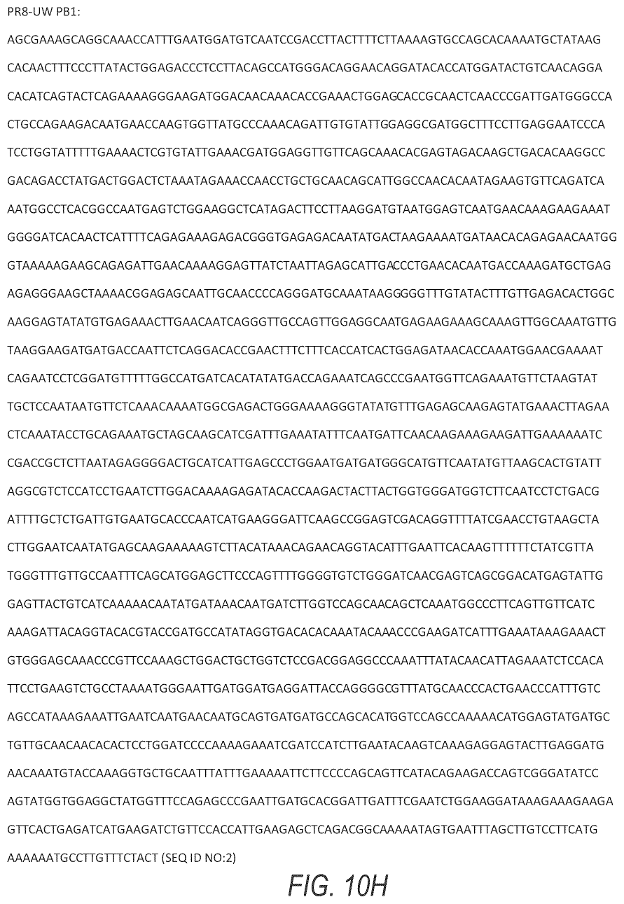

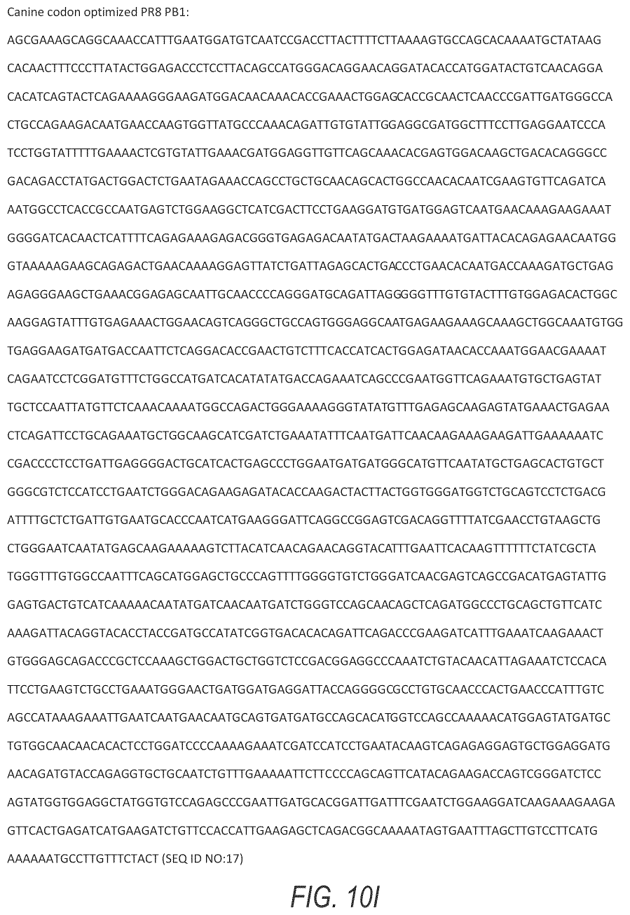

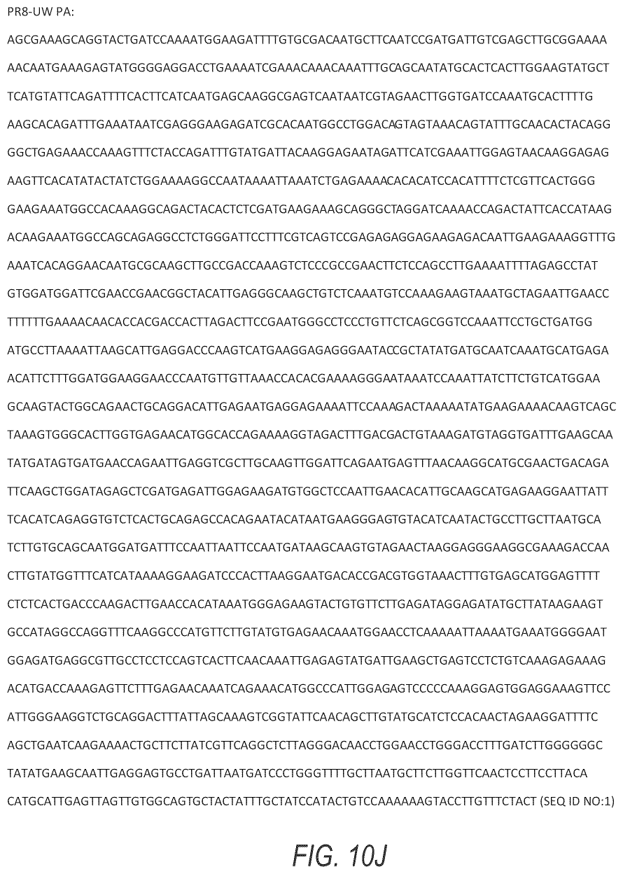

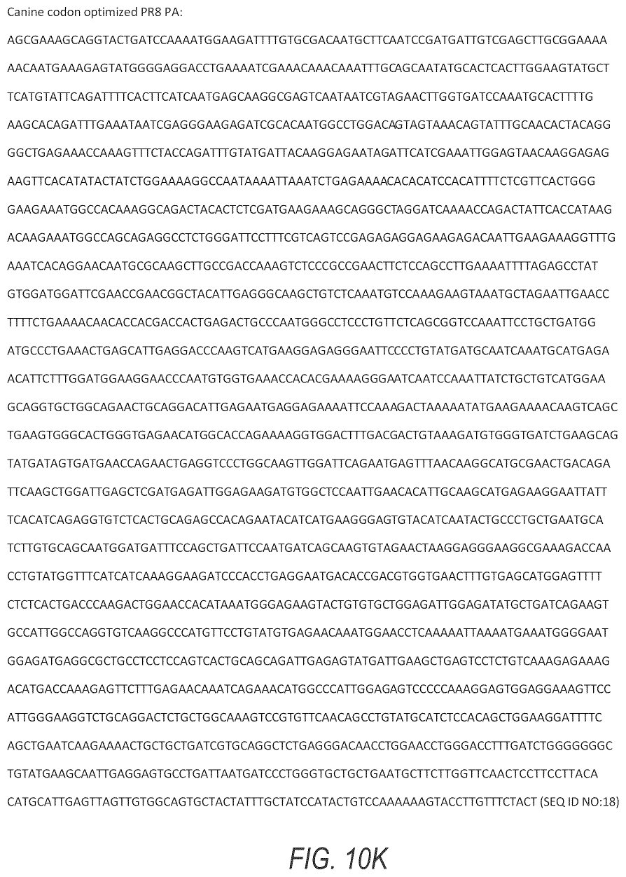

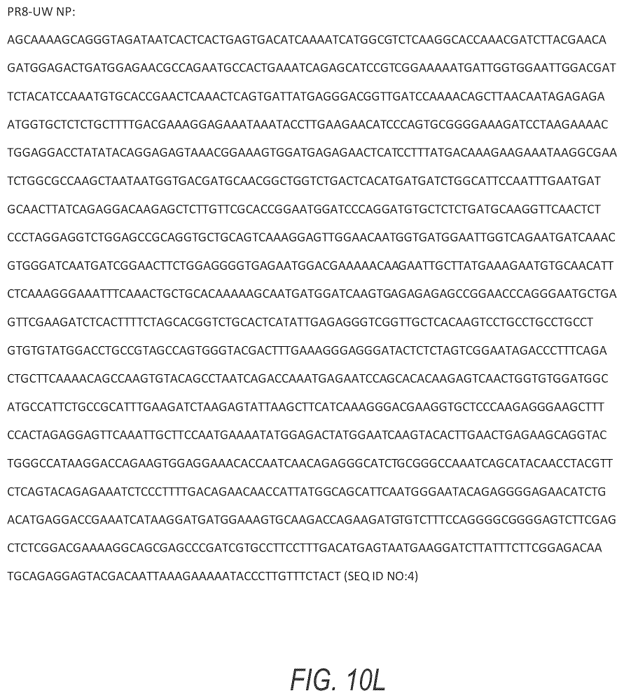

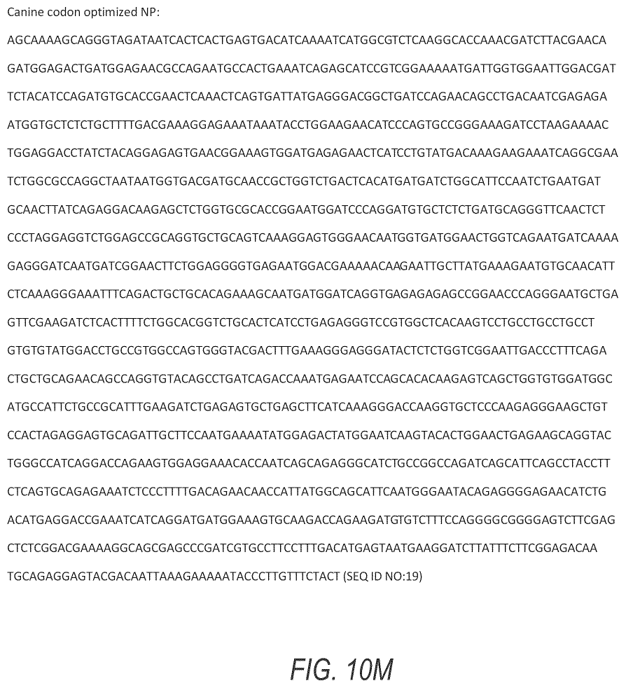

Optimization of rare codons in PB2 resulted in increased titers compared to wild type virus (UW-PR8) (see FIG. 10D). Other viral segments were codon optimized and titers of viruses with those segments or combinations of optimized segments were determined (FIG. 10E).

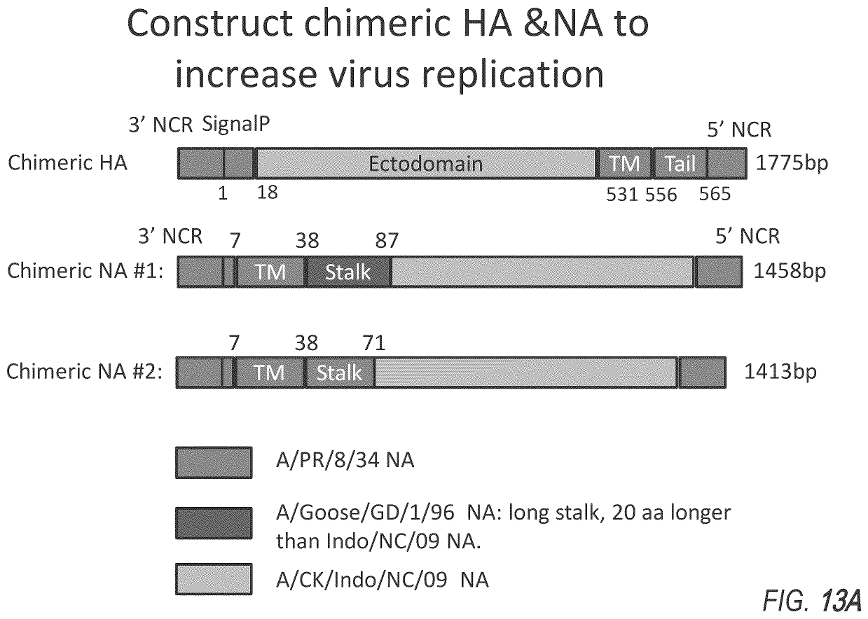

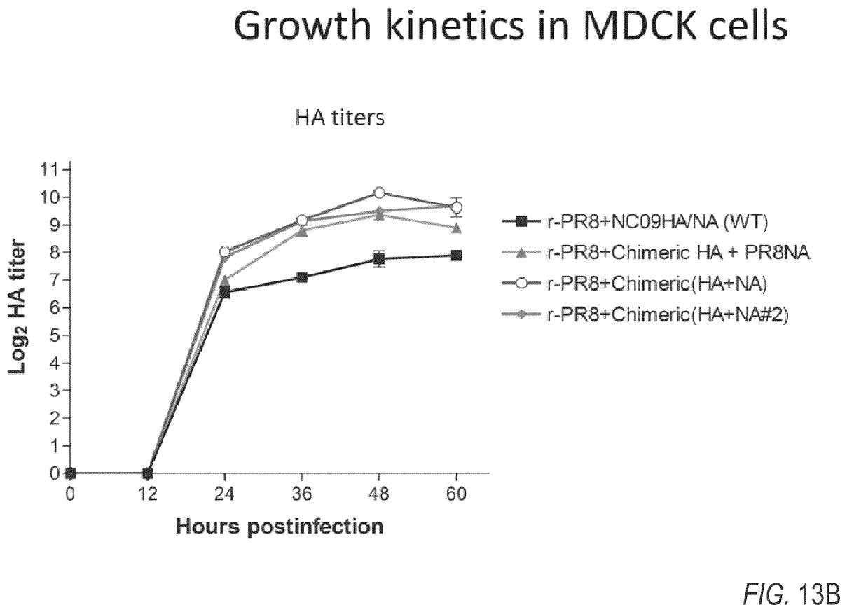

In another approach to increase virus titer in MDCK cells, chimeric HA and NA genes were prepared (FIG. 13A) and titers of viruses having those genes were determined (FIG. 13B).

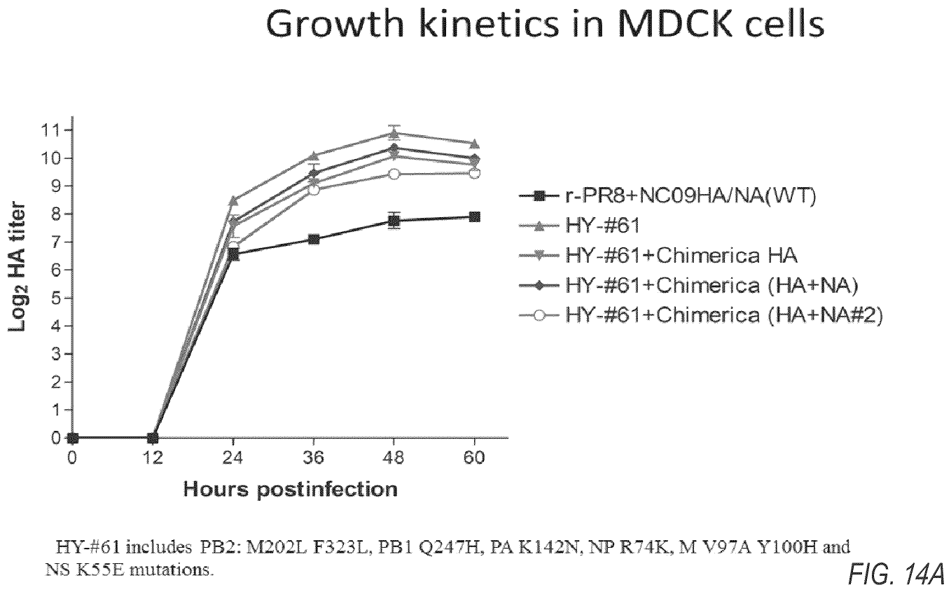

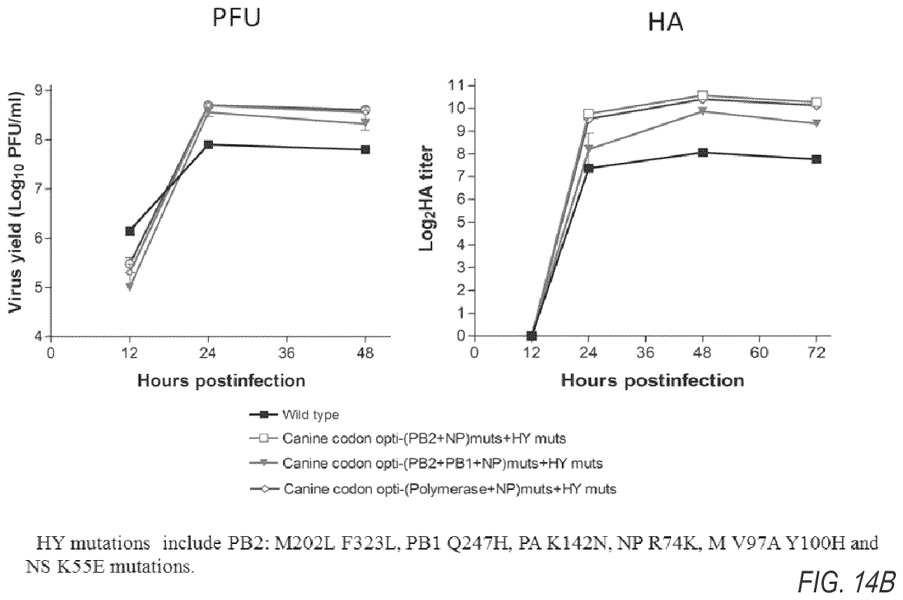

Viruses with combinations of the above-mentioned mutations (high growth backbone mutations, promoter mutations, chimeric HA and NA genes and canine codon optimization) were prepared and growth kinetics, PFU and HA titers of those viruses were determined (see FIG. 14). An exemplary set of backbone mutations are canine codon opti-PB2+C4U+M202L, F323L; PB1: C4U+Q247H; PA: C4U+K142N; NP: Canine codon opti-NP+R74K; M: V97A, Y100H; and NS: K55E.

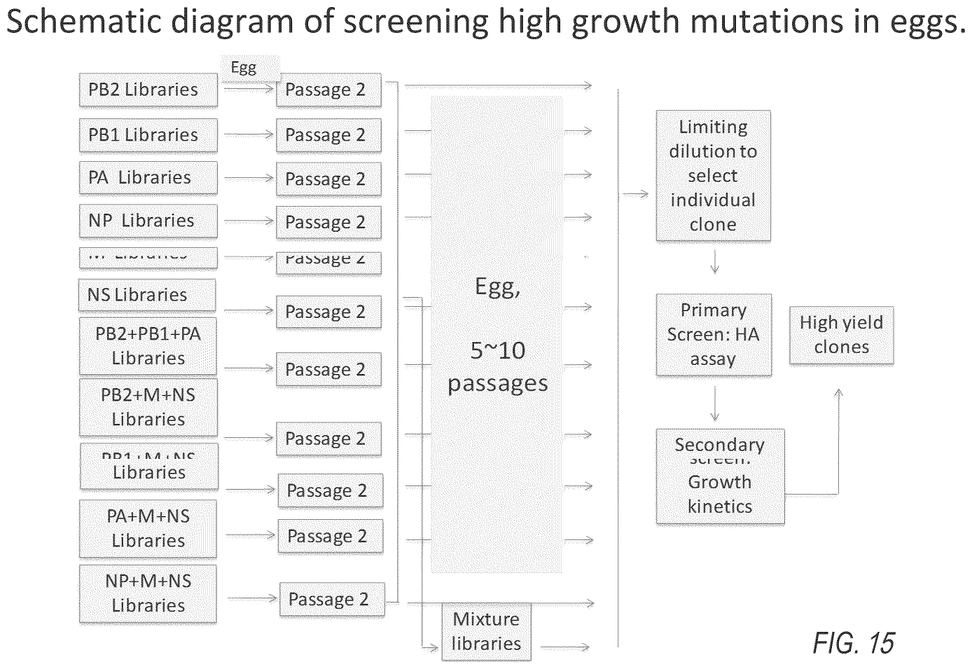

Any of the mutations described herein, or any combination thereof, may be combined with, for instance, seasonal H1N1 and H3N2, H3N2 Variant, PdmH1N1, H5N1, H7N9 or H9N2, or other clades or candidate vaccine strains. For example, HA and NA genes from A/California/04/2009(pdm H1N1) were combined with the six internal genes of UW-PR/8 to generate “6+2” recombinant viruses. Eleven virus libraries were generated and passaged 10 times in eggs. Three rounds of limiting dilution were performed to screen for high growth mutants (FIG. 15). In one embodiment, a variant with high growth properties in MDCK cells has a PB2 viral segment with a promoter mutation (C4U) and a mutation that results in I504V (relative to the parental virus); a PB1 viral segment with a promoter mutation (C4U) and a mutation that results in E112G; a PA viral segment with a promoter mutation (C4U) and a mutation that results in S225C; a NP viral segment with mutations that result in R74K and N417D; a M viral segment with mutations that result in V97A and Y100H; and a NS viral segment with a mutation that results in K55E, where optionally the sequence of one or more viral segments, e.g., the NP viral segment, is modified to include canine codon optimized codons. In one embodiment, a variant with high growth properties in MDCK cells has a canine codon optimized PB2 viral segment with a promoter mutation (C4U) and mutations that result in M202L and F323L; a PB1 viral segment with a promoter mutation (C4U) and a mutation that results in Q247H; a PA viral segment with a promoter mutation (C4U) and a mutation that results in K142N; a canine codon optimized NP viral segment with a mutation that results in R74K; a M viral segment with mutations that result in V97A Y100H; and a NS viral segment with a mutation that results in K55E.

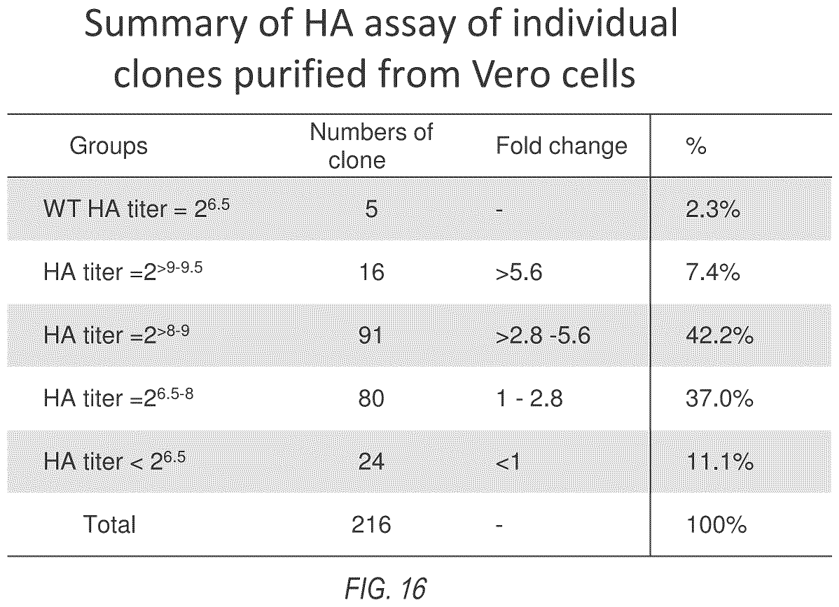

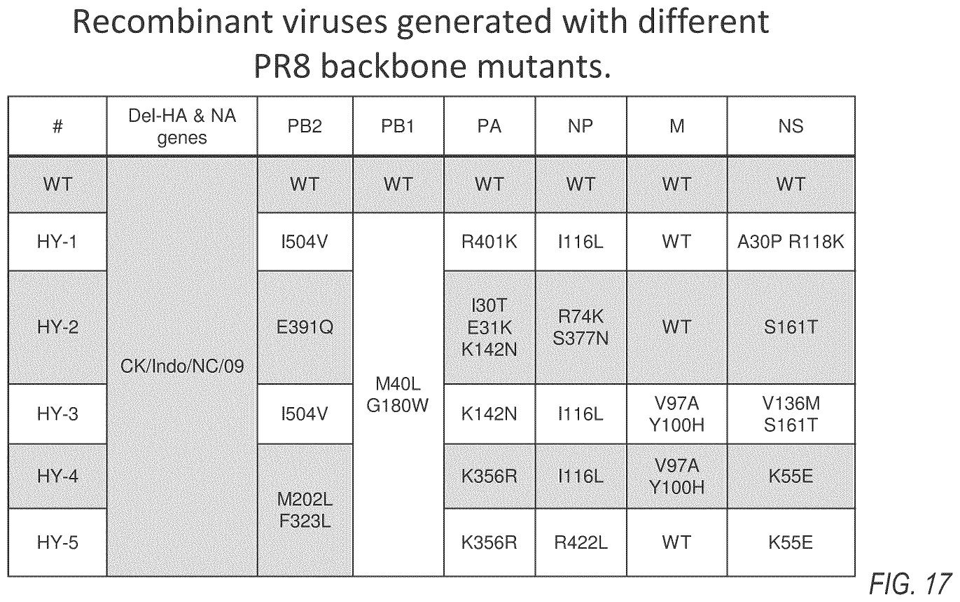

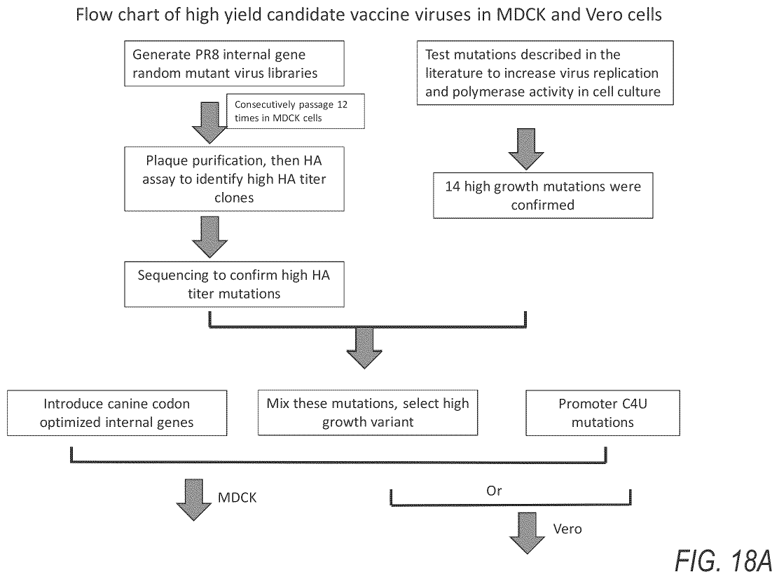

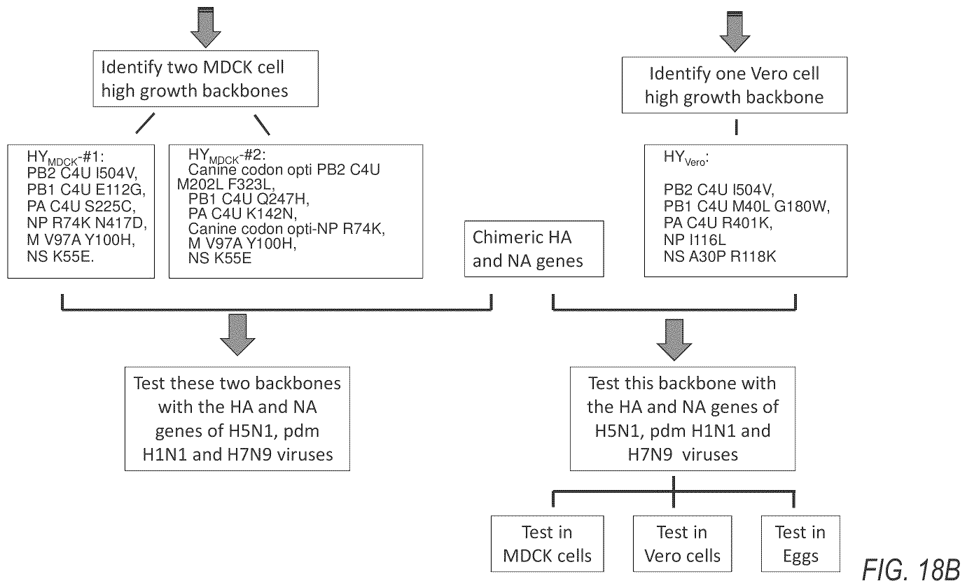

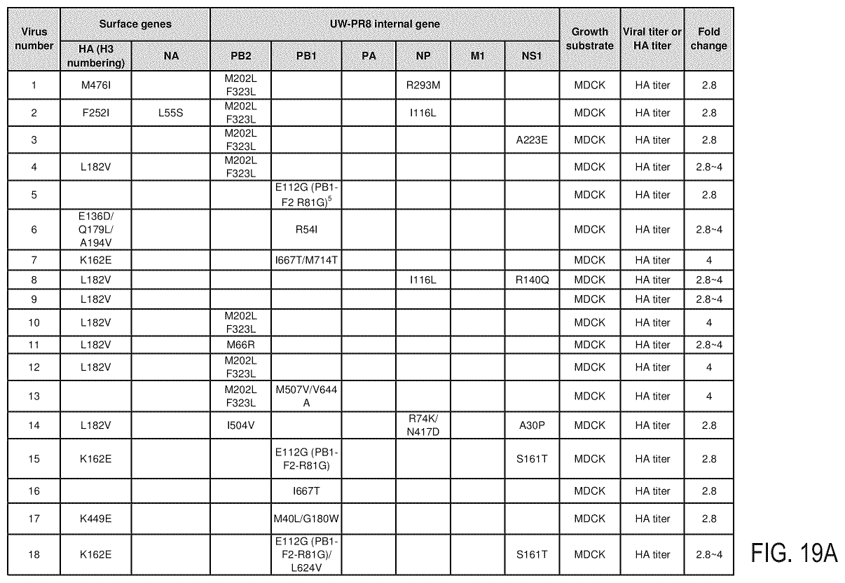

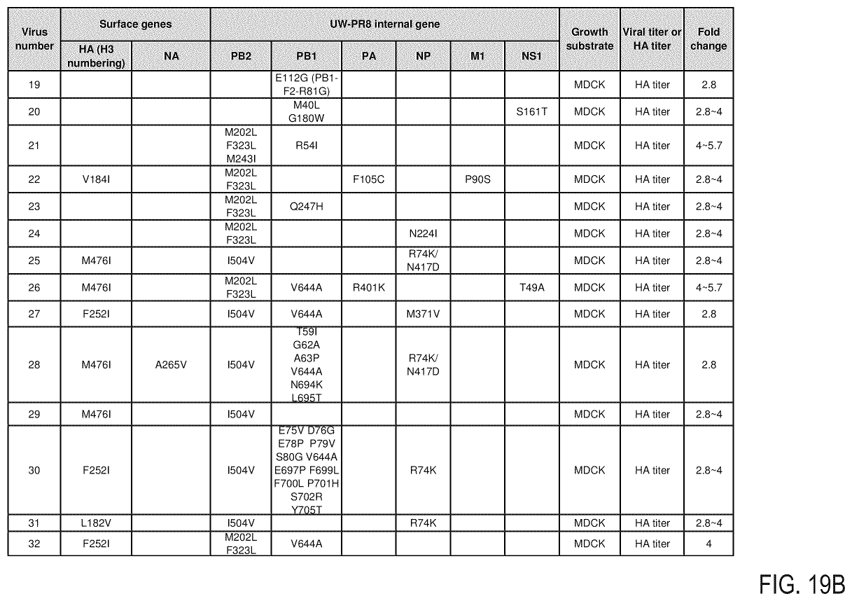

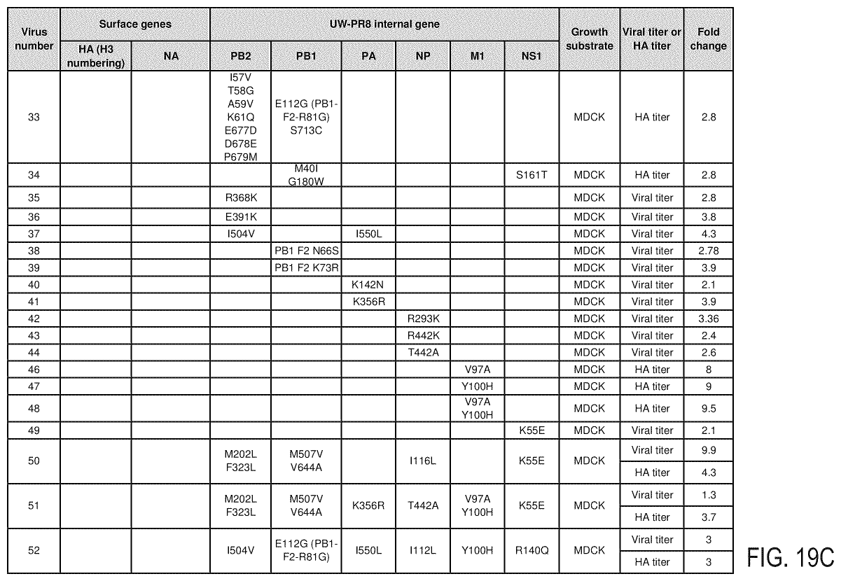

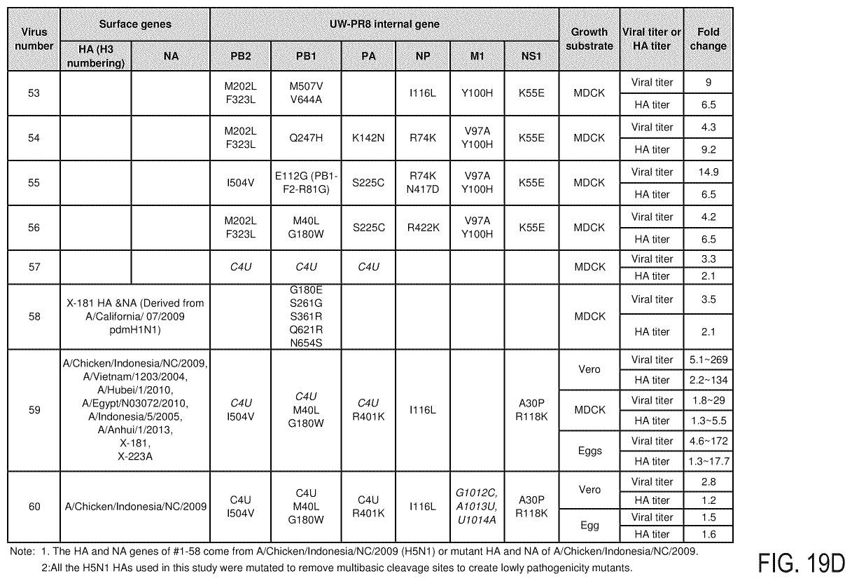

Similar experiments were conducted in Vero cells, e.g., after about 3 to 5 passages in Vero cells, using clones with high replicative properties in MDCK cells (see FIG. 16). FIG. 17 shows 5 viruses likely to have high replicative properties in Vero cells. In one embodiment, a PR8(UW) variant with high-growth properties in Vero cells has the following mutations that may be used in various combinations to increase the replicative ability of PR8(UW) viruses: PB2 segment: C4U (promoter mutation), I504V (amino acid change); PB1 segment: C4U (promoter mutation); M40L (amino acid change), G180W (amino acid change); PA segment: C4U (promoter mutation), R401K (amino acid change); NP segment: I116L (amino acid change); NS segment: A30P (amino acid change in NS1), or R118K (amino acid change in NS1).

In one embodiment, a PR8(UW) variant with high-growth properties has the following residues that may be used in various combinations with each other and/or other residues, e.g., those that enhance virus replication, to increase the replicative ability of reassortants having PR8(UW) based viral segment(s): a HA segment with one or more of 136D, 162E, 179L, 182V, 184I, 252I, 449E, and/or 476I: a NA segment with 55S and/or 265V; a NS segment with NS1 having 118K; F2 with 81G; a PB1 segment with 62A, 261G, 361R, 621R, and/or 654S, and/or viral segment promoters with the growth-enhancing nucleotides described herein, e.g., having one or more of the nucleotide changes G1012C, A1013U, or U1014A in the M viral segment.

### EXAMPLE 3

To assess the contribution of individual viral RNA (vRNA) segments to high-yield properties, a series of reassortant viruses was generated that possessed one or several vRNA segments of a high-yield PR8 (PR8-HY) variant in the background of the parental virus [UW-PR8_Indo/05(HA+NA)]. Vero cells were infected in triplicate with the indicated viruses at a MOI of 0.005 and incubated at 37° C. in the presence of trypsin. At the indicated time points, virus titers and HA titers were determined by performing plaque or HA assays, respectively. The results are shown in FIG. 20. These data indicated that several vRNA segments contribute to the properties of PR8-HY virus. In particular, the PB2+PB1+PA+NP vRNAs of PR8-HY virus conferred an appreciable increase in virus and HA titers, evidencing the enhanced replicative ability of this virus.

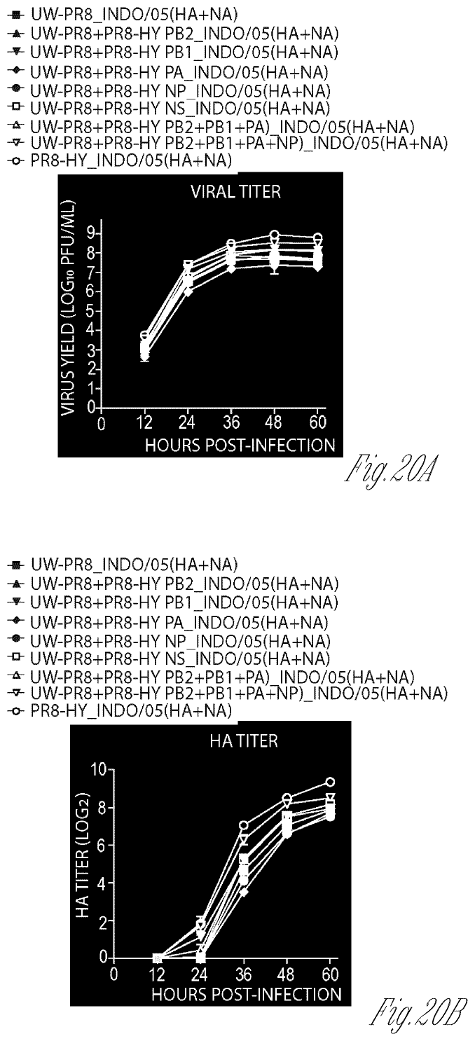

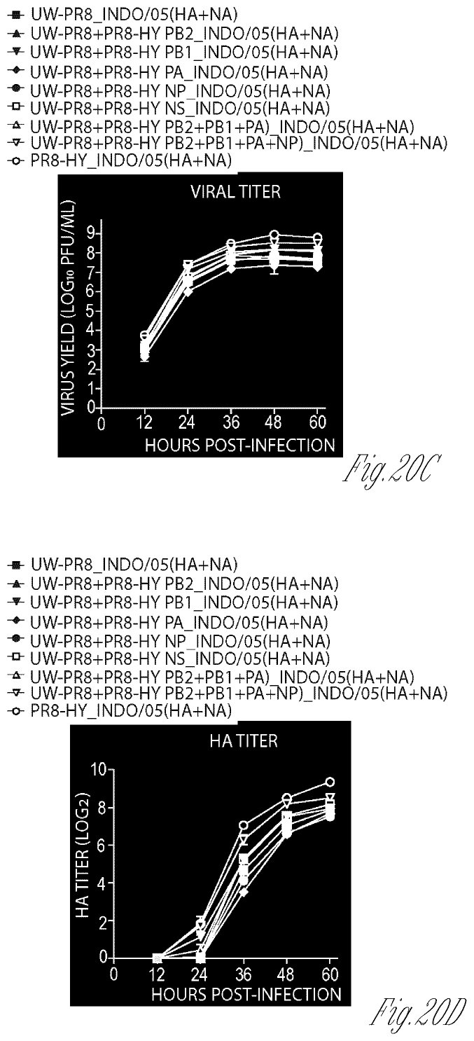

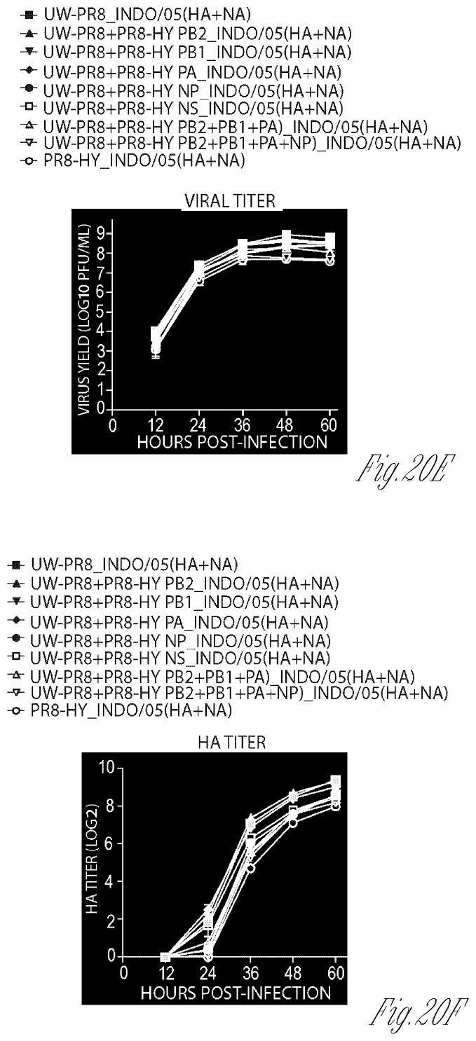

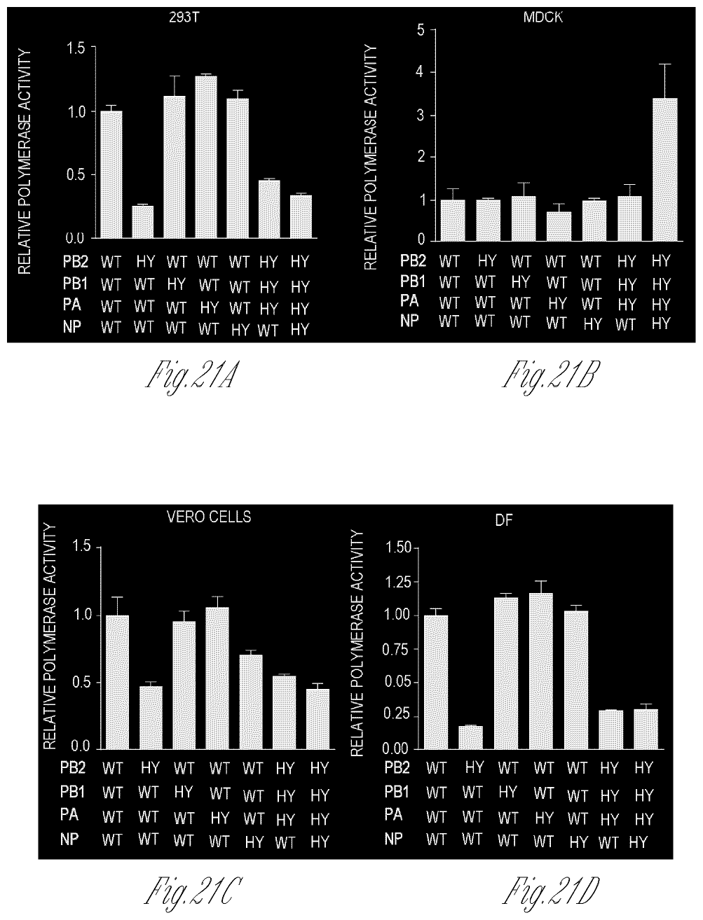

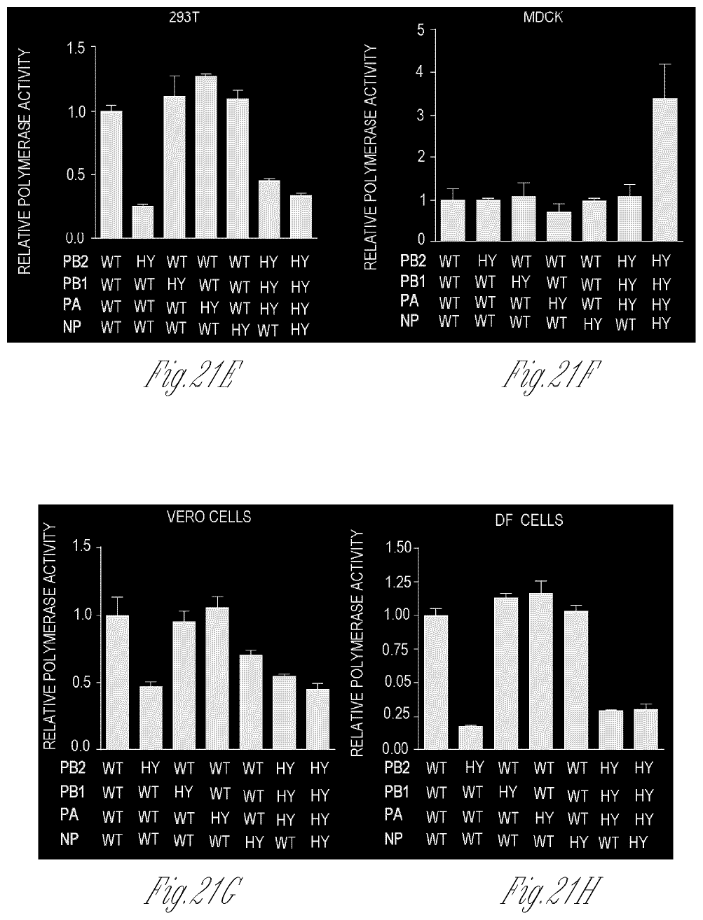

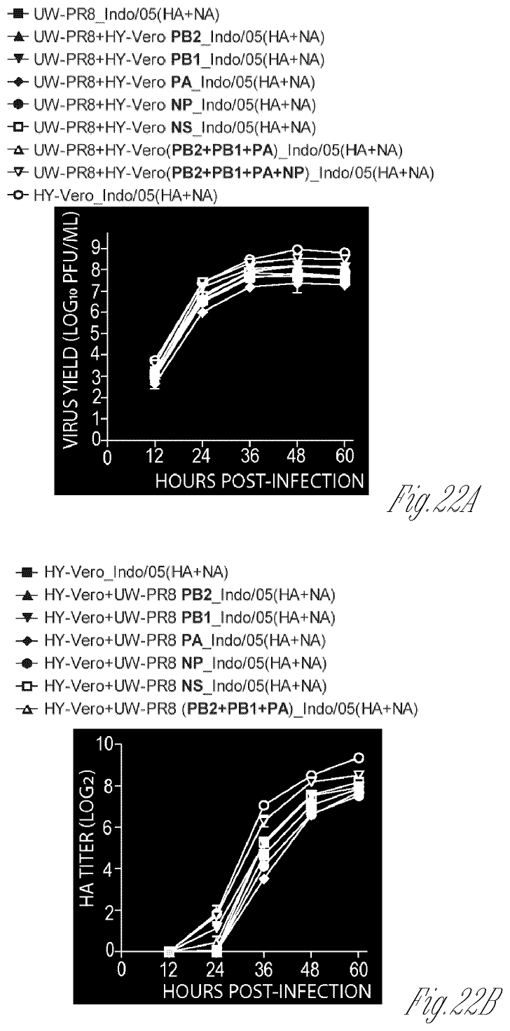

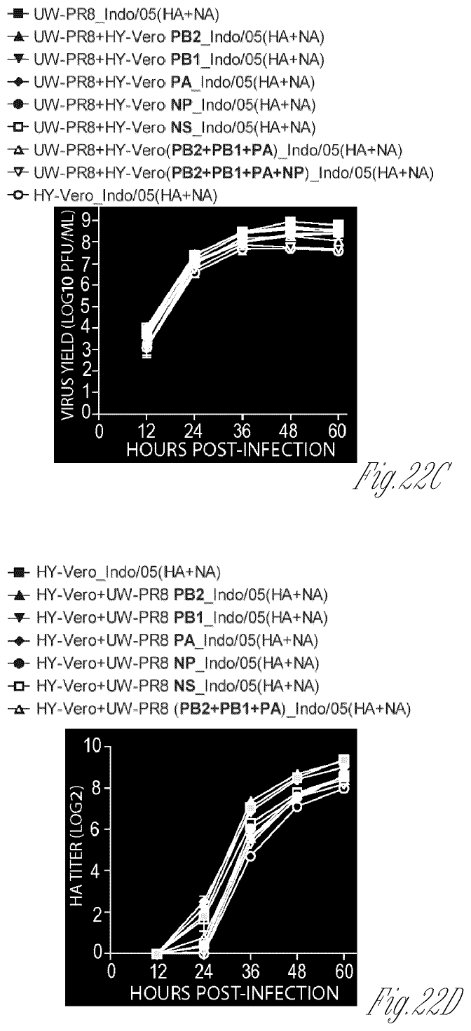

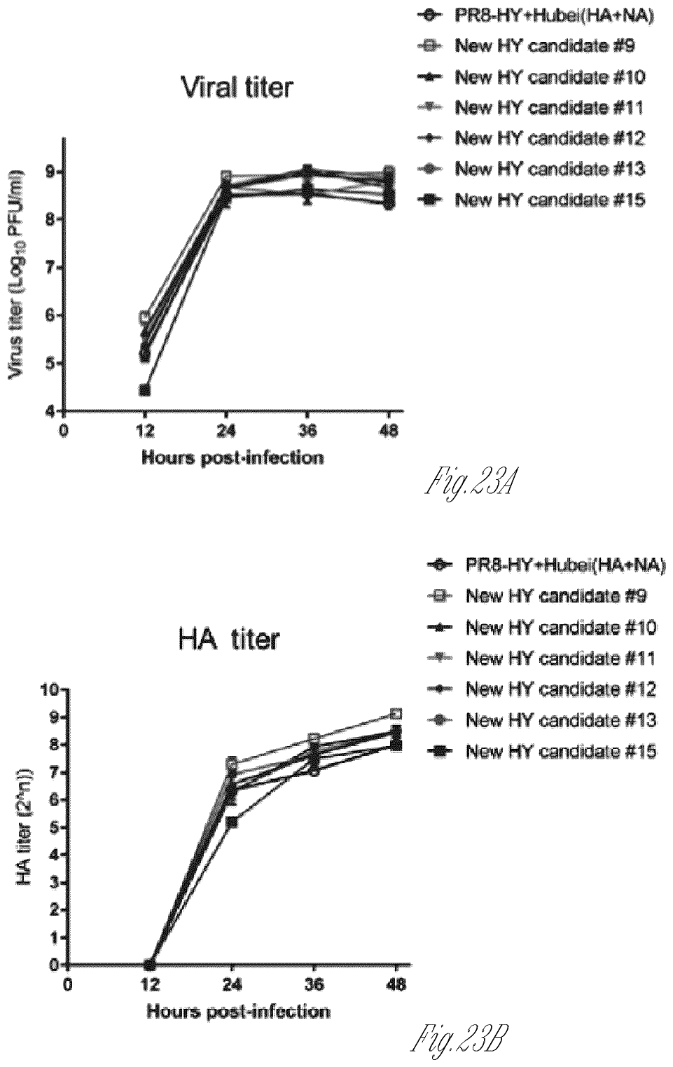

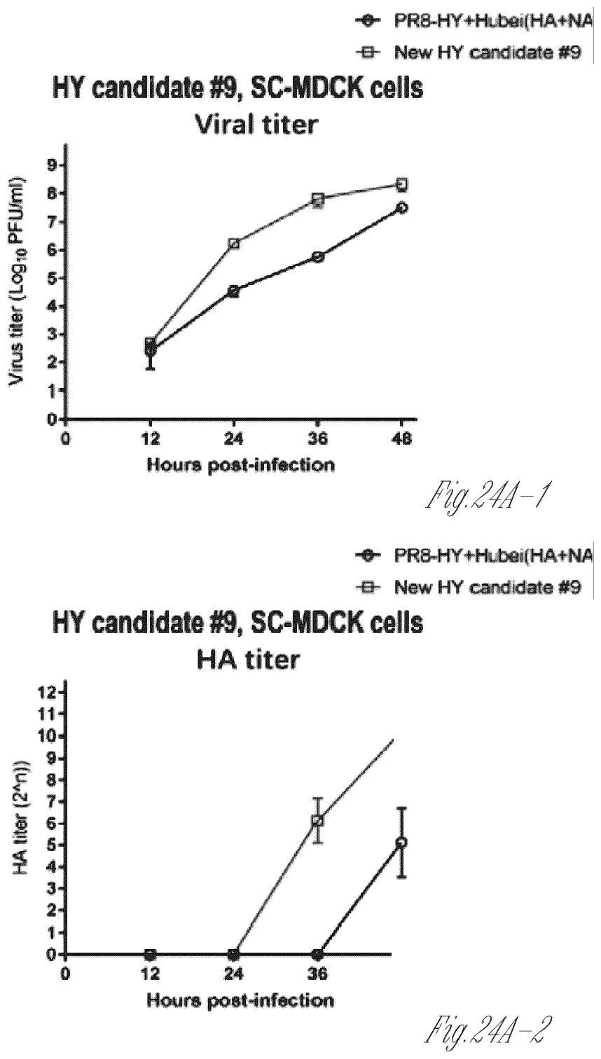

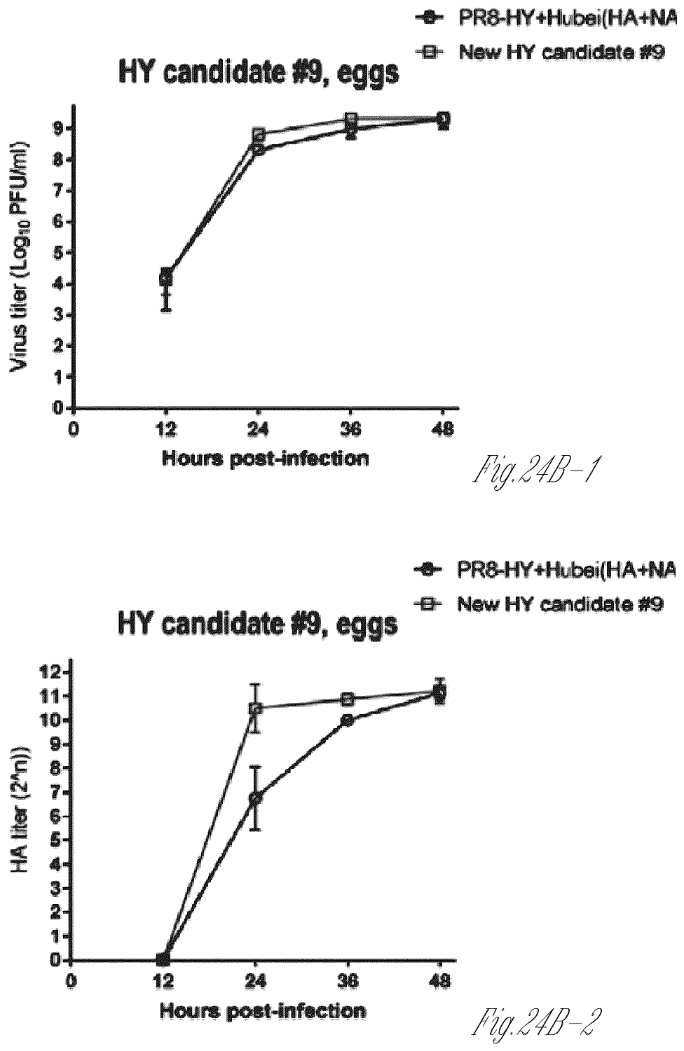

To further assess which component of the viral replication complex that provides for high-yield properties, wild-type or high-yield PB2, PB1, PA, and NP proteins were tested in various combinations in minireplicon assays in human 293T, canine MDCK, African green monkey Vero, and avian DF1 cells. The results are shown in FIG. 21. Interestingly, the PB2, PB1, PA, and NP proteins of PR8-HY virus attenuated the viral replicative ability in 293T, Vero, and DF1 cells; this effect was primarily conferred by the PB2 protein. In contrast, the combination of PB2+PB1+PA+NP proteins derived from PR8-HY virus conferred a substantial increase in replicated ability in canine MDCK cells, which were used for the selection of PR8-HY virus. The findings suggested host-dependent mechanisms underlying the high yield of PR8-HY virus. For example, the combination of PB1+PA+NP proteins, or a subset thereof, derived from PR8-HY may confer enhanced viral replicative ability in 293T, Vero, and DF1 cells.

Libraries were screened in serum-free (SF) medium, serum-containing (SC) medium, and eggs.

To identify a variant that replicates efficiently in all growth media tested, viruses were passaged under changing conditions:
SF to SC to Eggs, SF to Eggs to SC
SC to SF to Eggs, SC to Eggs to SF
Eggs to SF to SC, Eggs to SC to SF

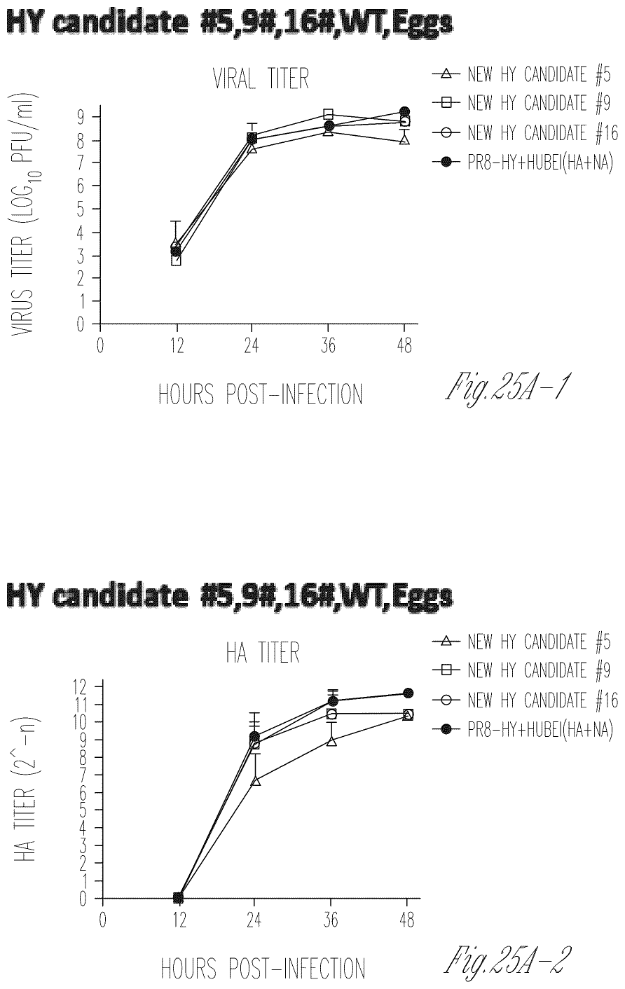

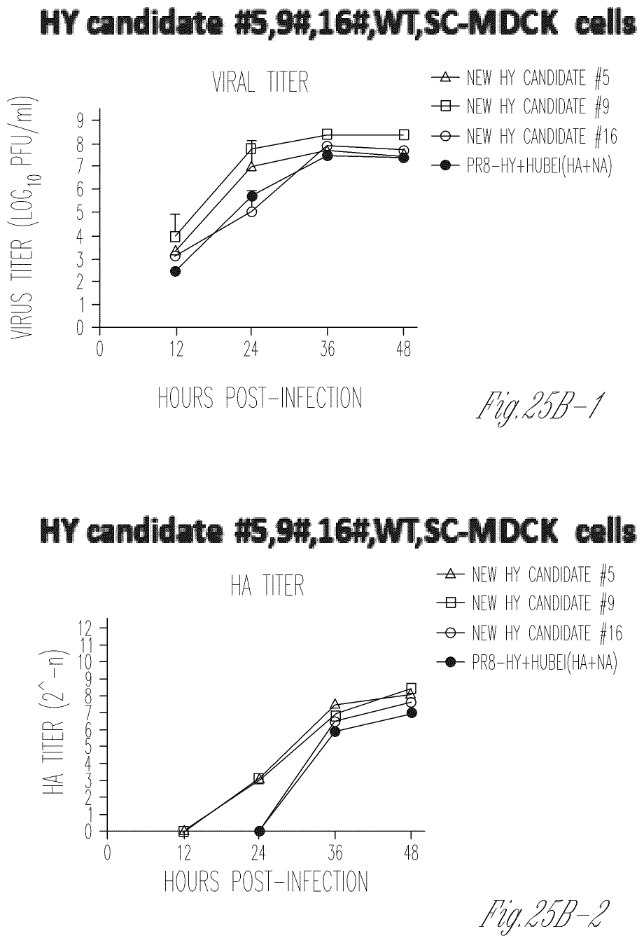

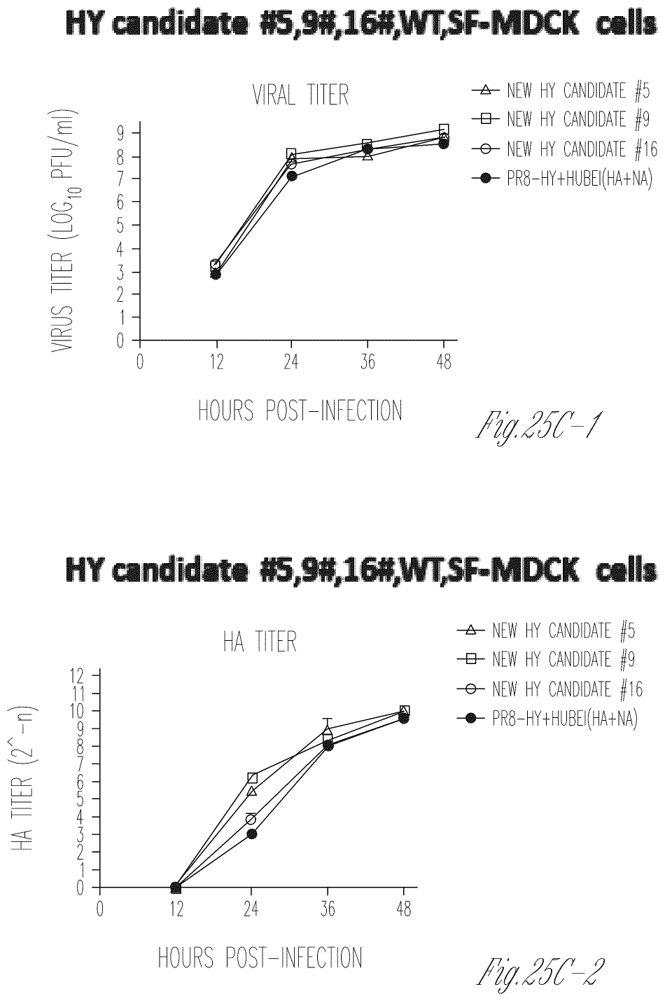

For example, other identified mutations that further improve the replication of influenza virus in cultured cells and/or embryonated chicken eggs are I711V in PB1 and M128L in the M1 protein, and those may be combined with any of the mutations disclosed herein, and in any combination (see FIGS. 23-25 and Tables 8-9). The viral titers for HY candidate #9 were 5.5-fold, 2.4-fold, 2.6-fold, 4.4-fold higher than those of the parental high yield backbone recombinant virus at 12 h, 24 h, 36 h and 48 h. HY candidate #9 contains the following mutations: PB2: C4U I504V; PB1: C4U M40L/G180W/I711V; PA: C4U R401K; NP: I116L; M1: M128L; NS1: A30P/R118K.

TABLE 8
Top 8 high HA titer clones selected from SF-MDCK PR8-HY + Hubei/1/10(H5N1) virus libraries

| | Virus stock titer | | | | | | | | | |
|---|---|---|---|---|---|---|---|---|---|---|
| | HA | | Mutations | | | | | | | |
| | titer | | | | | | | | HA (H3 | |
| Virus | (2\hat{ }n) | PFU/ml | PB2 | PB1 | PA | NP | M1 | NS1 | numbering) | NA |

| | | | | | | | | | |
|---|---|---|---|---|---|---|---|---|---|
| WT | — | — | C4U | C4U | C4U | I116L | | A30P/ | |
| | | | I504V | M40L/ | R401K | | | R118K | |
| | | | | G180W | | | | | |
| SF-14 | 10 | 1.44 × 10\hat{ }9 | | +I711V | | | +M128L | | K216N |
| SF-22 | 9.4 | 6.6 × 10\hat{ }8 | | +I711V | | | | | K216N |
| SF-37 | 9.6 | 8.7 × 10\hat{ }8 | | +I711V | | | | | K216N |
| SF-46 | 9.3 | 8.3 × 10\hat{ }8 | +M467I/ | | | | | | K22T/ |
| | | | K721R | | | | | | G326R |
| SF-42 | 9.5 | 8.7 × 10\hat{ }8 | | +I711V | +R256G | | | | K216N |
| SF-57 | 9.6 | 1 × 10\hat{ }9 | | | | | | | K222T |
| SF#15 | 10 | 1.24 × 10\hat{ }9 | +Y704H | | | | | | K222T |
| SF-E-85 | 9.6 | 9 × 10\hat{ }8 | | | +N350I/ | | | | K222T |
| | | | | | A369T | | | | |

TABLE 9
Top 12 HA titer clones selected from one round of passage of second generation mixed library (SC = serum containing; SF-serum-free.

| | | HA | | | | | | | | | |
|---|---|---|---|---|---|---|---|---|---|---|---|
| | | liter | | | | | | | | | |
| Medium | Plaque # | (2\hat{ }n) | HA & NA | PB2 | PB1 | PA | NP | M1 | M2 | NS1 | NS2 |
| | WT | | Detoxified- | C4UI504V | C4U | C4U | I116L | | | A30P/ | |
| | | | A/Hubei/1/ | | M40L/ | R401K | | | | R118K | |
| | | | 2010(HA + | | G180W | | | | | | |
| SC | 11 | 7.5 | NA) | C4UI504V + | C4U | C4U | I116L + | S126G + | W15G | A30P/ | 176M |
| ↓ | | | | A221V | M40L/ | R401K | K103R + | 244 (mixture) | | R118K + | |
| Egg | | | | | G180W | | V194I | M/R | | D101V | |
| ↓ | 14 | 7.5 | | C4UI504V + | C4U | C4U | I116L + | M128L + | W15G | A30P/ | E820 |
| SF | | | | A221V | M40L/ | R401K | K103R + | 244 (mixture) | | R118K | |
| | | | | | G180W + | | V194I | M/R | | | |
| | | | | | I711V | | | | | | |
| | 23 | 8.0 | | C4UI504V + | C4U | C4U | I116L + | F62L + | W15G | A30P/ | |
| | | | | R62S + | M40L/ | R401K | K103R + | 244 (mixture) | | R118K | |
| | | | | A370T | G180W + | | V194I | M/R | | | |
| | | | | | I711V | | | | | | |
| | 27 | 7.5 | | C4UI504V + | C4U | C4U | I116L | S126G | W15G | A30P/ | |
| | | | | R62S | M40L/ | R401K | | | | R118K | |
| | | | | | G180W + | | | | | | |
| | | | | | E178K + | | | | | | |
| | | | | | M290I | | | | | | |
| | 33 | 7.5 | | C4UI504V | C4U | C4U | I116L | S126G + | W15G | A30P/ | I76M |
| | | | | | M40L/ | R401K + | | 244 (mixture) | | R118K + | |
| | | | | | G180W + | K142E | | M/R | | D101V | |
| | | | | | I711V | | | | | | |
| SC | 1 | 7.5 | | C4UI504V + | C4U | C4U | I116L | S126G + | W15G | A30P/ | N92D |
| ↓ | | | | R62S + | M40L/ | R401K + | | 244 (mixture) | | R118K | |
| SF | | | | A370T | G180W + | R256G | | M/R | | | |
| ↓ | | | | | M92I | | | | | | |
| Egg | 2 | 7.5 | | C4UI504V + | C4U | C4U | I116L | M128L + | | A30P/ | I76M |
| | | | | A221V | M40L/ | R401K + | | 244 (mixture) | | R118K + | |
| | | | | | G180W + | R256G | | M/R | | D101V | |
| | | | | | M92I | | | | | | |
| | 4 | 7.5 | | C4UI504V + | C4U | C4U | I116L | 244(mixture) | W15G | A30P/ | |
| | | | | R62S | M40L/ | R401K + | | M/R | | R118K | |
| | | | | | G180W + | R256G | | | | | |
| | | | | | M325V | | | | | | |
| | 5 | 8.0 | | C4UI504V + | C4U | C4U | I116L | S126G + | W15G | A30P/ | E82D |
| | | | | A221V | M40L/ | R401K + | | 244 (mixture) | | R118K | |
| | | | | | G180W + | K142E | | M/R | | | |
| | | | | | M92I | | | | | | |
| | 13 | 8.0 | | C4UI504V | C4U | C4U | I116L + | M128L + | W15G | A30P/ | N92D |
| | | | | | M40L/ | R401K + | K103R + | 244 (mixture) | | R118K | |
| | | | | | G180W + | K142E | V194I | M/R | | | |
| | | | | | M92I | | | | | | |
| | 24 | 8.0 | | C4UI504V+ | C4U | C4U | I116L + | M128L + | W15G | A30P/ | E82D |
| | | | | R62S + | M40L/ | R401K + | K103R + | 244 (mixture) | | R118K | |
| | | | | A370T | G180W | K142E | V194I | M/R | | | |
| | 25 | 7.5 | | C4UI504V | C4U | C4U | I116L + | S126G + | W15G | A30P/ | N92D |
| | | | | | M40L/ | R401K | K103R + | 244 (mixture) | | R118K | |
| | | | | | G180W + | | V194I | M/R | | | |
| | | | | | M92I + | | | | | | |
| | | | | | N105H | | | | | | |

### References

Avery's Drug Treatment: Principles and Practice of Clinical Pharmacology and Therapeutics, 3rd edition, ADIS Press, Ltd., Williams and Wilkins, Baltimore, MD (1987).
Aymard-Henry et al., Virology: A Practical Approach, Oxford IRL Press, Oxford, 119-150 (1985).
Bachmeyer, Intervirology, 5:260 (1975).
Berkow et al., eds., The Merck Manual, 16th edition, Merck & Co., Rahway, NJ (1992).
Hatta et al., Science, 293:1840 (2001).
Horimoto et al., J. Virol., 68:3120 (1994).
Horimoto et al., Vaccine, 24:3669 (2006).
Keitel et al., in Textbook of Influenza, eds. Nickolson, K. G., Webster, R. G., and Hay, A. (Blackwell, Oxford), pp. 373-390 (1998).
Laver & Webster, Virology, 69:511 (1976).
Neumann et al., Adv. Virus Res., 53:265 (1999).
Neumann et al., J. Gen. Virol., 83:2635 (2002).
Neumann et al., J. Virol., 71:9690 (1997).
Neumann et al., Proc. Natl. Acad. Sci. USA, 96:9345 (1999).
Neumann et al., Virology, 287:243 (2001).
Osol (ed.), Remington's Pharmaceutical Sciences, Mack Publishing Co., Easton, PA 1324-1341 (1980).
Sugawara et al., Biologicals, 30:303 (2002).
Webby & Webster et al., Science, 302:1519 (2003).
Wood & Robertson, Nat. Rev. Microbiol., 2:842 (2004).
World Health Organization TSR No. 673 (1982).
World Health Organization. Confirmed human cases of avian influenza A (H5N1). http://www.who.int/csr/disease/avian_influenza/country/en/index.html

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A plurality of influenza virus vectors for preparing a reassortant, comprising
a vector for vRNA production comprising a promoter operably linked to an influenza virus PA DNA linked to a transcription termination sequence, a vector for vRNA production comprising a promoter operably linked to an influenza virus PB1 DNA linked to a transcription termination sequence, a vector for vRNA production comprising a promoter operably linked to an influenza virus PB2 DNA linked to a transcription termination sequence, a vector for vRNA production comprising a promoter operably linked to an influenza virus HA DNA linked to a transcription termination sequence, a vector for vRNA production comprising a promoter operably linked to an influenza virus NP DNA linked to a transcription termination sequence, a vector for vRNA production comprising a promoter operably linked to an influenza virus NA DNA linked to a transcription termination sequence, a vector for vRNA production comprising a promoter operably linked to an influenza virus M DNA linked to a transcription termination sequence, and a vector for vRNA production comprising a promoter operably linked to an influenza virus NS cDNA linked to a transcription termination sequence, wherein the PB1, PB2, PA, NP, NS, and M DNAs in the vectors for vRNA production are from one or more influenza vaccine virus isolates, wherein the NA DNA in the vector for vRNA production of NA has sequences for a heterologous NA, and wherein the HA DNA in the vector for vRNA production of HA has sequences for a heterologous HA, wherein the PB1 DNA encodes a PB1 with a residue other than isoleucine at position 711 or the M DNA encodes a M1 with a residue other than methionine at position 128; and
a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2.

2. The vectors of claim 1, wherein the PB1 viral segment encodes a PB1 with a residue other than isoleucine at position 711 and the M viral segment encodes a M1 with a residue other than methionine at position 128; or wherein the PA viral segment encodes a PA with a residue other than phenylalanine at position 105, a residue other than lysine at position 142, a residue other than serine at position 149, a residue other than serine at position 225, a residue other than lysine at position 356, a residue other than threonine at position 357, a residue other than arginine at residue 401, or a residue other than isoleucine at position 550, or any combination thereof; or wherein the PB1 viral segment further encodes a PB1 with a residue other than methionine at position 40, a residue other than glutamic acid at position 112, a residue other than glycine at position 180, or a residue other than glutamine at residue 247, or
any combination thereof; or wherein the PB2 viral segment encodes a PB2 a residue other than methionine at position 202, a residue other than phenylalanine at position 323, or a residue other than isoleucine at position 504, or any combination thereof or wherein the NP viral segment encodes a NP with a residue other than arginine at position 74, a residue other than isoleucine at position 116, or a residue other than asparagine at position 417, or any combination thereof; or wherein the NS viral segment encodes a NS1 with a residue other than alanine at position 30, a residue other than lysine at position 55, or a residue other than arginine at position 118, or any combination thereof.

3. The vectors of claim 1, wherein the virus has a V, A, L, G, S, T, C, or M at position 711 in PB1, has a A, L, G, V, S, T, I, or C at position 40 in PB1, has a A, L, W, Y, F, V, S, T, I, or C at position 180 in PB1; has a L, V, G, A, S, C or T at position 116 in NP; has a P, W, F, L, I, V, G, S, C or T at position 30 in NS1, has a K, H, D, E, Q, or N at position 118 in NS1; has a L, I, V, G, A, S, C or T at position 128 in M1; has a K, H, D, E, N or Q at position 401 in PA; has a V, A, L, G, S, T, C, or M at position 504 in PB2; or any combination thereof; or wherein the virus has a V, A, L, G, S, T, C, or M at position 711 in PB1, has a H, R, K, D, E, D or E at position 247 in PB1; has a K, H, D, E, Q or N at position 74 in NP; has E, D, Q, or N at position 55 in NS1; has a L, I, V, G, A, S, C or T at position 128 in M1; has a C, L, I, V, G, A, or T at position 142 in PA; has a A, L, G, S, T, I, or C at position 202 in PB2, has a A, L, G, S, T, I, or C at position 323 in PB2; or any combination thereof.

4. The vectors of claim 1, wherein the virus has a V, A, L, G, S, T, C, or M at position 711 in PB1, has a A, L, G, V, S, T, I, or C at position 112 in PB1; has a K, H, D, E, Q or N at position 74 in NP, has a D, E, Q, K, or H at position 417 in NP; has E, D, Q, or N at position 55; has a L, I, V, G, A, S, C or T at position 128 in M1; has a C, L, I, V, G, A, or T at position 225 in PA; has an V, A, L, G, S, T, C, or M at position 504 in PB2; or any combination thereof; or wherein the virus has one or more of 142N, 225C, 356R, or 550L in PA; has one or more of 112G, 247H, 507V, or 644A in PB1; has one or more of 202L, 323L or 504V in PB2; has one or more of 74K, 112L, 116L, 417D, or 442A in NP; 97A and/or 100H in M1; and/or 55E and/or 140Q in NS1, or any combination thereof.

5. The vectors of claim 1, wherein the virus has at least one of 202L and/or 323L in PB2, 247H in PB1 or 74K in NP and optionally at least one of 142N in PA1, 55K in NS1 or 97A and/or 100H in M1; or wherein the virus has at least one of 202L and/or 323L in PB2, 247H in PB1 or 74K in NP and at least one of 142N in PA1, 55K in NS1 or 97A and/or 100H in M1; or wherein the virus has 202L and/or 323L in PB2; or wherein the virus has 247H in PB1; or wherein the virus has 55E in NS1; or wherein the virus has 142C in PA; or wherein the virus has 74K in NP; or wherein the virus has 504V in PB2; or wherein the virus has 74K, 116L or 417D in NP; or wherein the virus has 30P, 55E or 118K in NS1; or wherein the virus has 225C or 401K in PA; or wherein the virus has a V, A, L, G, S, T, C, or M at position 711 in PB1.

6. The vectors of claim 1, wherein the virus has a L, I, V, G, A, S, C or T at position 128 in M1.

7. The vectors of claim 1, wherein the PB1, PB2, PA, NP, NS, and M DNAs in the vectors for vRNA production have a sequence corresponding to one that encodes a polypeptide having at least 95% amino acid sequence identity to a corresponding polypeptide encoded by SEQ ID NOS:1-6 or 10-15.

8. The vectors of claim 1, wherein the promoter for vRNA vectors is a RNA polymerase I promoter, a RNA polymerase II promoter, a RNA polymerase III promoter, a T3 promoter or a T7 promoter.

9. The vectors of claims 1, wherein the HA is H1, H2, H3, H5, H6, H7, H9 or H10.
